(19) United States
(12) Patent Application Publication
(10) Pub. No.: US 2023/0108800 A1
(43) Pub. Date: Apr. 6, 2023

(54) INFUSION PUMP WITH MULTIPLE RESERVOIRS

(71) Applicant: Quasuras, Inc., Escondido, CA (US)

(72) Inventors: David NEESE, Escondido, CA (US); Marc GOLDMAN, San Diego, CA (US); Paul M. DIPERNA, Escondido, CA (US)

(21) Appl. No.: 17/955,174

(22) Filed: Sep. 28, 2022

Related U.S. Application Data
(60) Provisional application No. 63/249,802, filed on Sep. 29, 2021.

(51) Int. Cl. A61M 5/142; A61M 5/145

(52) U.S. Cl. CPC A61M 5/14248; A61M 5/1452; A61M 2005/14533; A61M 2205/128; A61M 2205/3327; A61M 2205/3331; A61M 2205/8206

(57) ABSTRACT

Rotary microfluidic medical pump and pumping method embodiments are discussed herein that may be used for controlled delivery of small and precise volumes of therapeutic or non-therapeutic fluids in a variety of environmental conditions. Certain medical pump and pumping method embodiments discussed herein may also include pump systems having a plurality of reservoirs and which are configured for pumping a plurality of fluids such as medicaments via a common pump mechanism and outlet tubing.

### RELATED APPLICATIONS

This application claims priority from U.S. Provisional Pat. Application Serial No. 63/249,802 filed Sep. 29, 2021, by David NEESE et al., and titled “INFUSION PUMP WITH MULTIPLE RESERVOIRS”, which is incorporated by reference herein in its entirety.

### BACKGROUND

The delivery of therapeutic and non-therapeutic medical fluids is commonly performed intravenously (IV) or subcutaneously using an infusion catheter or cannula and a syringe type of pump. The mechanism of such syringe type pumps typically advances a plunger to compress a fluid within a corresponding syringe housing in a controlled fashion to advance the fluid and provide accurate dosing. For ambulatory pumps this mechanism may be scaled down but generally the small, powerful, and accurate motor, gear train and encoder that is required for such syringe pumps is expensive. Syringe pumps typically also rely on a motor driven lead screw and attachment to a compressing plunger to not only control the delivery of fluids but also to prevent the unexpected delivery of fluids to a patient while in use by the patient.

In some circumstances, the accuracy of syringe type pumps as well as other common medical pumps, including peristaltic type pumps, may be compromised by changes in environmental conditions including variation in ambient temperature, changes in ambient pressure as well as other factors. What have been needed are improved pumping mechanisms and methods that are reasonably priced and that can reliably deliver small quantities of medical fluids in an accurate and consistent manner without susceptibility to environmental variations.

### SUMMARY

Some embodiments of a medical pump system may include a reservoir cartridge assembly having a first fluid source, a second fluid source, and a reservoir selector assembly having a first selector valve assembly disposed in fluid communication with the first fluid source and second selector valve assembly disposed in fluid communication with the second fluid source. The reservoir cartridge assembly may also include a pump chamber assembly having a pump chamber with an interior volume, an inlet port in fluid communication with the reservoir selector assembly and an outlet port in fluid communication with the interior volume and with an outlet conduit. An actuator assembly of the medical pump system may be configured to be operatively and releasably coupled to the reservoir cartridge assembly. The actuator assembly may include a pump chamber actuator, a motor operatively coupled to the pump chamber actuator and a controller operatively coupled to the motor.

Some embodiments of a method of delivering multiple fluids to a patient from a medical pump system may include actuating a pump chamber assembly with a cam shaft of a cam assembly by rotating the cam shaft in a pumping direction and pumping a first fluid from a first fluid source through a first valve assembly of a reservoir selector assembly and a pump chamber assembly of the medical pump system into an outlet conduit and to the patient. Thereafter, the method may include reversing the rotation of the cam shaft of the cam assembly from the pumping direction to a valve selection direction and ceasing pumping of the first fluid from the first fluid source. A cam segment of the cam assembly may then be rotated in the valve selection direction with a one way coupling disposed between and operatively coupling the cam shaft to the cam segment to close the first valve assembly and open a second valve assembly. Then the method embodiment may include reversing the rotation of the cam shaft from the valve selection direction to the pumping direction and pumping a second fluid from the second fluid source through the second valve assembly and pump chamber assembly of the medical pump system into the outlet conduit and to the patient.

Some embodiments of a method of delivering multiple fluids to a patient from a medical pump system may include actuating a pump chamber assembly with a cam shaft of a cam assembly by rotating the cam shaft in a pumping direction and pumping a first fluid from a first fluid source through a first valve assembly of a reservoir selector assembly and a pump chamber assembly of the medical pump system into an outlet conduit and to the patient while a second valve assembly and third valve assembly of the reservoir selector assembly are closed. Thereafter, the method may include reversing the rotation of the cam shaft of the cam assembly from the pumping direction to a valve selection direction, ceasing pumping of the first fluid from the first fluid source and actuating the reservoir selector assembly by rotating a cam segment of the cam assembly in a valve selection direction with a one way coupling disposed between and operatively coupling the cam shaft to the cam segment to close the first valve assembly, close the third valve assembly and open the second valve assembly. The method embodiment may also then include reversing the rotation of the cam shaft from the valve selection direction to the pumping direction and pumping a second fluid from a second fluid source through the second valve assembly and pump chamber assembly of the medical pump system into the outlet conduit and to the patient while the first valve assembly and the third valve assembly are closed. After pumping the second fluid the rotation of the cam shaft of the cam assembly may again be reversed from the pumping direction to the valve selection direction, ceasing pumping of the second fluid from the second fluid source and rotating the cam segment of the cam assembly in the valve selection direction, and closing the first valve assembly, closing the second valve assembly and opening the third valve assembly. Finally, the rotation of the cam shaft may again be reversed from the valve selection direction to the pumping direction and pumping a third fluid from a third fluid source through the third valve assembly and pump chamber assembly of the medical pump system into the outlet conduit and to the patient while the first valve assembly and second valve assembly are closed. For some such method embodiments, pumping the first fluid may include pumping a first therapeutic liquid through the pump chamber and outlet conduit, pumping the second fluid may include pumping a bolus of filtered air through the pump chamber and outlet conduit after pumping the first therapeutic liquid, and pumping the third fluid may include pumping a second therapeutic liquid through the pump chamber and outlet conduit after pumping the bolus of filtered air therethrough. In some cases, pumping the bolus of filtered air may include forming a meniscus at a boundary between the bolus of filtered air and the first therapeutic liquid and forming a meniscus at the boundary between the bolus of filtered air and the second therapeutic liquid.

Some embodiments of a method of delivering multiple fluids to a patient from a medical pump system may include pumping a first liquid from a first fluid source of the medical pump system into an outlet conduit and to the patient. Thereafter, ceasing pumping of the first liquid and pumping a gas from a second fluid source into the outlet conduit and to the patient then ceasing pumping of the gas from the second fluid source and pumping a third liquid from a third fluid source into the outlet conduit and to the patient.

Some embodiments of a medical pump system may include a reservoir cartridge assembly having a first fluid reservoir, a second fluid reservoir, and a reservoir selector assembly in fluid communication with the first fluid reservoir and the second fluid reservoir. In some instances, the reservoir cartridge assembly may include the first fluid reservoir, the second fluid reservoir, an optional third fluid reservoir or air system and a reservoir selector assembly in fluid communication with the first fluid reservoir and the second fluid reservoir and the third fluid reservoir. The reservoir cartridge assembly may also include a pump chamber assembly having a pump chamber with an interior volume which is at least partially bounded by a pump housing, an inlet port in fluid communication with the reservoir selector assembly, a resilient inlet membrane which is disposed adjacent the inlet port, which is spaced from the inlet port when in a relaxed state, and which is sufficiently distendable towards the inlet port to seal the inlet port in a compressed state. The pump chamber assembly may further include an outlet port in fluid communication with the interior volume and with an outlet conduit, a resilient outlet membrane which is disposed adjacent the outlet port, which is spaced from the outlet port when in a relaxed state, and which is sufficiently distendable towards the outlet port to seal the outlet port in a compressed state. In some instances, the outlet port of the pump chamber assembly may be disposed in fluid communication directly with a delivery cannula type of device. The pump chamber assembly may also include a displacement chamber disposed within the interior volume, a resilient displacement membrane which is disposed adjacent the displacement chamber, which forms at least a portion of a boundary of the displacement chamber, which is sufficiently inwardly distendable from a relaxed state to reduce the volume of the displacement chamber when in a compressed state, and which is sufficiently resilient to rebound and increase the volume of the displacement chamber when released from the compressed state. The medical pump system may also include an actuator assembly that is configured to be operatively and releasably coupled to the reservoir cartridge assembly. The actuator assembly may include a cam assembly having an inlet cam lobe which is operatively coupled to the resilient inlet membrane, an outlet cam lobe which is operatively coupled to the resilient outlet membrane, a displacement cam lobe which is operatively coupled to the displacement membrane, and a cam segment which is operatively coupled to the reservoir selector assembly. The actuator assembly may further include a motor operatively coupled to the cam assembly and a controller operatively coupled to the motor.

Some embodiments of a reservoir cartridge assembly which is configured to be operatively and releasably coupled to an actuator assembly of a medical pump system, may include a reservoir base, a first fluid reservoir disposed on the reservoir base, and a second fluid reservoir disposed on the reservoir base. The reservoir cartridge assembly may also have a selector valve assembly of a reservoir selector assembly and a pump chamber assembly secured in fixed relation to the reservoir base. The pump chamber assembly may include a pump chamber having an interior volume which is at least partially bounded by a pump housing, an inlet port in fluid communication with the selector valve assembly of the reservoir selector assembly, a resilient inlet membrane which is disposed adjacent the inlet port, which is spaced from the inlet port when in a relaxed state, and which is sufficiently distendable towards the inlet port to seal the inlet port in a compressed state. The pump chamber assembly may also include an outlet port in fluid communication with the interior volume and with an outlet conduit, a resilient outlet membrane which is disposed adjacent the outlet port, which is spaced from the outlet port when in a relaxed state, and which is sufficiently distendable towards the outlet port to seal the outlet port in a compressed state. The pump chamber assembly may also have a displacement chamber disposed within the interior volume, a resilient displacement membrane which is disposed adjacent the displacement chamber, which forms at least a portion of a boundary of the displacement chamber, which is sufficiently inwardly distendable from a relaxed state to reduce the volume of the displacement chamber when in a compressed state, and which is sufficiently resilient to increase the volume of the displacement chamber when released from the compressed state.

Some embodiments of an actuator assembly which is configured to be operatively and releasably coupled to a reservoir cartridge assembly of a medical pump system may include an actuator chassis, a controller disposed on the actuator chassis, and a cam assembly which is disposed on the actuator chassis and which includes an inlet cam lobe which is configured to be operatively coupled to a resilient inlet membrane, an outlet cam lobe which is configured to be operatively coupled to a resilient outlet membrane, a displacement cam lobe which is configured to be operatively coupled to a displacement membrane, a vent cam lobe which is configured to be operatively coupled to a vent membrane and a cam segment which is configured to be operatively coupled to a selector valve assembly of a reservoir selector assembly. The actuator assembly may further include a motor which is operatively coupled to the cam assembly and the controller. In some instances, the cam segment may be coupled to the cam assembly with a clutch assembly, such as a spring clutch assembly.

Some embodiments of a medical pump system may include a reservoir cartridge assembly having a first fluid reservoir, a second fluid reservoir, a source of filtered air, and a reservoir selector assembly in selective fluid communication with the first fluid reservoir, the second fluid reservoir and the source of filtered air. The medical pump system may also include a pump chamber assembly in fluid communication with the reservoir selector assembly and an actuator assembly that is configured to be operatively coupled to the reservoir cartridge assembly. The actuator assembly may include a cam assembly which is operatively coupled to the pump chamber assembly and a cam segment which is operatively coupled to the reservoir selector assembly. A motor may be operatively coupled to the cam assembly and a controller may be operatively coupled to the motor.

Some embodiments of a reservoir cartridge assembly which is configured to be operatively coupled to an actuator assembly of a medical pump system may include a first fluid reservoir, a second fluid reservoir, a source of filtered air, and a reservoir selector assembly in selective fluid communication with the first fluid reservoir, the second fluid reservoir and the source of filtered air. The reservoir selector assembly may further have a selector valve assembly. A pump chamber assembly may be disposed in fluid communication with the reservoir selector assembly.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

FIG. 1 is a schematic depiction of a medical pump system embodiment.

FIG. 2 is a perspective view of a medical pump system embodiment.

FIG. 3 is an elevation view in partial section of the medical pump system embodiment of FIG. 2 deployed on and releasably secured to a patient’s skin with a distal end of a flexible cannula of a patient port disposed in subcutaneous target tissue.

FIG. 4 is an exploded view of the medical pump system embodiment of FIG. 2.

FIG. 5 is an exploded view of an actuator assembly embodiment of the medical pump system embodiment of FIG. 2.

FIG. 6 is a perspective view of the actuator assembly embodiment of FIG. 5 shown without the outer shell for purposes of illustration.

FIG. 7 is a bottom view of the actuator assembly embodiment of FIG. 6.

FIG. 8 is an exploded view of a reservoir cartridge embodiment of the medical pump system embodiment of FIG. 2.

FIG. 8A is an elevation view in longitudinal section of a pushrod guide of FIG. 8 taken along lines 8A-8A of FIG. 8.

FIG. 8B is a transverse section of the pushrod guide of FIG. 8A taken along lines 8B-8B of FIG. 8A.

FIG. 8C is a longitudinal section of a continuous pump membrane embodiment of FIG. 8 taken along lines 8C-8C of FIG. 8.

FIG. 9 is a perspective view of the medical pump system of FIG. 2 shown without the outer shell for purposes of illustration.

FIG. 10 is a perspective view in transverse section of the medical pump system embodiment of FIG. 2.

FIG. 11 is an enlarged view of the medical pump system embodiment of FIG. 10 indicated by the encircled portion 11-11 in FIG. 10.

FIG. 12 is a top view in perspective of a subassembly including a reservoir base embodiment of the reservoir cartridge assembly and a cam shaft and drive train embodiment of the actuator assembly with the cam shaft of the actuator assembly operatively coupled to the pushrods of the reservoir cartridge assembly.

FIG. 13 is a side view of the subassembly of FIG. 12.

FIG. 14 is a section view of the pump assembly of FIG. 13 taken along lines 14-14 of FIG. 13.

FIG. 14AA is an enlarged view of the reservoir cartridge assembly of FIG. 14 indicated by the encircled portion 14AA-14AA in FIG. 14.

FIGS. 14A-14C are schematic section views of the pump assembly of FIG. 13 illustrating a pumping sequence.

FIG. 15 is a transverse cross section view of the medical pump system embodiment of FIG. 2.

FIG. 16 is an enlarged view of the medical pump system embodiment of FIG. 15 indicated by the encircled portion 16-16 of FIG. 15.

FIG. 17 is an enlarged bottom view partially cut away of the reservoir cartridge assembly embodiment and showing a latch spring embodiment thereof.

FIG. 18 is an enlarged perspective view, partially cut away of the reservoir cartridge assembly embodiment and showing a fill port embodiment thereof.

FIGS. 19A-19C are transverse cross section views of a reservoir cartridge subassembly and illustrating a fill sequence of the fluid volume of a fluid reservoir thereof.

FIG. 20 is a schematic representation of a pump assembly embodiment.

FIG. 21 is a schematic representation of a medical pump training system embodiment.

FIG. 22 is an exploded view of a training cartridge embodiment.

FIG. 23 is a perspective view of the training cartridge embodiment of FIG. 22.

FIG. 24 is a flowchart indicating a method of communication between components of a medical pump training system embodiment.

FIG. 25 is a schematic view of a medical pump system embodiment that includes a plurality of fluid reservoirs.

FIG. 26 is a schematic view of a medical pump system embodiment that includes a plurality of fluid reservoirs.

FIG. 27 is a schematic view of a medical pump system embodiment that includes a plurality of fluid reservoirs.

FIG. 28 is a schematic view of a medical pump system embodiment that includes a plurality of fluid reservoirs.

FIG. 29 is a schematic view of a medical pump system embodiment.

FIGS. 29A and 29B illustrate separation of a first and second therapeutic fluid in an outlet conduit by a bolus of air.

FIG. 30 is a schematic view of a medical pump system embodiment.

FIG. 31 is an exploded view of the medical pump system embodiment of FIG. 30.

FIG. 32 is a perspective view of certain components of the medical pump system embodiment of FIG. 30.

FIG. 33 is a perspective view of certain components of the medical pump system embodiment of FIG. 30.

FIG. 34 is a perspective view of certain components of the medical pump system embodiment of FIG. 30 including a cam assembly, motor and transmission.

FIG. 34A is a perspective view of certain components of the medical pump system embodiment of FIG. 30 including a cam segment and fiducial flag.

FIG. 35 is a perspective view of certain components of the medical pump system embodiment of FIG. 30 including a reservoir base and a flow manifold.

FIGS. 35A-35C illustrate various reservoir selection states of the medical pump system embodiment of FIG. 30.

FIG. 36 is a perspective view of certain components of the medical pump system embodiment of FIG. 30 including a reservoir base, fluid reservoir cover and flow manifold.

FIG. 37 is an elevation view in section of a separated reservoir cartridge assembly and actuator assembly of the medical pump system embodiment of FIG. 30.

FIG. 38 is an elevation view in section of a coupled reservoir cartridge assembly and actuator assembly of the medical pump system embodiment of FIG. 37.

FIG. 39 is an exploded view of a flow manifold embodiment.

The drawings are intended to illustrate certain exemplary embodiments and are not limiting. For clarity and ease of illustration, the drawings may not be made to scale, and in some instances, various aspects may be shown exaggerated or enlarged to facilitate an understanding of particular embodiments.

### DETAILED DESCRIPTION

As discussed above, delivery of therapeutic fluids or non-therapeutic medical fluids is commonly performed intravenously (IV) or subcutaneously using systems that include pumps such as syringe pumps, peristaltic pumps as well as others. However, these types of pumps do not always perform consistently and cost effectively, particularly when used in varying environmental conditions. Medical pump embodiments and related components that address issues such as these are discussed in U.S. Pat. Application No. 16/028,256, filed Jul. 5, 2018, by P. DiPerna et al., titled “Medical Pump with Flow Control”, U.S. Pat. Application No. 16/520,521, filed Jul. 24, 2019, by P. DiPerna et al., titled “Subcutaneous Access Hub with Multiple Cannula Ports”, and U.S. Pat. Application No. 15/122,132, Publication No. US 2016/0361489 A1, filed Mar. 3, 2015, by P. DiPerna, titled “Fluid Delivery Pump”, each of which is incorporated by reference herein in its entirety.

In addition, some or all of these issues may be addressed by improved medical pumping mechanisms that may include a positive displacement pump mechanism. Discussed below are embodiments of micro-positive displacement pump embodiments actuated by a cam assembly that may, in some cases, include a single cam shaft synching an input valve and an output valve of a pump chamber. For some embodiments, such valve embodiments may include the use of one or more diaphragms, also referred to herein as resilient membranes, that are displaced by rotating lobes of the cam assembly. For some embodiments, the lobes of the cam assembly may be rotated by a DC motor coupled through a planetary gearset. Such positive displacement pump embodiments may be incorporated into a medical pump system that includes a reservoir cartridge assembly and a cooperating actuator assembly that may be configured to provide both convenient and economical use for a patient end user of the system.

It should be noted that in many cases, the pump embodiments discussed herein may be operated directly by medical professionals that are treating patients. In many cases, the pump embodiments discussed herein may also be operated directly by individual end users that suffer from a particular medical condition, such as diabetes or any other condition that may require accurate and reliable infusion of a therapeutic fluid. Such individual end users may be using such pump system embodiments to administer therapeutic fluids to themselves under the direction of a medical professional or any other suitable direction. In either case, the person receiving such a treatment will generally be referred to herein as a patient, although the terms end user, patient and the like may be used interchangeably.

For such embodiments, a full revolution of the cam shaft may provide a single fill and dispense cycle for a small volume of fluid from the pump chamber of the medical pump system in some cases. The inlet port and outlet port may be closed by the respective cam shaft lobes by a method wherein upon rotation at a particular phase the respective cam lobe pushes down on an appropriate piston/pushrod element, which may also be referred to herein as a pushrod, to compress the resilient membrane and complete a sealed closure of the port. Timing of the inlet cam lobe and outlet cam lobe may be configured by design such that either the inlet port, outlet port, or both inlet port and outlet port may be closed off at certain phases of the cam lobe rotation. The cam assembly may be configured to sequence the displacement and direction of the pushrods in order to ensure that there is never an open fluid path from a fluid reservoir of a reservoir cartridge assembly to the body of the patient, e.g., via an outlet conduit of the medical pump system to a hub of a patient port that is in fluid communication with a subcutaneous portion of a patient’s body that may include a LuerTM connection to an infusion set or the like. For some such embodiments, there may be four unique states of the pushrods, e.g.: a fill state, a pre-dispense state, a dispense state, and a pre-fill state.

For some embodiments, the motor may be driven through the discharge of a capacitor which can also be useful to reduce the risk of a continuous runaway condition for the motor. The motor rotation speed may be controlled by pulsing a discharge of such a capacitor. The frequency of discharged pulses may be controlled by embedded firmware which may be configured to support partial pumping cycles, partial dispense cycles or the like. An electrical switch such as a micro-switch may be positioned onto a shaft of the cam assembly to confirm a proper or otherwise desired rotation state of the cam shaft and/or motor shaft with respect to output steps of the motor.

In some cases, a pressure sensor disposed in the actuator assembly may be configured to interface with the reservoir cartridge assembly and used to determine a pressure within an air volume of the fluid reservoir of the reservoir cartridge assembly. In some cases, the pressure sensor may be used to measure a pressure differential within the air volume when fluid is drawn from the liquid volume of the fluid reservoir to fill the pump chamber thereby reducing a volume of liquid disposed in the liquid volume. Such pressure readings may also be used to provide increased sensitivity for detecting occlusions in a fluid path of the outlet port, or outlet conduit, such as the infusion tubing, between the outlet port and hub of the infusion set.

Such a medical pump system or components thereof may be useful for delivery of non-therapeutic fluids or therapeutic fluids such as saline, antibiotics, dextrose solutions, pain medications, peptides and the like. Some therapeutic fluids that may be delivered by the medical pump system embodiments discussed herein may include therapeutic fluids used for the treatment of diabetes as well as other related medical conditions. In particular, such medical pump systems or components thereof may be useful for the continuous subcutaneous delivery of insulin, including standard insulin compositions such as Novolog®, Lyumjev™, Fiasp®, and Humalog®, fast-acting insulins such as Lispro, Aspart, and Glulisine, and slow-acting insulin compositions such as insulin Glargine and insulin Detemin. Other therapeutic fluids used for the treatment of diabetes or any other suitable medical condition where accurate and cost effective delivery of fluids to a patient is needed such as liquid stable glucagon, pramlintide, SGLT-2 and GLP-1 may also be delivered. Such medical pump systems may be particularly useful where such fluid delivery is being carried out in varying environmental conditions and/or where ambulatory delivery is desirable.

For some medical pump system embodiments, cost effectiveness and efficiency may be realized by identifying a first set of components that may be included with a durable element and a second set of components that may be included with a low use or disposable single use type element of a medical pump system. For such embodiments, the more costly and/or more durable components may be included with the first set of components of the durable element in order to reuse and make efficient use of these types of components. Less expensive components, components that require frequent refreshing and/or those components that require sterilization before each use may be incorporated into the second set of components of the disposable element.

As such, for some medical pump system embodiments discussed herein, the motor, transmission, cam or cams with a clutch assembly, sensitive pressure sensor, and controller which may include a microprocessor and memory may be included in a reusable actuator assembly that may be categorized as the durable element, although any other suitable combination of components or elements may be used for the durable actuator assembly. Components such as a fluid reservoir, pump chamber and its associated elements and a power source such as a battery may be included in the reservoir cartridge assembly which may be categorized as the less durable or disposable element, although any other suitable combination of elements may also be used for reservoir cartridge assembly embodiments. Additional sub-assemblies may include a mount bracket that is configured to detachably mount the medical pump assembly to the patient’s body with a single use adhesive pad that is generally serviceable for about 1 day to about 6 days, a service life that may be similar to the service life of embodiments of the reservoir cartridge assembly. In some cases, the durable element of the actuator may have a service life of up to about 6 months or more.

Pump assembly embodiments discussed herein may be configured to reduce or eliminate the possible detrimental effects of harsh and/or sudden mechanical movements upon the molecules of certain therapeutic fluids such as insulin. As such, the device and method embodiments for fluid delivery discussed herein are consistent with the cam lobes of the cam assembly embodiments rotating slowly (in some cases up to only about two revolutions per minute during the fastest bolus delivery) allowing the cam lobes to gently open and close respective ports controlled thereby so as to move the molecules of the therapeutic fluid through the pump assembly embodiments without damage to the molecules of the therapeutic fluids, such as for example insulin molecules.

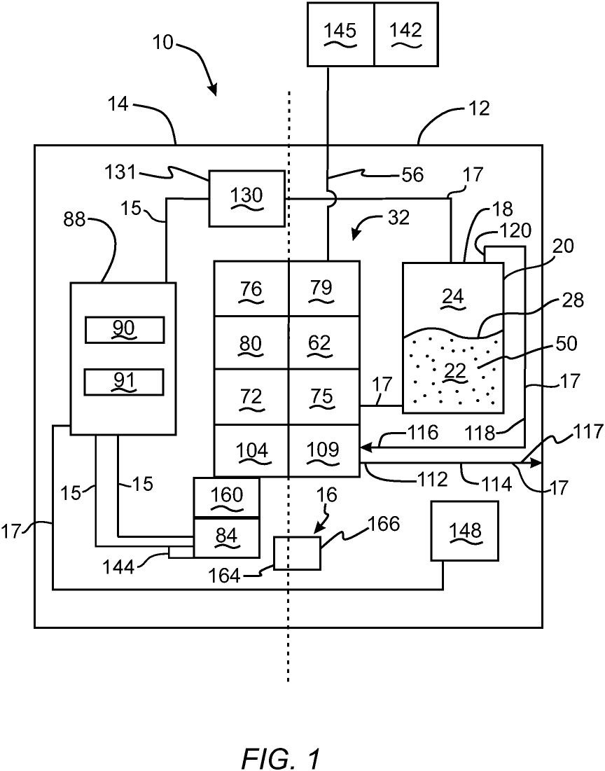

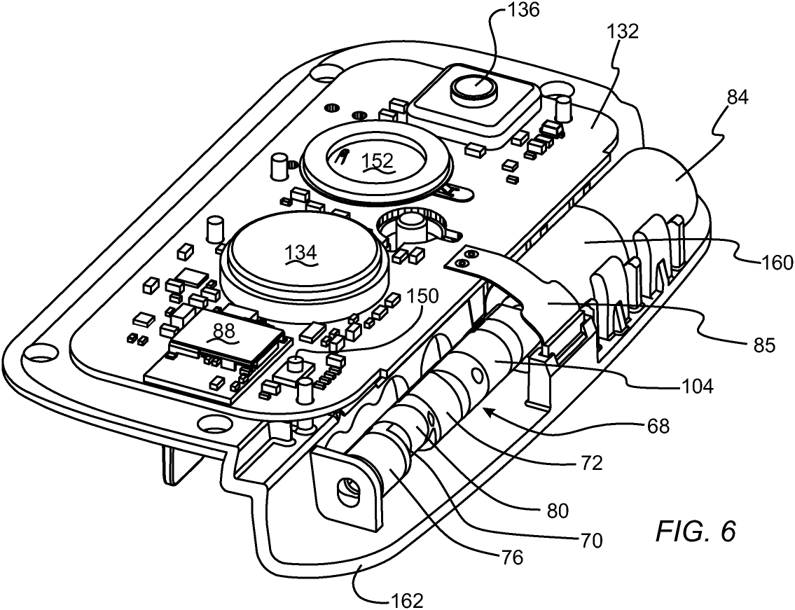

Referring generally to FIGS. 1-10, a medical pump system embodiment 10 is shown that includes two major components including a reservoir cartridge assembly 12 and an actuator assembly 14 that are configured to be coupled together with a latch mechanism 16 that holds them securely together, but that can later be released to install a new reservoir cartridge assembly 12 into the reusable actuator assembly 14. A schematic overview of an embodiment of the medical pump system 10 is shown in FIG. 1 wherein a dashed line indicates a coupling interface between the actuator assembly 14 and the reservoir cartridge assembly 12, and the various components thereof. The interconnecting lines between various schematic components of FIG. 1 may be conducting conduits 15 and include any type of suitable conduit that may be useful for operatively interconnecting the respective components such as information conducting conduits, power conducting conduits or the like including conductive wires, optical fibers, wireless connectivity etc. In addition, fluid conduits 17 may be used to interconnect and provide fluid communication between volumes or fluid pathways of the medical pump system 10 and may include channels, tubes, pipes or any other appropriate type of internal volume or lumen.

In general, for some embodiments, the actuator assembly embodiment 14 may be a durable element that may be used over several months or more and the reservoir cartridge assembly 12 may be a disposable single use element that is replaced on a more frequent basis, such as every few days. In addition, although the various medical pump system embodiments discussed herein are shown with the respective reservoir cartridge assembly embodiments 12 and actuator assembly embodiments 14 including particular elements or components, these illustrative embodiments are not meant to be limiting any other suitable combination of components or elements discussed herein may be used for either the reservoir cartridge assembly 12 or actuator assembly 14. The medical pump system embodiments 10 discussed herein are suitable for ambulatory use and may have outer dimensions suitable for such use. In some cases, embodiments of the medical pump systems 10 discussed herein may have a length of about 2.0 inches to about 3.0 inches, a width of about 1.2 inches to about 1.8 inches and a thickness of about 0.4 inches to about 0.7 inches.

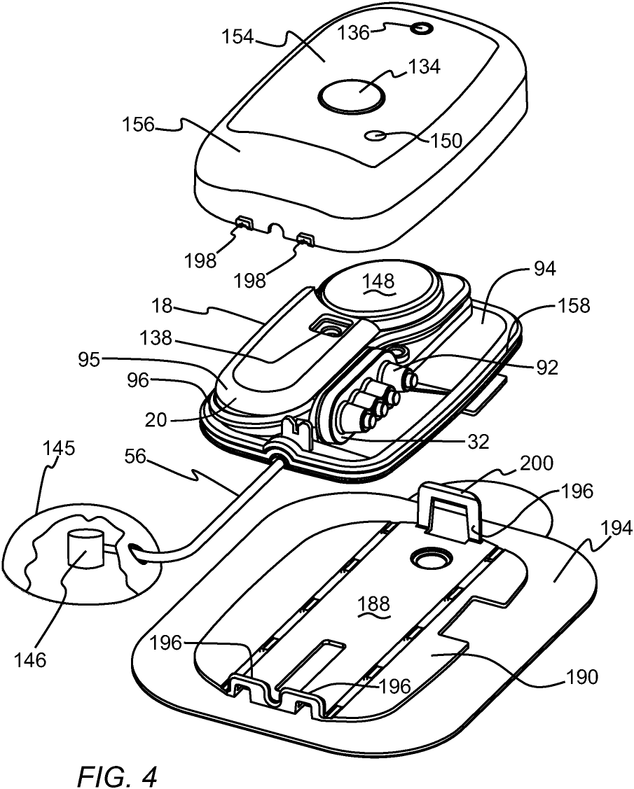

In some cases, the reservoir cartridge assembly 12 may include a fluid reservoir 18 as shown in FIGS. 1 and 4 which may have an outer structure or container that is rigid and resistant to flexing in response to pressure differentials imposed between an inner volume thereof and the area disposed outside of the outer structure or container. Within this rigid outer container 20, in some cases, the fluid reservoir 18 may also have a liquid volume 22, an air volume 24 and a flexible membrane 28 disposed between the liquid volume 22 and air volume 24. The flexible membrane 28 may be made from a fluid tight material and thus provides a fluid tight barrier between the air volume 24 and liquid volume 22. For some embodiments, the flexible membrane 28 may be molded by methods such as cold forming, pressure /vacuum forming and the like in order to conform to the inner contour shape of the fluid reservoir cavity of a fluid reservoir base 94 or fluid reservoir cover 95 which may be configured to form the rigid outer container 20. The outer perimeter of the flexible membrane may be sealingly secured to the outer perimeter of the fluid reservoir cavity of the reservoir base 94 by methods such as heat sealing, adhesive bonding or the like.

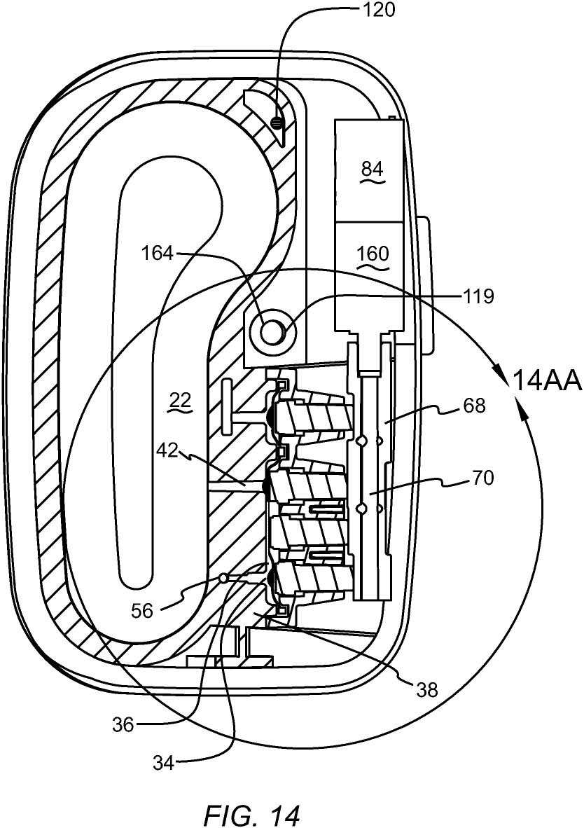

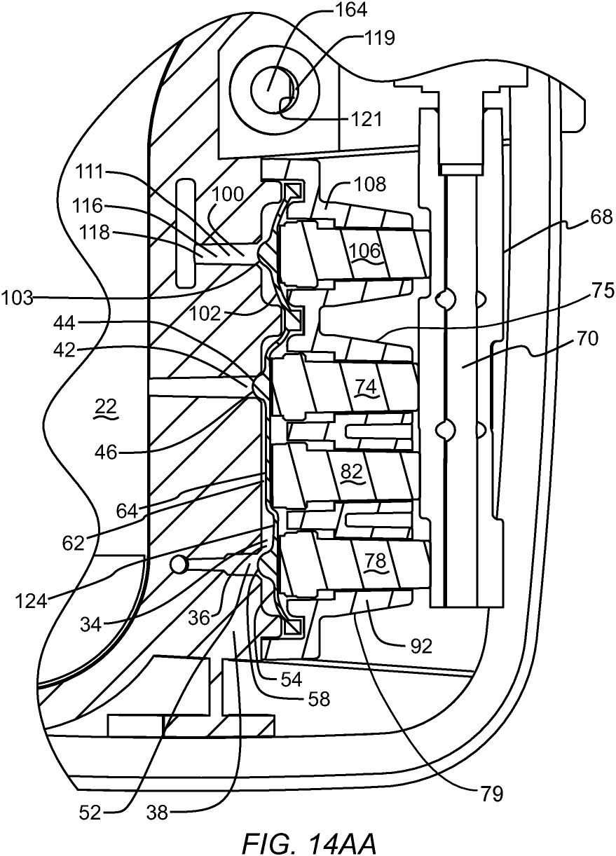

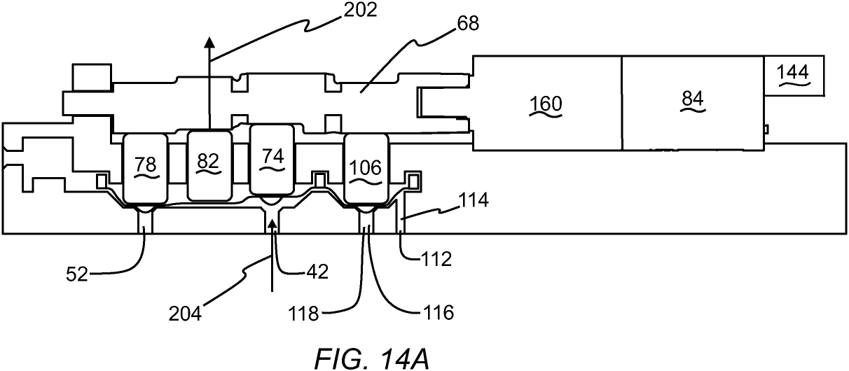

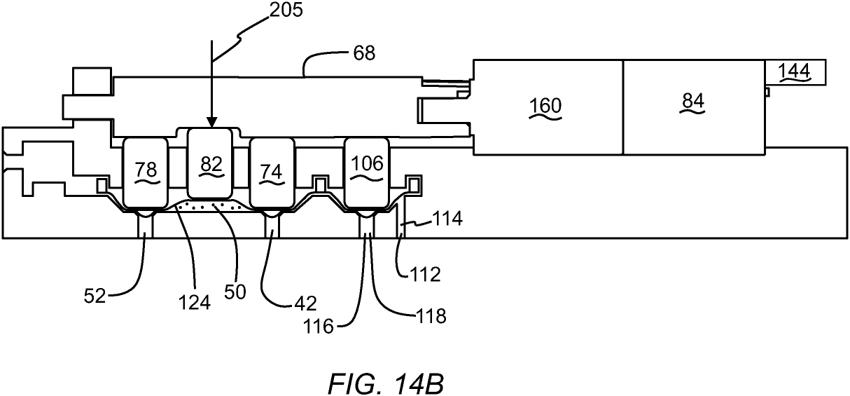

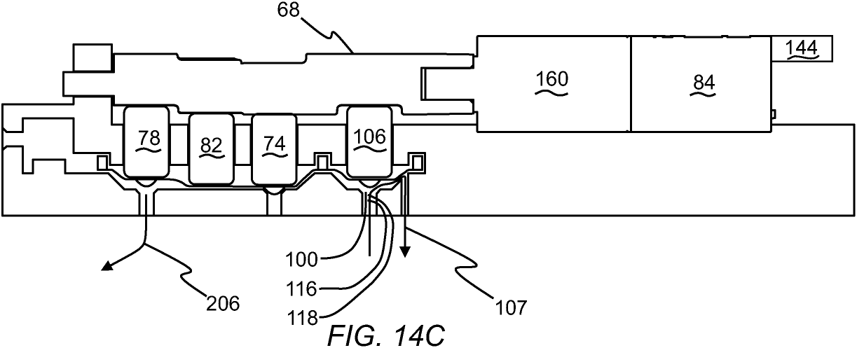

A pump chamber assembly 32, as also seen in FIG. 4, of the reservoir cartridge assembly 12 may include a pump chamber 34, as shown in FIGS. 14 and 14AA, having an interior volume 36 which is at least partially bounded by a pump housing 38. An inlet port 42 of the pump chamber assembly 32 is disposed in fluid communication with the interior volume 36 of the pump chamber 34 and also with the liquid volume 22 of the fluid reservoir 18 which allows therapeutic fluid 50 disposed in the liquid volume 22 of the fluid reservoir 18 to flow into the pump chamber 34 when the inlet port 42 is open. A resilient inlet membrane 44 is disposed adjacent the inlet port 42 and is spaced from the inlet port 42 when in a relaxed state without any external force being applied to it. The resilient inlet membrane 44 is also sufficiently distendable towards the inlet port 42 to seal the inlet port when the resilient inlet membrane 44 is in a compressed state biased towards the inlet port 42. The resilient inlet membrane 44 may also include a dimple 46 that aligned with and disposed towards the inlet port 42 and configured to help seal the inlet port 42 when the resilient inlet membrane 44 is pressed into the inlet port 42.

An outlet port 52 is disposed in fluid communication with the interior volume 36 of the pump chamber 34 and is also disposed in fluid communication with an outlet conduit 56 which allows therapeutic fluid 50 to flow out of the outlet port 52 from the pump chamber 34 when the outlet port 52 is open. A resilient outlet membrane 54 is disposed adjacent the outlet port 52 and is spaced from the outlet port 52 when in a relaxed non-distended state. The resilient outlet membrane 54 is also sufficiently distendable towards the outlet port 52 to seal the outlet port 52 when in a compressed state distended towards the outlet port 52. The resilient outlet membrane 54 may also include a dimple 58 that aligned with and disposed towards the outlet port 52 and is configured to help seal the outlet port 52 when the resilient outlet membrane 54 is pressed against the outlet port 52. A displacement chamber 62 is also disposed within the interior volume 36 of the pump chamber 34. A resilient displacement membrane 64 is disposed adjacent the displacement chamber 62 and forms at least a portion of a boundary of the displacement chamber 62.

The resilient displacement membrane 64 may also be sufficiently inwardly distendable from a relaxed state to reduce the volume of the displacement chamber 62 when in a compressed state distended inwardly towards the opposite wall of the interior volume 36 of the pump chamber 34. The resilient displacement membrane 64 is also sufficiently resilient to rebound and increase the volume of the displacement chamber 62 when released from the compressed state thereby moving away from the wall opposite the resilient displacement membrane 64. In general, the resilient inlet membrane 44, resilient outlet membrane 54 and resilient displacement membrane 64 may be distended, compressed, and relaxed by the actuation of respective pushrods with valve ends thereof disposed in contact with the resilient membranes 44, 54, 64 discussed in more detail below.

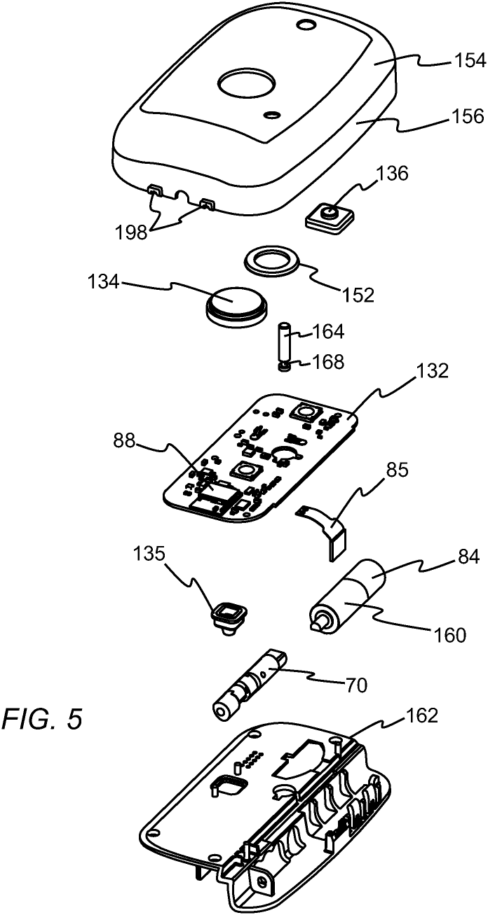

The pushrods in contact with the various resilient membranes 44, 54, 64 of the pump chamber assembly 32 may be actuated by a cam assembly 68 of the actuator assembly 14. For some embodiments, the actuator assembly 14 may be configured to be operatively and releasably coupled to the reservoir cartridge assembly 12 as noted above. Embodiments of the actuator assembly 14 may include the cam assembly 68 which may have a cam shaft 70 with an inlet cam lobe 72 which is operatively coupled to the resilient inlet membrane 44, an outlet cam lobe 76 which is operatively coupled to the resilient outlet membrane 54, and a displacement cam lobe 80 which is operatively coupled to the resilient displacement membrane 64. The actuator assembly 14 may also include a motor 84 operatively coupled to the cam assembly 68 and a controller 88 operatively coupled to the motor 84. In some cases, the motor 84 may be coupled to the controller 88 with a flexboard assembly conduit such as the flexboard assembly conduit embodiment 85 as seen in FIG. 5 for example. However, the motor 84 may also typically be operatively coupled to the controller 88 with conductive conduits 15 such as wire and a connector.

For some embodiments, the reservoir cartridge assembly 12 may further include an inlet pushrod 74 which is operatively disposed between the inlet cam lobe 72 and the resilient inlet membrane 44, an outlet pushrod 78 operatively disposed between the outlet cam lobe 76 and the resilient outlet membrane 54 and a displacement pushrod 82 operatively disposed between the displacement cam lobe 80 and the resilient displacement membrane 64. A pushrod guide 92 may be secured to the reservoir base 94 of the reservoir cartridge assembly 12. Such a pushrod guide may include a rigid configuration with an inlet pushrod bore disposed about and guiding the inlet pushrod 74, a displacement pushrod bore disposed about and guiding the displacement pushrod 82 and an outlet pushrod bore disposed about and guiding the outlet pushrod 78.

Regarding the respective inlet and outlet valve assemblies discussed above, a combination of the inlet port 42, resilient inlet membrane 44, inlet pushrod 74 and associated portion of the pushrod guide 92 may be said to form an inlet valve assembly 75 as illustrated in FIGS. 1 and 14AA. A combination of the outlet port 52, resilient outlet membrane 54, outlet pushrod 78 and associated portion of the pushrod guide 92 may be said to form an outlet valve assembly 79. In addition, with regard to this configuration, the resilient inlet membrane 44 may be said to be operatively coupled to the inlet cam lobe 72 by the inlet pushrod 74, the resilient outlet membrane 54 may be said to be operatively coupled to the outlet cam lobe 76 by the outlet pushrod 78, and the resilient displacement membrane 64 may be said to be operatively coupled to the displacement cam lobe 80 by the displacement pushrod 82.

In some cases, the reservoir cartridge assembly 12 may further include a vent port 100 and a resilient vent membrane 102 which is disposed adjacent the vent port 100 and which is also spaced from the vent port 100 when in a relaxed state as shown in FIG. 14AA. The resilient vent membrane 102 is also sufficiently distendable towards the vent port 100 to seal the vent port 100 when the resilient vent membrane 102 is in a compressed state distended towards the vent port 100. In addition, the cam assembly 68 may further include a vent cam lobe 104 which is operatively coupled to the resilient vent membrane 102 by a vent pushrod 106 which is operatively disposed between the vent cam lobe 104 and the resilient vent membrane 102.

In addition, a vent pushrod guide portion 108 of the pushrod guide 92 may be secured in fixed relation to the pump housing 38 and include a vent pushrod bore 110 disposed about and guiding the vent pushrod 106. A combination of the vent port 100, resilient vent membrane 102, vent pushrod 106 and vent pushrod guide portion 108 may be said to form a vent valve assembly 109 as shown in FIG. 1. The resilient vent membrane 102 may also include a dimple 103 that is aligned with and disposed towards the vent port 100 and configured to help seal the vent port 100 when the resilient vent membrane 103 is pressed into the vent port 100.

In some cases, the state of the vent valve assembly 109 may determine whether the air volume 24 of the fluid reservoir 18 is vented to the ambient atmosphere or not. In particular for some embodiments, if the vent port 100 of the vent valve assembly 109 is closed, as shown in FIG. 14A, then the air volume 24 is not vented to the ambient atmosphere outside of the medical pump system 10. If the vent port 100 of the vent valve assembly 109 is open, as shown in FIG. 14C, then the air volume 24 is vented to the ambient atmosphere through the vent valve assembly 109 as indicated by the arrow 107. As such, the vent valve assembly 109 acts as a gateway for a vent conduit pathway 111 as shown in FIG. 14AA that extends from the air volume 24 to the ambient atmosphere disposed outside the medical pump system 10.

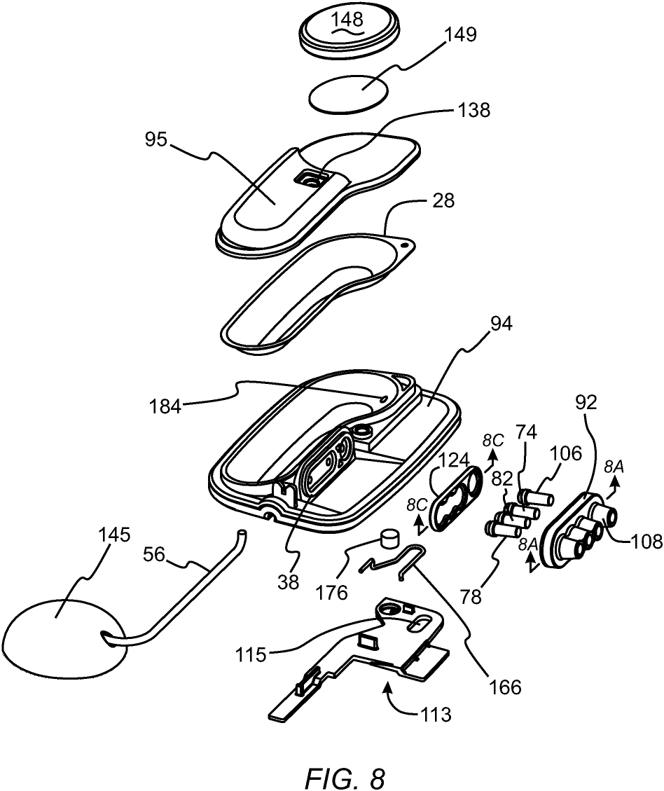

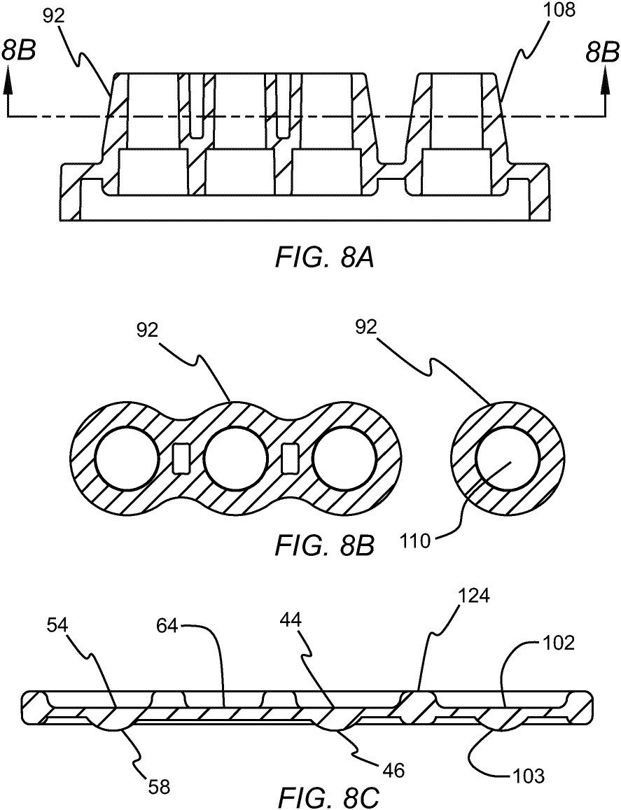

For some embodiments, the pushrod guide 92 and pushrod bores disposed therein may be configured such that each longitudinal axis of the respective pushrod bores, including the inlet pushrod bore, outlet pushrod bore, displacement pushrod bore and vent pushrod bore, are all parallel to each other and may also all lie in a common plane as shown in the embodiment of FIGS. 8, 8A and 8B. As such, the pushrods associated with this configuration, including the inlet pushrod 74, outlet pushrod 78, displacement pushrod 82 and vent pushrod 106, may also have respective longitudinal axes that are all parallel to each other and lie in a common plane when assembled in the pushrod guide 92. The pushrods including the inlet pushrod 74, outlet pushrod 78, displacement pushrod 82 and vent pushrod 106, may have a generally cylindrical configuration with a flanged portion disposed at an inward end of the pushrod. The flanged portion may extend radially outward so as to be mechanically captured by a corresponding expanded section of each respective pushrod bore, with each expanded section also being disposed at the inward end of the pushrod guide 92.

In such cases, the flanged portion of each pushrod may be small enough to fit and slide easily within the expanded section of its respective pushrod bore, but too large in transverse dimension to fit into the nominal bore. The axial length of the expanded section of each pushrod bore may be sufficiently greater than an axial length of each respective flanged portion such that each pushrod which is slidingly disposed within its pushrod bore of the pushrod guide 92 may be configured to slide in an axial direction within the pushrod bore over a limited axial range determined by the axial length of the expanded section. For this linearly oriented configuration, the associated pump chamber 34 may also be similarly configured with the shallow elongate interior volume 36 of the pump chamber 34 having the inlet port 42, displacement chamber 62 and outlet port 52 lying along a line with the displacement chamber 62 disposed between the inlet port 42 and outlet port 52.

For some embodiments, the pump chamber 34 may be configured as a shallow rectangular shape with radiused ends bounded on one side by the pump chamber housing 38 and on the other side by the resilient continuous pump membrane 124 as seen in FIG. 8C. The inlet port 42 and outlet port 52 are formed into the pump chamber housing 38. Other such pump chamber embodiments 34 may also have other suitable configurations wherein the inlet port 42, outlet port 52 and displacement chamber 62 do not all lie along the same line, nor would the associated resilient membranes and associated pushrods. The linear configuration of the embodiment shown may be useful for pump assemblies that utilize a single linear cam shaft 70 that includes the multiple lobes associated with each portion of the pump chamber 34 and/or vent valve assembly 109.

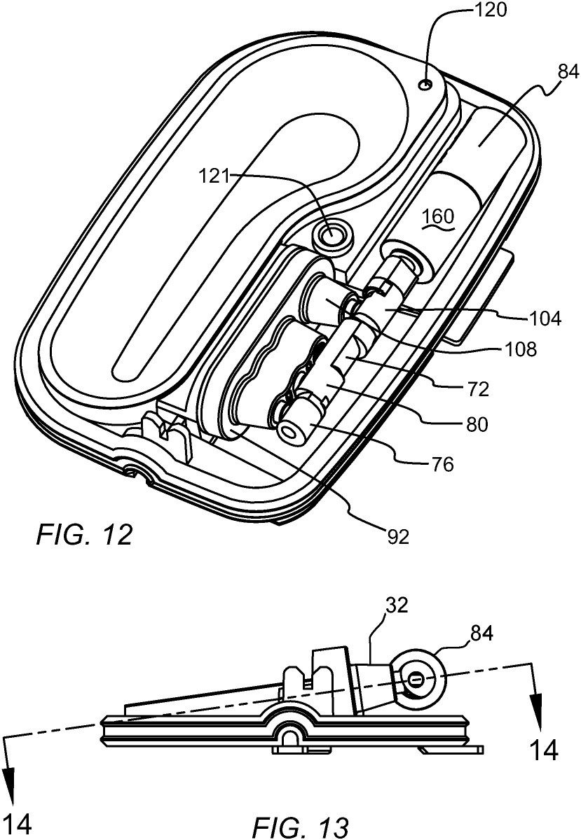

For some embodiments, the vent valve assembly 109, and particularly an inner volume thereof, may be disposed in fluid communication with an outlet end 116 of a pre-valve vent conduit 118 of the vent conduit pathway 111 as shown in FIGS. 14A-C, and 19A-B. The pre-valve vent conduit 118 extends from the vent valve assembly 109 to an inlet end 120 of the pre-valve vent conduit 118 as shown in FIGS. 12 and 14, with the inlet end 120 being disposed adjacent and in fluid communication with the air volume 24 of the fluid reservoir 18. The pre-valve vent conduit 118 extends through the reservoir cartridge assembly 12 through a channel formed by a slot in the reservoir base 94 that is sealed on a bottom side thereof by an upper surface of a latch spring cover plate 113 as shown in FIGS. 8, 18, 19A and 19B. The latch spring cover plate 113 also includes a latch release slot 115 that provides limited access for release of the latch spring 166 discussed in more detail below. The latch release slot 115 may also serve as the final access point to ambient atmosphere for the vent conduit pathway 111 or may optionally be covered with a gas permeable membrane to add air that has been filtered.

The vent valve assembly 109, and particularly the inner volume thereof, may also be disposed in fluid communication with an inlet end 112 of a post-valve vent conduit 114 of the vent conduit pathway 111, as shown in FIGS. 14A-B and 19A-B. The post-valve vent conduit 114 is also in fluid communication with the ambient atmosphere that surrounds the medical pump system 10 at an outlet end 117 thereof. The vent path of the post-valve vent conduit 114 begins at the inlet end 112 adjacent the vent valve assembly 109 and then vents into an interior volume space disposed between the reservoir cartridge assembly 12 and the actuator assembly 14, this interior volume space being sealed around a perimeter thereof as discussed in more detail below. The vent path then continues to an air gap 119 disposed between an outside surface of a latch post 164 that is secured to an actuator chassis 162 and a latch post bore 121 disposed in the reservoir base 94 of the reservoir cartridge assembly 12.

The latch post 164 and latch post bore 121 are components of the latch mechanism 16 discussed in more detail below. The air gap 119 and latch post bore 121 are shown in FIGS. 8, 12, and 14AA. The vent path then continues from the air gap 119 to the latch release slot 115 disposed on the bottom of the reservoir cartridge assembly 12 and then out to ambient atmosphere. For embodiments such as these, the latch release slot 115 may serve as the outlet end 117 of the post-valve vent conduit 114. The tortuous nature of the vent conduit pathway 111 that includes the pre-valve vent conduit 118 and the post-valve vent conduit 114 may be useful in some circumstances in order to reduce the intrusion of contaminants into the vent conduit pathway 111 as well as the vent valve assembly 109 and air volume 24. In addition, as the vent conduit pathway 111 is included within the reservoir cartridge assembly 12, which may, in some cases, be disposable and used for only a limited amount of time, there is also a limited amount of time for contaminants such as dust, moisture etc. to accumulate in the vent conduit pathway 111.

In some cases, a single continuous pump membrane 124, as shown in FIGS. 8, 8C and 14AA , may be configured to be elastically resilient and to include the resilient inlet membrane 44, resilient displacement membrane 64, and resilient outlet membrane 54. In some instances, this single continuous pump membrane 124 may also include the resilient vent membrane 102. Although, in some cases, each of the inlet cam lobe 72, outlet cam lobe 76, displacement cam lobe 80 and vent cam lobe 104 may be actuated by a separate cam and motor mechanism, in general the inlet cam lobe 72, outlet cam lobe 76, and displacement cam lobe 80 may be disposed on the cam shaft 70 which may have a continuous unitary configuration wherein all of the cam lobes disposed thereon are secured in fixed relation to each other and rotate together.

In addition, for some such integrated cam shaft embodiments 70, the vent cam lobe 104 may also be included and disposed in fixed relation to the inlet cam lobe 72, outlet cam lobe 76 and displacement cam lobe 80 and rotate together with those cam lobes. For this type of unitary cam configuration, the inlet cam lobe 72, outlet cam lobe 76 and displacement cam lobe 80 may be configured and phased to generate a pumping cycle with each rotation of the cam shaft, with each pumping cycle including a fill cycle of the pump chamber 34 that includes opening the inlet port 42 while the outlet port 52 is closed, expansion of the displacement chamber 62 while the inlet port 42 is open and then closing the inlet port 42 when the displacement chamber 62 is full of therapeutic fluid 50 while the outlet port 52 is still closed. When the displacement chamber 62 is full and the inlet port 42 and outlet port 52 are both closed, the pump chamber assembly 32 may be said to be in a pre-dispense state.

To carry out this fill cycle, as the cam shaft 70 is being rotated, opening the inlet port 42 includes retracting a contact surface of the inlet cam lobe 72 and associated inlet pushrod 74 to allow the resilient inlet membrane 44 to relax away from the inlet port 42. The outlet port 52 is closed due to the extension of a contact surface of the outlet cam lobe 76 against a cam end of the outlet pushrod 78 which in turn distends the resilient outlet membrane 54 against the outlet port 52 so as to close the outlet port 52. In this case, with the inlet port 42 in an open state, the outlet port 52 in a closed state, and the displacement chamber 62 in a minimum volume state, the pump chamber assembly may be said to be in a pre-fill stage of a pumping cycle. For the next step, the displacement chamber 62 may expanded by retracting a contact surface of the displacement cam lobe 80 and thereby retracting the associated displacement pushrod 82 to allow the resilient displacement membrane 64 to rebound and expand the effective volume of the displacement chamber 62 thus carrying out the fill cycle. For some embodiments, the amount of time for filling the displacement chamber may be about 5 seconds to about 30 seconds or more. In some cases, the displacement chamber 62 may be filled during a fill cycle over a period of about 12 seconds to about 20 seconds or more.

The contact surface of each of the respective cam lobes 72, 76, 80, 104 is that part of the cam lobe that is in contact with the respective pushrod. As such, the respective contact surfaces move around each of the cam lobes as the cam shaft is rotated. It should be noted that in some cases, each of the resilient membranes 44, 54, 64, 102 may be configured such that they are continually applying back pressure to the respective pushrods such that the pushrods are always exerting some pressure against the cam lobes without any lash therebetween. This same arrangement is also present for the single continuous pump membrane embodiment 124 that includes each of the resilient inlet membrane portion 44, resilient outlet membrane portion 54, resilient displacement membrane portion 64 and resilient vent membrane portion 102.

With regard to certain use embodiments of the pump chamber assembly 32 of the medical pump system 10, for some embodiments the inlet cam lobe 72, outlet cam lobe 76 and displacement cam lobe 80 may be configured and phased to generate a dispense cycle that includes opening the outlet port 52 while the inlet port 42 is closed, compression of the displacement chamber 62 while the outlet port 52 is open and closing of the outlet port 52 while the inlet port 42 is still closed. In some cases the inlet cam lobe 72, outlet cam lobe 76 and displacement cam lobe 80 may be configured and phased such that the inlet port 42 and outlet port 52 are never open at the same time during a complete rotation of the cam shaft 70. As such, prior to the initiation of this dispense cycle, the inlet valve 42 is typically closed prior to opening of the outlet valve 52 such that the pump chamber assembly 32 is in a pre-dispense state with the dispense chamber 62 full of therapeutic fluid 50 and both the inlet valve 42 and outlet valve 52 in a closed state.

For some pump assembly embodiments of the medical pump system 10 the volume and configuration of the pump chamber 34 and the lift and duration of the inlet cam lobe 72, outlet cam lobe 76 and displacement cam lobe 80 may be configured to deliver about 2 microliters to about 10 microliters, more specifically, about 4 microliters to about 6 microliters, of therapeutic fluid 50 from the outlet port 52 for each pumping equivalent to one rotation of the cam shaft 70.

With regard to a venting function wherein the vent port 100 is opened to ambient atmosphere such that the air volume 24 of the fluid reservoir 18 is thereby vented to ambient atmosphere through the open vent port 100, in some cases the inlet cam lobe 72, outlet cam lobe 76 and vent cam lobe 104 may be configured and phased such that the vent port 100 is open while the outlet port 52 is open and the inlet port 42 is closed. In some cases, the inlet cam lobe 72, outlet cam lobe 76 and vent cam lobe 104 may be configured and phased such that the vent port 100 is open while the cam shaft 70 is paused after a dispense cycle and before the beginning of a fill cycle. When the vent port 100 is open to ambient atmosphere, a pressure sensor 130 disposed on the actuator assembly 14 and disposed in fluid communication with the air volume 24 of the liquid reservoir 18 may also be exposed to the ambient atmosphere and is thereby configured to monitor the pressure of the ambient atmosphere and sense any changes in the ambient atmospheric pressure during this period.

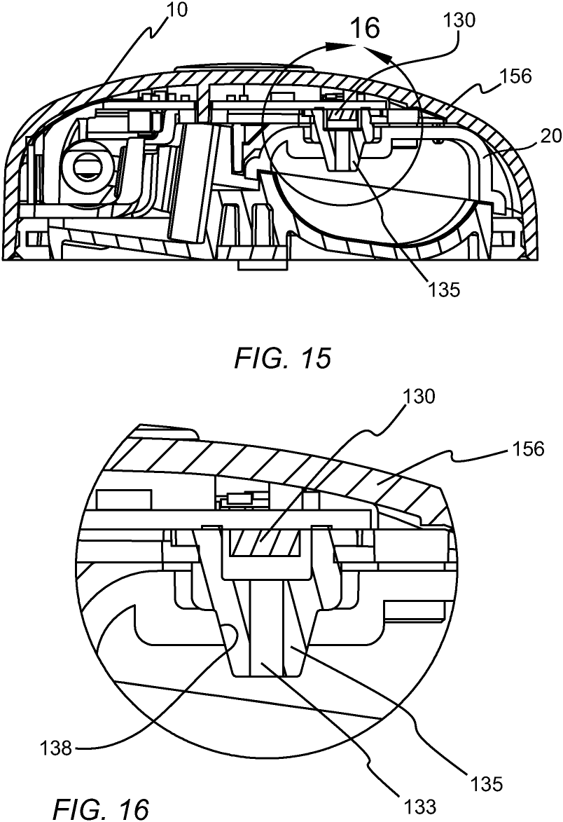

The pressure sensor 130, as shown in FIGS. 1 and 15, may also be configured to determine a remaining volume of therapeutic fluid 50 disposed in the liquid volume 22 of the fluid reservoir 18 by measuring small pressure drops in the air volume 24 during a fill cycle or at any other suitable period of a pumping sequence embodiment. Certain embodiments of the pressure sensor 130 may also include temperature measurement capabilities. For some embodiments, such a pressure sensor 130 may include a software controlled, high performance MEMS nano-pressure sensor having a measurement range of about 260 hPA to about 1260 hPA absolute pressure and a temperature measurement range of about -40° F. to about 180° F.

Because such pressure sensor embodiments 130 are preferably reused and not included in the limited use element of the reservoir cartridge assembly 12, it is necessary to establish a reliable sealed fluid communication path between the pressure sensor 130 of the actuator assembly 14 and the air volume 24 of the reservoir cartridge assembly 12. In some cases, the actuator assembly 14 may include a pressure conduit 133 which is disposed in fluid communication with the pressure sensor 130 and the air volume 24 when the actuator assembly 14 and reservoir cartridge assembly 12 are coupled together. For such an arrangement, the actuator assembly may further include a pressure conduit boot 135 which is secured in fluid communication with the pressure conduit 133 and which is configured to sealingly couple to a boot receptacle 138 as shown in FIGS. 15 and 16. The pressure conduit boot 135 may have a generally tapered shape and be made from a compliant elastic material that will readily and sealingly conform to the boot receptacle 138 as shown in FIGS. 15 and 16. The compliant elastic material of the pressure conduit boot 135 may be configured to repeatably and reliably form a seal with boot receptacle embodiments.

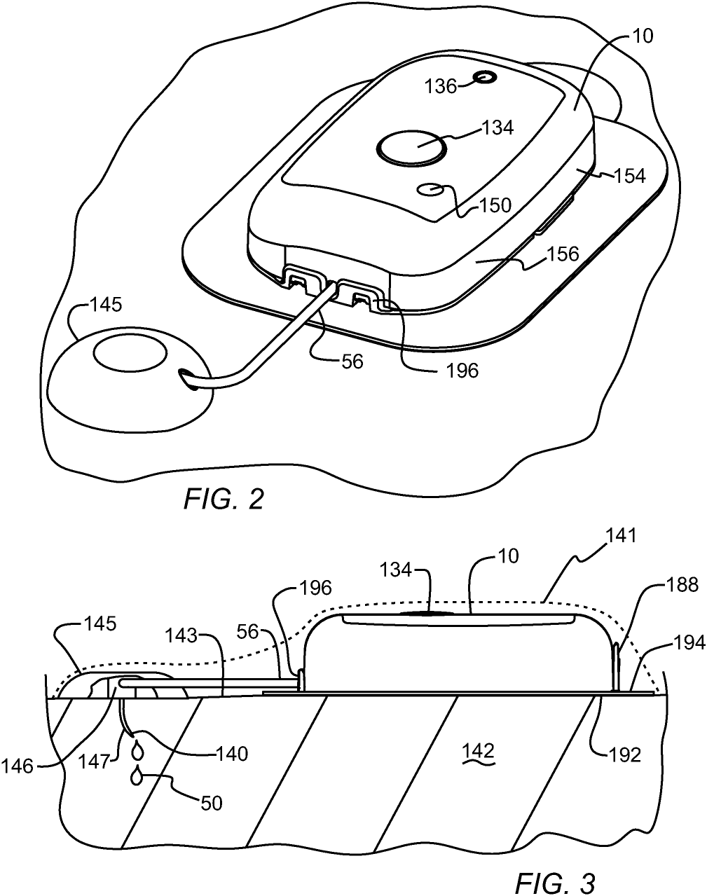

For some embodiments, the actuator assembly 14 may include a printed circuit board (PCB) 132 and the controller 88 may be operatively coupled and otherwise secured to the printed circuit board 132. The controller 88 may include a processor 90 such as a microprocessor, memory 91 as well as any suitable components that may be useful for interfacing with the pressure sensor 130, motor 84, user interface embodiments such as a control button 134, an optional priming button 136 and the like as shown in FIG. 2. Such components may include electrical contacts, electrical conduits such as wiring, as well as drivers and any other machine-readable instructions stored in the memory 91 that may facilitate use of the medical pump system 10. For some embodiments, the controller 88 may include a “system on a chip” type microprocessor, including a low power consuming high performance microprocessor that may support low energy blue tooth, near field communication and the like such as model nRF52832 manufactured by Nordic Semiconductors located in Trondheim, Norway.

With regard to control of the motor 84 and pump chamber assembly 32, in some cases, the controller 88 may be configured to limit the angular velocity of the cam shaft 70 during a dispense cycle to an angular velocity that will generate a maximum flow of up to about 0.5 to about 1.0 microliters (µl) per second through a therapeutic fluid dispense circuit of the reservoir cartridge assembly 12. For some embodiments, the angular velocity of the cam shaft 70 may be limited during a dispense cycle to about 0.25 revolutions per minute to about 3 revolutions per minute. Such a limit on flow velocity of the therapeutic fluid 50 through the various conduits of the medical pump system 10 may be useful in maintaining the integrity of the molecular structure of certain therapeutic fluids 50. In addition, in some instances, the controller 88 may be configured to actuate the motor 84 so as to rotate the cam shaft 70 in distinct rotation steps and take pressure measurements within the air volume 24 of the fluid reservoir 18 between the distinct rotation steps. In some cases, the motor 84 may include a direct current (DC) type electric motor that is coupled to the cam shaft 70 through the transmission 160 which provides gear reduction between rotation of the output shaft of the motor 84 and rotation of the cam shaft 70. In some cases, the gear reduction ratio provided by the transmission 160 may be a gear reduction ratio of about 100:1 to about 500:1, more specifically, about 110:1 to about 130:1.

For such an arrangement, the controller 88 may be configured to generate a small pulse of electricity discharged from a capacitor which may be disposed on the PCB 132 which is communicated to the DC input of the motor 84 so as to generate a pulse of rotation in the drive shaft of the motor and a corresponding pulse of rotation, reduced by the gear reduction of the transmission, in the cam shaft 70. In some instances, such pulses of electricity generated by the controller 88 may be about 5 milliseconds to about 50 milliseconds in duration. Such pulses of drive electricity to the motor 84 may generate rotation pulses of the cam shaft 70 of about 3 degrees to about 10 degrees, more specifically, about 5 degrees to about 7 degrees. For some embodiments, the electrical pulses may generate a corresponding rotation pulse of the cam shaft 70 of about 6 degrees such that 60 electrical pulses results in a corresponding 60 rotation pulses of the cam shaft 70 for a total of a 360 degree full rotation of the cam shaft 70.

This configuration provides a resolution in the rotation of the cam shaft 70 to the 6 degree value per pulse. In some cases, the controller may be configured to count the number of pulses or steps used per revolution of the cam shaft 70 and utilize an algorithm to adjust the duration of the electrical pulses in order to maintain a rotation per pulse of about 3 degrees to about 10 degrees, more specifically, about 5 degrees to about 7 degrees, and even more specifically, about 6 degrees. For some embodiments, the controller 88 may be configured to rotate the cam shaft 70 one full rotation over a time period of about 15 seconds to about 60 seconds, more specifically, about 25 seconds to about 35 seconds, during normal usage.

As discussed above, the actuator assembly 14 typically includes the pressure sensor 130 which may be disposed in fluid communication with the air volume 24 of the fluid reservoir 18. The pressure sensor 130 is also operatively coupled to the controller 88 which may be configured to monitor pressure measurements of the pressure sensor 130 from within the air volume 24 of the fluid reservoir 18. The controller 88 may also be configured to trigger an alarm indicating an occlusion in an outlet path 56 between the outlet port 52 and a subcutaneous delivery site 140 within the patient’s body 142, as shown in FIG. 3, if an unexpected pressure profile for the air volume 24 is detected by the controller 88 over a plurality of fill cycles, in some cases, if a pressure increase in the air volume 24 is detected over a plurality of consecutive dispense cycles or pumping cycles.

In other cases, such an alarm might be triggered if a lack of pressure drop in the air volume 24 is detected during a fill cycle over a plurality of pumping cycles which may be indicative of a lack of flow of therapeutic fluid 50 from the liquid volume 22 during a fill cycle. Such a subcutaneous delivery site 140 may be accessed by deploying a patient port 145 that includes a hub 146 having a flexible tubular cannula 147 extending therefrom as shown in FIG. 3. The hub 146 may be configured to establish fluid communication between an inner lumen of the flexible tubular cannula 147 and the outlet conduit 56 of the medical pump system 10. Any suitable commercially available patient port 145 may be used including an Ypsomed Orbit® Soft Infusion Set manufactured by Ypsomed AG located in Burgdorf, Switzerland.

In some cases, such an alarm may be triggered if such an unexpected pressure profile within the air chamber 24 is detected over about 2 pumping cycles to about 6 pumping cycles. In some cases an occlusion alarm may be triggered by the controller if an increase in pressure in the air chamber 24 is detected over 3 pumping cycles. In some instances, the controller 88 may also be configured to trigger an alarm indicating a pump failure if an unexpected pressure profile for the air volume 24 is detected by the controller 88 over a plurality of fill cycles. In some cases, the controller 88 may be configured to trigger a pump failure alarm if an unexpected pressure profile is detected over about 4 pumping cycles to about 6 pumping cycles. In some circumstances, the controller 88 may be configured to trigger a pump failure alarm if an unexpected pressure profile is detected over about 5 pumping cycles.

With regard to use of the pressure sensor 130, the controller 88 may also be configured to determine the amount of therapeutic fluid 50 disposed in the liquid volume 22 of the fluid reservoir 18 based on a pressure measurement taken from the pressure sensor 130. In some cases, this may be carried out by sensing a magnitude of a pressure drop within the air volume 24 during a fill cycle wherein a predetermined volume of therapeutic fluid 50 is drawn out of the liquid volume 22 and into the pump chamber 34 through the inlet port 42. Typically, a known predetermined volume of therapeutic fluid 50 is dispensed during each pumping cycle. As the therapeutic fluid 50 is withdrawn from the liquid volume 22, the pressure within the liquid reservoir 18 drops. The magnitude of the pressure drop during withdrawal of a predetermined volume of therapeutic fluid 50 may be dependent upon the amount of therapeutic fluid 50 remaining in the liquid reservoir 18 because the therapeutic fluid 50 has little to no compressibility and the air within the fluid reservoir 18 is highly compressible relative to the therapeutic fluid 50.

As such, if the liquid volume 22 is full or nearly full of therapeutic fluid 50, there will be a significantly higher drop in pressure within the liquid reservoir 18 during a fill cycle relative to a drop in pressure if the liquid volume 22 is nearly empty of therapeutic fluid 50 and the liquid reservoir 18 is filled mostly with compressible air. In some cases the actuator assembly 14 may also include a temperature sensor 131 which is disposed in operative communication with the controller 88. This allows the controller 88 to monitor ambient temperature and ambient atmospheric pressure when the vent port 100 is open as well as pressure within the liquid reservoir 22 when the vent port 100 is closed. For some embodiments, the temperature sensor may be part of the pressure sensor 130, i.e., the pressure sensor 130 also includes the temperature sensor.

For some embodiments, the actuator assembly 14 may also include a position sensor 144 which is operatively coupled to the motor 84 and/or the cam shaft 70. The position sensor, as shown in FIGS. 1 and 14A-14C, may further be operatively coupled to the controller 88 thus enabling the controller 88 to monitor the angular position of the motor 84 as well as the cam shaft 70. In some cases, the position sensor 144 may include a microswitch (not shown) having an actuator lever in contact with either a drive shaft of the motor 84 and/or the cam shaft 70. In other cases, the position sensor 144 may include a photo interrupt sensor, a hall effect sensor, a color sensor, an infrared (IR) sensor or the like.

As the reservoir cartridge assembly 12 is generally configured as limited use element of the medical pump system 10, it may be useful to include components that require frequent refreshing to be included with this assembly 12. In particular, the reservoir cartridge assembly 12 may generally include an electrical power source 148 which may be operatively coupled to the controller 88 with a conductive conduit when the reservoir cartridge assembly 12 and actuator assembly 14 are operatively coupled together. In some cases, the electrical power source 148 may include a battery. In some cases, the battery 148 may also be operatively coupled to the PCB 132 as well. Battery embodiments such as a coin cell type battery, including a CR2032 magnesium dioxide lithium battery, may be suitable for use with the medical pump system embodiments 10 in some cases.

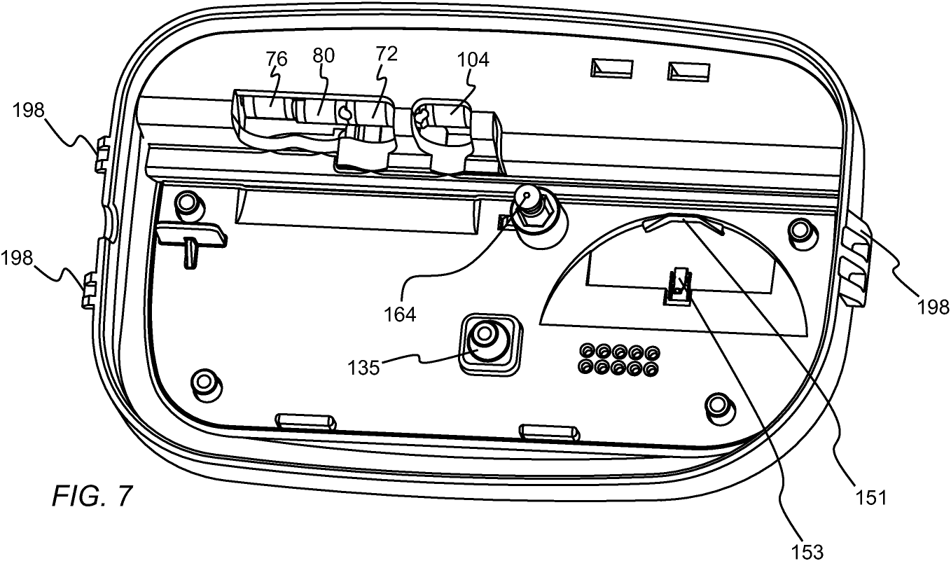

For some embodiments, the battery 148 may be secured to a portion of the reservoir cartridge assembly 12 by any suitable means and in some cases the battery 148 may be secured to a top portion of the fluid reservoir cover 95 with a double sided adhesive pad 149 that is secured on one side to the battery 148 and the opposite side to the upper side of the fluid reservoir cover 95. For some embodiments of the battery 148, a negative pole of the battery 148 may be electrically coupled to a first battery contact 151 and a positive pole of the battery may be electrically coupled to a second battery contact 153 as shown in FIG. 7, and the first battery contact 151 and second battery contact 153 may be electrically coupled to the controller 88 and/or PCB 132.

With regard to patient interface features of the medical pump system 10, for some embodiments, the actuator assembly 14 may include an indicator light 150 that is operatively coupled to the controller 88. The indicator light 150 is configured to be viewable by the end user patient and the controller 88 may be configured to communicate a variety of signals to the indicator light 150 indicative of status information regarding the medical pump system 10. In some cases, the indicator light 150 may include a tri-color light emitting diode. The actuator assembly 14 may also include an electronic sound emitter 152 that for some embodiments may include a piezo sounder disc that is audible to a patient and operatively coupled to the controller 88. Any other form of sound emitter 152 may also be so used and operatively coupled to the controller 88 including voice coil speakers and the like. In some instances, the controller 88 may be configured to communicate a variety of signals to the piezo sounder disc 152 which are configured to be converted to corresponding audible signals observable by the patient end user that are indicative of status information regarding the medical pump system 10.

The control button 134 may be disposed coextensively with an outside surface 154 of an outer shell 156 of the actuator assembly 14. The control button 134 may be accessible for manual activation by a patient and operatively coupled to the controller 88 to provide an operative interface between a patient and the controller 88 and its associated control functions and programming. The optional priming button 136 may also operatively coupled to the controller 88 to provide priming commands to the controller 88 by a patient. In some cases, the controller 88 may be configured to initiate priming of a complete fluid path from the liquid volume 22 of the fluid reservoir 18 to the outlet conduit 56 upon activation of the priming button 136. For some embodiments, the priming button 136 may be recessed into the outside surface 154 of the outer shell 156 of the actuator assembly 14 such that the priming button 136 is not easily accessible for manual activation by a patient without a priming tool (not shown) that may be configured to allow the patient to activate the priming button 136. For some embodiments, the control button 134 may be used by an end user patient to directly control a pumping method of the medical pump system 10.

In some cases, the controller 88, including the memory 91 thereof, may be configured or otherwise include instructions to have certain components of the medical pump system 10 carry out certain processes based on input from the end user patient. For example, for some embodiments, once a steady state infusion rate has been generated by the controller, the patient may use the control button 134 to initiate a bolus delivery of therapeutic fluid 50 to the subcutaneous delivery site 140. In some cases, such a bolus delivery may be instructed by a continuous press of the control button for an intermediate length duration, in some cases this might include a constant three second press of the control button 134. Thereafter, short incremental presses of the control button 134 separated by release of the control button 134 may be used to count out the volume of the bolus to be delivered. The amount of the bolus to be delivered may then be relayed back to the patient by flashes of the indicator light 150 and/or beeps generated by the piezo sounder 152. If these confirmation signals from the indicator light 150 and/or piezo sounder 152 are correct, the patient may then confirm the bolus instruction with a long continuous press of the control button 134, for example, a six second continuous press of the control button 134.

Thus, the controller 88 may be configured to deliver a desired volume of bolus delivery using only a single controller button 134 and three types of button presses, including the instantaneous incremental press followed by an immediate release of the control button 134, a continuous press and hold of intermediate length or duration, including the 3 second continuous press and a continuous press and hold of a long duration of about 6 seconds. In some instances, it may be useful for the duration of the long continuous press of the control button 134 to be twice or more that of the intermediate continuous press in order for the patient to be able to easily distinguish between these two types of button presses.

In some cases, the controller 88 and associated components thereof may be configured such that an in progress bolus delivery may be canceled by a long duration continuous press of the control button 134. In addition, the controller 88 and associated components thereof may be configured such that an in progress controlled infusion rate, such as a basal rate, delivery may be canceled by a long duration continuous press of the control button 134. In addition, during normal operation of some medical pump system embodiments 10, a status check may be initiated by the patient by a single short press of the control button 134 followed by immediate release. The short press and release of the control button 134 may also be used to acknowledge any alerts being transmitted by the controller 88 through the piezo sounder 152 and/or indicator light 150.

For some embodiments, the actuator assembly 14 may include the optional transmission 160 which is operatively coupled between the cam assembly 68 and the motor 84. For some motor embodiments, such a transmission 160 may not be necessary. However, for some motor embodiments 84, it may be useful to include such a transmission 160 in order to better control rotation of the cam shaft 70 with a suitable gear reduction ratio. In some cases, the transmission 160 may include a planetary gear box or the like.

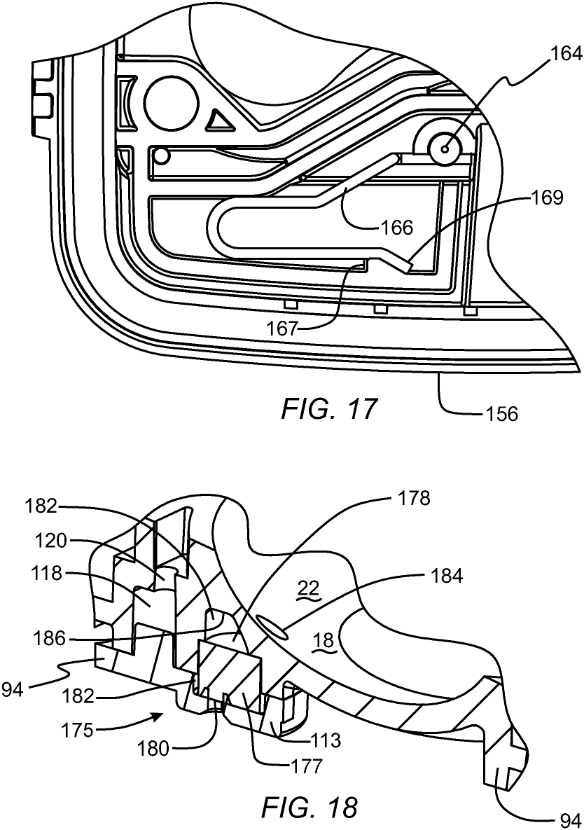

Some embodiments of the actuator assembly 14 may include the actuator chassis 162 and the cam assembly 68, motor 84 and transmission 160 may be disposed on the actuator chassis 162. The actuator assembly 14 may also include a latch post 164 secured to and extending from a bottom surface of the actuator chassis as shown in FIG. 7. The reservoir cartridge assembly 12 may in turn include a latch spring 166 that is disposed on the reservoir base 94 of the reservoir cartridge assembly 12 as shown in FIGS. 8 and 17. The latch spring 166 may be configured to latch onto a groove or slot 168 the latch post 164, as shown in FIG. 5, to prevent separation of the actuator assembly 14 from the reservoir cartridge assembly 12 after they have been operatively coupled together.

In some instances, the latch spring 166 may be configured to be permanently disabled after being disengaged from a latch post 164 to prevent multiple uses of the latch spring 166. As such, the latch mechanism 16 may include a disabling feature 167, shown in FIG. 17, which the latch spring 166 is spring biased against and which includes a ramped end and a flat end. When the latch spring 166 is disengaged by translating the latch spring 166 to the left with the priming tool or any other suitable instrument, the biased end 169 of the latch spring 166 slides up and over the ramp end and elastically snaps into engagement behind the flat end with the latch spring in a position where it no longer is able to engage the groove 168 of the latch post 164, thus disabling the latch mechanism embodiment 16. The disabling feature 167 shown in FIG. 17 is an exemplary embodiment and any other suitably configured mechanism may also be used.

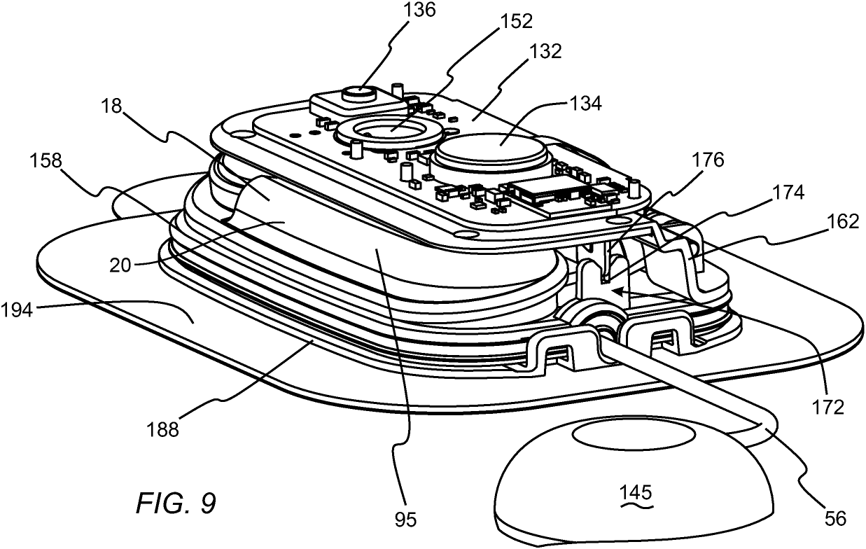

With regard to coupling the reservoir cartridge assembly 12 to the actuator assembly 14, in some instances, the actuator chassis 162 and the reservoir base 94 may include a mutual alignment feature 172 as shown in FIG. 9 which may be configured to be operatively and releasably coupled between the actuator chassis 162 and the reservoir base 94 that prevents relative lateral displacement between the actuator chassis 162 and the reservoir base 94. In some cases, the alignment feature 172 may include an alignment slot 174 secured in fixed relation to reservoir base 94 and an alignment rib 176 which is secured in fixed relation to the actuator chassis 162 with the alignment rib 176 being configured to fit tightly within the alignment slot 174. The arrangement could also be reversed with the alignment slot 174 disposed on the actuator chassis 162 and the alignment rib 176 disposed on the reservoir base 94. Any other suitable type of alignment feature 172 may be used as well in order to limit or prevent any relative lateral displacement between the actuator chassis 162 and reservoir base 94 during rotation of the cam assembly 68 against the pushrods of the pump assembly.

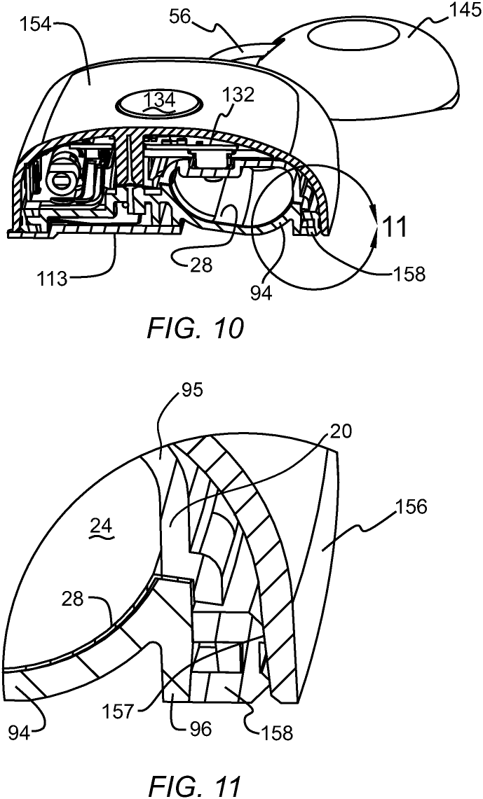

For some embodiments, the actuator assembly 14 may also include the outer shell 156 which may be a smooth continuous layer of rigid material which is disposed over and protects all of the components of the actuator assembly 14 from environmental elements, including moisture. As such, in some cases, an outer shell seal 158 may be disposed about an outer perimeter 96 of the reservoir base 94, as shown in FIGS. 10 and 11, and be sized and configured to seal to an inside surface 157 of the outer shell 156 when the actuator assembly 14 is coupled to the reservoir cartridge assembly 12. In some cases, the outer shell seal 156 may include a flexible elastomer with a double lip transverse cross section as shown in FIGS. 10 and 11. The contact parameters between the inside surface 157 of the outer shell 156 and the outer shell seal 158 may be configured, in some instances, to provide a level IP24 rated seal therebetween.

The reservoir cartridge assembly 12 may include a fill port 175 to facilitate manual filling of the liquid volume 22 of the fluid reservoir 18 as shown in FIG. 18 once the reservoir cartridge assembly 12 has been coupled to the actuator assembly 14. For some embodiments, the fill port 175 may include a fill septum 177 which has an inner surface 178, an outer surface 180 and which is disposed within a fill septum cavity 181. The fill septum cavity 181 may include an open portion 182 disposed adjacent the inner surface 178 of the fill septum 176. The open portion 182 may also be disposed in fluid communication with a fill passage 184. The fill passage 184 may in turn be disposed in fluid communication between the fill septum cavity 181 and the liquid volume 22 of the fluid reservoir 18.

A patient may generally use the fill port 175 by filling an appropriately configured syringe with a desired therapeutic fluid 50 and inserting the hypodermic needle of the syringe (not shown) through the fill septum 176 and into the open portion 182 of the fill septum cavity 181 with a distal port of the hypodermic needle in fluid communication with the open portion 182 of the fill septum cavity 181. The syringe may then be emptied into the open portion 182 of the fill septum cavity 181 with the therapeutic fluid 50 so delivered flowing through the fill passage 184 into the liquid volume 22. In some instances, the open portion 182 may include a needle stop surface 186 formed by an inner surface of the fill septum cavity 181 disposed opposite the inner surface 178 of the fill septum 176. The needle stop surface 186 may be configured and positioned to prevent unwanted penetration of the hypodermic needle into fragile components of the medical pump system 10. For some embodiment, the fill septum 176 may include an elastic polymer such as polyurethane, silicone or the like.

Once the liquid volume 22 of the fluid reservoir 18 of the medical pump system 10 has been filled, the patient will generally attach the medical pump system 10 to a desired position on their body 142. Some embodiments of the medical pump system 10 discussed above may include an optional mount bracket 188 that may be used to releasably secure the coupled actuator assembly 14 and reservoir cartridge assembly 12 to an outer surface of the patient’s skin 143 in a desired location. Referring to FIGS. 2-4, the mount bracket 188 may include a bracket body 190 having an adhesive layer 192 disposed on a bottom surface of an adhesive pad 194 that is secured to the bracket body 190. Typically, the adhesive surface of the adhesive layer 192 will be covered by a peel off layer that maintains the integrity of the adhesive surface until ready for use. The bracket body 190 may have an outer contour that generally matches an outer contour of the perimeter of the outer shell 156 of the actuator assembly 14. The bracket body 190 also includes a plurality of mount receptacles 196 that are configured to receive corresponding mount tabs 198 disposed on the outer perimeter of the outer shell 156 (see also FIG. 7).

One of the mount receptacles 196 may include a flexible bail 200 that may have a resilient flexibility that allows a mating mount tab 198 of the outer shell 156 to be snapped into place with an opening of the flexible bail 200 mechanically capturing the mating mount tab 198. Once the end user patient is ready to remove the actuator assembly 14 from the mount bracket 188, the flexible bail 200 may be elastically flexed outwardly away from the outer shell 156 so as to disengage the flexible bail 200 from the mating mount tab 198 disposed therein thereby releasing the actuator assembly 14 and reservoir cartridge assembly 12 coupled thereto from the mount bracket 188. The flexible bail 200 is elastically deformed and thus reusable if desired. For some mount bracket embodiments 188, the adhesive pad 194 may have a length of about 2.5 inches to about 3 inches, a width of about 2 inches to about 2.5 inches and a thickness of about 0.02 inches to about 0.05 inches. The adhesive surface 192 may include any adhesive suitable for skin contact including acrylate type adhesives or the like.

The coupled actuator assembly 14 and reservoir cartridge assembly 12 may also be attached to the patient’s body 142 in other ways. For example, in some cases, a flexible polymer layer 141 separate from the medical pump system 10 may be used. As such, for some kit embodiments that include medical pump system embodiments 10, such a kit may also include an optional flexible polymer layer or patch 141 shown schematically in FIG. 3 which is sized to fit over the actuator assembly 14 and assembled medical pump system 10 as a whole. The flexible polymer patch 141 may include an adhesive backed perimeter portion which is configured to be releasably secured to a patient’s skin 143. For some embodiments, the flexible polymer patch 141 may have at least one vent hole disposed therethrough.

For such an application, the flexible polymer patch 141 may be disposed over the medical pump system 10 against the patient’s skin 143 and the adhesive backed perimeter portion thereof secured to the patient’s skin 143 creating a pouch against the patient’s skin 143 in which the medical pump system 10 may be disposed. Once the medical pump system 10 needs to be removed or otherwise accessed, the flexible patch would be removed from the patient’s skin 143 and a new flexible patch 141 used to reattach the medical pump system 10 to the patient. Typically, the flexible polymer patch 141 would be made of a clear flexible material that would permit operation of the control button 134 and priming button 136 and would also transmit any audio or visual signals therethrough.

In some cases, a patient may initiate use of medical pump system embodiments 10 discussed herein by coupling the reservoir cartridge assembly 12 to the actuator assembly 14 to form the medical pump system 10. In some cases, the coupling of the reservoir cartridge assembly 12 to the actuator assembly 14 may be detected by the controller 88 by the controller 88 which detects electrical power being supplied to the controller 88. The controller 88 may then initiate a power-on-self-test once the controller 88 has detected electrical power being supplied to the controller 88. In addition, a time point zero may be stored into the memory 91 of the controller 88 and a power source voltage check initiated by the controller 88. In some instances, at this stage, the controller 88 may be configured to perform one complete rotation of the cam assembly 68 or cam shaft 70 of the actuator assembly 14 with the cam shaft 70 coming to a stop in an angular position wherein the inlet port 42 of the pump chamber assembly 32 of the reservoir cartridge assembly 12 is closed and the vent port 100 of the pump chamber assembly 32 of the reservoir cartridge assembly 12 is open to the ambient atmosphere.

Thereafter, the liquid volume 22 of a fluid reservoir 18 of the reservoir cartridge assembly 12 may be manually filled with the therapeutic fluid 50 while venting air from the air volume 24 disposed adjacent the liquid volume 22. The liquid volume 22 may be filled through the fill port 175 with a syringe or other suitable source of desired therapeutic fluid 50. As discussed above, the therapeutic fluid 50 injected into the fill port 175 flows through the fill passage 184 and into the liquid volume 22 bounded by a fluid cavity molded into the reservoir base 94 and the flexible membrane 28 sealed thereto.

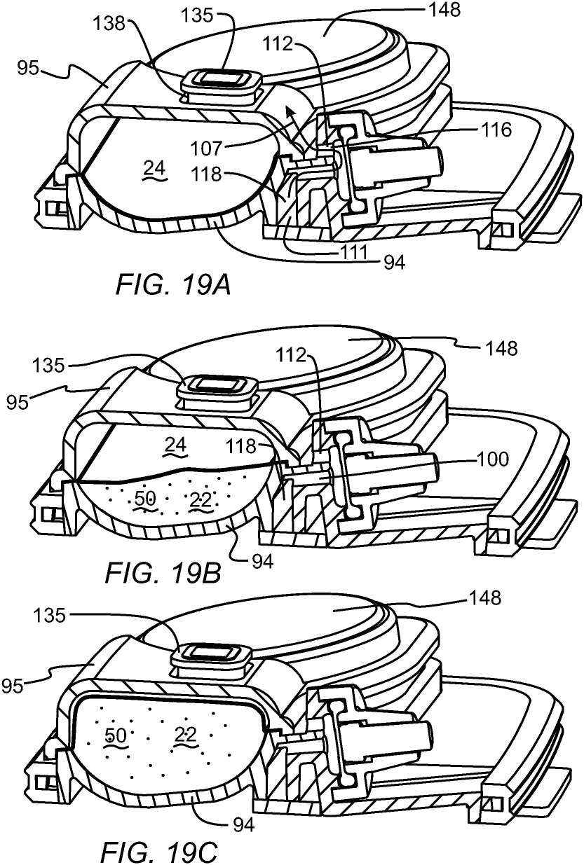

In some cases, the flexible membrane 28 may be pre-molded or otherwise form fitted to the fluid cavity molded into the reservoir base 94 to reduce or eliminate any air pockets in the liquid volume 22 when the liquid volume is empty as shown in FIG. 19A. As therapeutic fluid 50 flows into the liquid volume space, the flexible membrane 28 begins to distend and separate from the fluid cavity surface of the reservoir base 94 as the incoming therapeutic fluid begins to displace the flexible membrane as illustrated by the partially filled liquid volume shown in FIG. 19B. The liquid volume 22 may continue to be filled through the fill port 174 until the liquid volume 22 of the fluid reservoir 18 is completely filled with the flexible membrane 28 being pressed up against or adjacent to the fluid reservoir cover 95 as shown in FIG. 19C. For some embodiments, the fluid reservoir cover 95 as well as the reservoir base 94 may be formed from clear polymer materials in order for a patient to be able to visualize the fill level within the liquid volume 22 of the fluid reservoir 18.

The outlet conduit 56 of the pump chamber assembly 32 may be primed by activating the priming button 136 of the actuator assembly 14 and the medical pump system 10 releasably secured to the patient. In some instances, releasably securing the medical pump system 10 to the patient may include removing a backing of the adhesive pad 194 of the mount bracket 188 of the medical pump system embodiment 10, applying the adhesive surface 192 of the adhesive pad 194 to the patient’s skin 143 in a suitable location and releasably securing the actuator assembly 14 and reservoir cartridge assembly 12 of the medical pump system 10 to the mount bracket 188. In other cases, releasably securing the medical pump system 10 to the patient may include disposing a flexible polymer layer over the medical pump system 10 and sealing the perimeter of the flexible polymer layer to the patient’s skin 143 around the medical pump system 10 as discussed above. Once the medical pump system 10 is so secured, the outlet conduit 56 of the pump chamber assembly 32 may be disposed in fluid communication with a subcutaneous delivery site 140 within the patient’s body 142. A controlled rate of infusion, such as a basal rate, of the therapeutic fluid 50 may then be delivered to the subcutaneous delivery site 140 of the patient by performing sequential pumping cycles of the medical pump system 10 carried out according to a predetermined delivery protocol.

In some instances, performing such a pumping cycle may include performing a fill cycle of the cam assembly 68 followed by performing a dispense cycle of the cam assembly 68 by rotation of the cam shaft 70. In some embodiments, performing a fill cycle by rotation of the cam shaft 70 may include disposing the inlet cam lobe 72 in a retracted position with an inlet port 42 of the pump chamber assembly 32 in an open position as shown in FIG. 14A. The fill cycle may also include disposing the outlet cam lobe 76 in an extended position with an outlet port 52 of the pump chamber assembly 32 in a closed position and disposing the displacement cam lobe 80 in an extended position.

Thereafter, with the inlet port 42 open and the outlet port 52 closed, the displacement cam lobe 80 may be retracted as indicated by the arrow 202 in FIG. 14A so as to expand the displacement chamber 62 and draw therapeutic fluid 50 through the inlet port 42 and into the expanding displacement chamber 62 as indicated by arrow 204 in FIG. 14A. Such drawing in of the therapeutic fluid continues until the displacement cam lobe 80 is fully retracted and the displacement chamber 62 is full of therapeutic fluid 50 as shown in FIG. 14B. During this fill cycle, the controller 88 may be configured to determine a fill level of the liquid volume 22 of the fluid reservoir 18 by monitoring a pressure drop within the air volume 24 of the fluid reservoir 18 with the controller 88 during the fill cycle. In addition, embodiments of the fill cycle may terminate by extending the inlet cam lobe 72 until the inlet port 42 is closed while maintaining the displacement cam lobe 80 in a retracted position and with the outlet port 52 closed as shown in FIG. 14B.

In some cases, performing the dispense cycle by rotation of the cam shaft 70 may include retracting the outlet cam lobe 76 and opening the outlet port 52 while the displacement chamber 62 is full of therapeutic fluid 50 and while the inlet port 42 is closed. The dispense cycle may also include extending the displacement cam lobe 80 as indicated by arrow 205 in FIG. 14B and reducing a volume of the displacement chamber 62 and dispensing the therapeutic fluid 50 disposed therein out of the outlet port 52 as shown by arrow 206 while the inlet port 42 is closed and the outlet port 52 is open.

The dispense cycle may also include opening the vent port 100 of the pump chamber assembly 32 and venting the air volume 24 of the fluid reservoir 18 to an ambient atmosphere as indicated by arrow 107 during the dispense cycle. The dispense cycle, in some instances, may also include maintaining the vent port 100 in an open state and venting the air volume 24 of the fluid reservoir 18 to the ambient atmosphere while waiting for a subsequent dispense cycle so as to monitor ambient pressure and detect any unexpected ambient pressure profiles. During both the fill cycle and dispense cycle, rotation of the cam shaft 70 may be monitored by the controller 88 using the position sensor 144.

For some embodiments and delivery of certain therapeutic fluids 50, delivering a controlled rate of infusion, such as a basal rate, of the therapeutic fluid 50 may include delivering about 5 microliters to about 15 microliters of therapeutic fluid 50 per hour to the subcutaneous delivery site 140. For some embodiments, the fluid reservoir 18 may have a volume capacity of about 2 ml to about 5 ml, more specifically, about 2.8 ml to about 3.2 ml. Once the therapeutic fluid 18 disposed within the liquid volume 22 of the fluid reservoir 18 is used up, or close to being used up, an alarm signal may be triggered by the controller 88. In addition, with regard to a time limit for the reservoir cartridge assembly 12 programmed into the controller memory 91, expiry of the reservoir cartridge assembly 12 may indicated and triggered after about 60 hours of use to about 100 hours of use by activating an alarm signal with the controller 88. Such an alarm signal will be observable by the patient either visually or audibly.

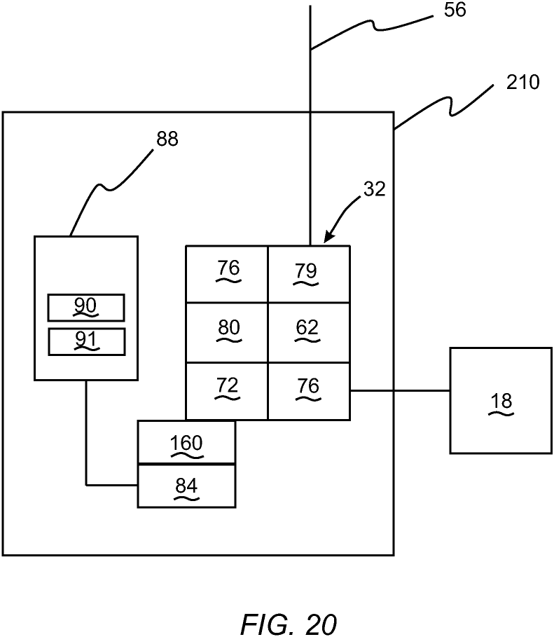

In some cases, certain components or subassemblies of the medical pump system embodiments 10 discussed herein may be useful separately or as part of another pump system embodiment. Referring to FIG. 20, a schematic representation of a pump assembly embodiment 210 for medical use is shown that includes components of the medical pump system embodiments 10 discussed above and that may perform the same functions in the same manner as discussed above. The pump assembly embodiment 210 may include the pump chamber assembly 32 including the pump chamber 34 having the interior volume 36 which is at least partially bounded by the pump housing 38. The pump chamber assembly 32 may also include the inlet port 42 which is in fluid communication with the interior volume 36. The pump chamber assembly 32 may also include the resilient inlet membrane 44 which is disposed adjacent the inlet port 42, which is spaced from the inlet port 42 when in a relaxed state, and which is sufficiently distendable towards the inlet port 42 to seal the inlet port 42 when in the compressed state.

The pump chamber assembly 32 may also include the outlet port 52 in fluid communication with the interior volume 36 and the resilient outlet membrane 54 which is disposed adjacent the outlet port 52, which is spaced from the outlet port 52 when in a relaxed state, and which is sufficiently distendable towards the outlet port 52 to seal the outlet port 52 in the compressed state. The displacement chamber 62 may also be disposed within the interior volume 36 and the resilient displacement membrane 64 is disposed adjacent the displacement chamber 62, which forms at least a portion of a boundary of the displacement chamber 62, which is sufficiently inwardly distendable from a relaxed state to reduce the volume of the displacement chamber 62 when in the compressed state. The resilient displacement membrane 64 may also be sufficiently resilient to increase the volume of the displacement chamber 62 when released from the compressed state.

The pump assembly 210 may also include the actuator assembly embodiment 14 having the cam assembly 68 with the cam shaft 70 that includes the inlet cam lobe 72 which is operatively coupled to the resilient inlet membrane 44, the outlet cam lobe 76 which is operatively coupled to the resilient outlet membrane 54, and the displacement cam lobe 80 which is operatively coupled to the resilient displacement membrane 64. The actuator assembly 14 may also include the motor 84 operatively coupled to the cam assembly 68 and the controller 88 operatively coupled to the motor 84. The power source 148 such as a battery may also be operatively coupled to the controller 88. All of the components of the pump assembly embodiment 210 shown in FIG. 20 may function in the same manner as discussed herein with regard to other pump system embodiments 10. In addition, for some embodiments, the inlet cam lobe 72 may be operatively coupled to the inlet valve assembly 75, the outlet cam lobe 76 may be operatively coupled to the outlet valve assembly 79 as well as the displacement cam lobe 80 being operatively coupled to the displacement chamber and its associated components including the displacement pushrod 82.

Proper compliance with regard to use of medical devices may typically be achieved by providing instructions for use (IFU) to a patient or treating physician. Such IFUs often include an overwhelming amount of information and warnings forewarning against negative consequences. Due to the patient’s challenge in becoming acceptably knowledgeable, educators and physicians are often required to provide a personal level of education and training support for treatments being administered. This practice may, in some circumstances, create a financial burden to the patient end user and also result in inconsistent success rates with regard to compliance due to the lack of consistency in training methods.

Devices that require user input in order to complete a specific function related to the therapy being administered may have reduced effectiveness if used the patient end user does not fully understand how to complete the specific function correctly. For instance, use of an ambulatory insulin infusion pump intended for use by an end user patient with diabetes that does not have any professional medical training typically requires the end user to select a quantity of insulin to be infused based on the current blood sugar level or meal they plan to consume which may be fairly complicated in some instances. As such, it may be desirable to provide a device and/or method for such an end user to engage in hands on training of the particular device they will be using without the risk of a potentially costly mistake in order to gain familiarity and confidence prior to actual therapeutic use.

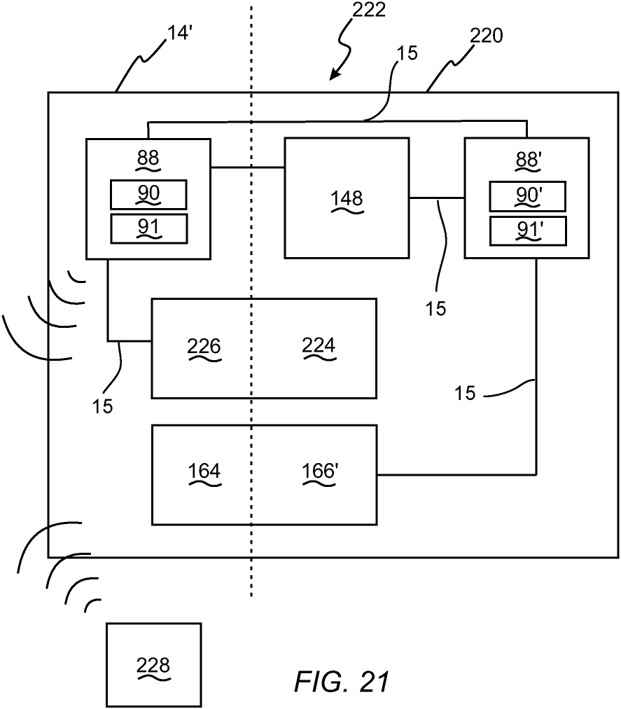

As such, certain medical devices, such as medical pump systems 10 or components thereof may be supplied with an attachable training module or cartridge 220 that may be combined to form a medical pump training system 222 as schematically illustrated in FIG. 21. Such a medical pump training system 222 may enable the end user patient to simulate the use of the medical device in a safe environment without the risk of wasted medical materials or equipment. The schematic overview of the embodiment of the medical pump training system 222 shown in FIG. 21 includes a dashed line which indicates a coupling interface between the actuator assembly 14ʹ and the training cartridge 220 of the medical pump training system 222, and the various components thereof. The coupling interface represents the physical point of separation between the respective components of each of the actuator assembly embodiment 14ʹ and training cartridge 220 that interface with each other when the actuator assembly 14ʹ and training cartridge 220 are coupled together.

The interconnecting lines between various schematic components of FIG. 21 may be conducting conduits 15 and may include any type of suitable conduit that may be useful for operatively interconnecting the components such as information conducting conduits, power conducting conduits or the like including conductive wires, optical fibers, wireless connectivity etc. In general, the actuator assembly embodiment 14ʹ may include any or all of the components of the actuator assembly 14 discussed above. The actuator assembly embodiment 14ʹ in FIG. 21 is shown without any of the cam lobes 72, 76, 80 or 104 for clarity of illustration and because these components of the actuator assembly may not typically have any counterpart components in the training cartridge 220 to interface with. Likewise, the motor 84 and transmission 160 of the actuator assembly embodiment 14ʹ are not shown for purposes of clarity of illustration.

In the case of medical pump embodiments, such as insulin pump embodiments, such a training cartridge 220 may be installed in place of the reservoir cartridge assembly 12 (reservoir) that includes or could include therapeutic materials, thereby eliminating the potential risk of over infusion of a drug, such as insulin, and allow the end user to be accustomed to the associated device inputs, outputs, interface protocols etc. For some embodiments, such a training cartridge 220 may also allow the therapeutic device, such as the actuator assembly embodiments 14' to receive updates to software stored in the memory 91, 91ʹ of the respective controllers 88, 88ʹ, or customizations in firmware, ensuring the device 14ʹ is in a safe mode that minimizes the risk to the user. This type of arrangement may be particularly useful for medical pump system embodiments 10 that include a durable element and a low use or disposable element, such as the actuator assembly embodiments 14 and reservoir cartridge assembly embodiments 12 discussed above.

With regard to medical pump systems 10 as discussed herein, as well as others, suitable training cartridge embodiments 220 may include certain elements that are typically included in a therapeutic cartridge in order to enable functioning of the system as a whole. In particular, some embodiments of the training cartridge 220 for use with the actuator assembly embodiments 14ʹ discussed herein may be used in place of the reservoir cartridge assembly embodiments 12 discussed herein. The training cartridge 220 may include an element that provides power to a functioning actuator assembly 14ʹ and may be configured to latch onto the actuator assembly 14ʹ in a similar fashion to that of the reservoir cartridge assemblies 12 (disposable) enabling the end user to exercise the physical elements of the medical pump system embodiments 10 and become familiar/comfortable with them.

In some cases, it may be desirable for the latch spring 166ʹ of the training cartridge 220, as shown in FIG. 21, to be reusable so that the training cartridge 220 may be used multiple times for multiple training sessions. As such, the training cartridge 220 may be configured to be reused and reattached while the therapeutic reservoir cartridge assembly embodiments 12 discussed herein are generally intended for single use and have a single use release latch 166. As such, the latch spring 166ʹ may be configured to not include the disabling feature 167 that is part of the latch mechanism 16 discussed above or may include any other suitable features or lack thereof that configures the latch mechanism of the training cartridge 220 to be reusable. In addition, for some embodiments of the training cartridge 220, the portion of the latch mechanism housed in the training cartridge 220, may include an electro-mechanical structure that is configured to be releasably secured to the latch post 164, and controllable by the controller 88ʹ in order to be released from the latch post 164.

The training cartridge embodiments 220 may also have a means for the mating actuator assembly 14ʹ to differentiate the training cartridge 220 from the therapeutic reservoir cartridge assembly embodiments 12 so the actuator assembly 14ʹ configures itself to operate in a training mode. In some cases, when the actuator assembly 14ʹ is coupled to the training cartridge 220, the controller 88 may be disposed in operative communication with the controller 88ʹ, and the controller 88ʹ may be configured to identify the training cartridge 220 by communicating an identification signal to the controller 88. In addition, other techniques for establishing such differentiation may include providing a certain identifying feature or features 224 on the training cartridge 220 that may be read or otherwise interpreted by an optional reader 226 that may be operatively coupled to the controller 88. In some cases, the training cartridge 220 may also include an optional training module controller 88ʹ that may be operatively coupled to power source 148 as well as the controller 88 of the actuator assembly 14ʹ. Such a training cartridge controller 88ʹ may be configured to communicate with the controller 88 of the actuator assembly 14ʹ and provide identifying information, training programs and the like.

Regarding examples of identifying features 224, some embodiments of the training cartridge 220 may include an identifying feature 224 including NFC tag, a 2D barcode on training cartridge with read camera 226 on actuator assembly 14ʹ, a resistive label disposed on the training cartridge 220 with corresponding contacts on actuator assembly 14ʹ, a mechanical feature disposed on the training cartridge 220 that actuates a switch 226 on the actuator assembly 14ʹ or a magnet on the training cartridge and hall effect sensor 226 on actuator assembly 14ʹ.

In use, for some embodiments, a training mode may provide specific user functions and disengage critical alarms caused from failure detections on the actuator assembly 14ʹ (e.g., occlusion detection, low insulin, pump malfunction). The actuator assembly 14ʹ may, in some cases, wirelessly connect (BLE) to a remote mobile device 228, such as a smart phone or the like with supporting application as shown in FIG. 21. In some cases, a training mode of the actuator assembly 14ʹ may unlock a training section of the phone application. For some embodiments, the remote mobile device 228 may be configured to provide real time guidance and feedback to a user of the device based on a status of a condition being monitored by the controller 88, or in some cases, controller 88ʹ. In some instances, the remote mobile device 228 may be configured to communicate status data regarding conditions being monitored by the controller 88, or in some cases controller 88ʹ, to a cloud data management system through a wireless connection between the remote mobile device 228 and the cloud data management system (not shown).

Such a training application for a remote mobile device 228 or the like may include providing tutorials, videos, and/or a dashboard providing additional information on actuator assembly features. The training mode of the actuator assembly 14ʹ may also include the ability to switch from training mode to operation mode based on pump feedback driven by the training cartridge 220 being installed. A connection to a data portal or the like in order to upload status and provide real time tracking and support of the actuator assembly 14ʹ or any other component of the medical pump system 10 from an administrative account. In some cases, support analysis of user training sessions may be communicated to the controller 88 in order to ensure that the medical pump system 10 or components thereof are being utilized in a safe and useful manner.

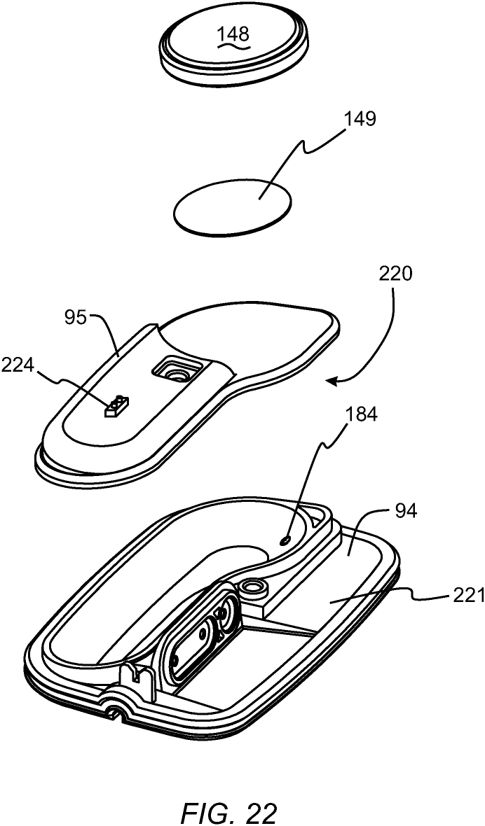

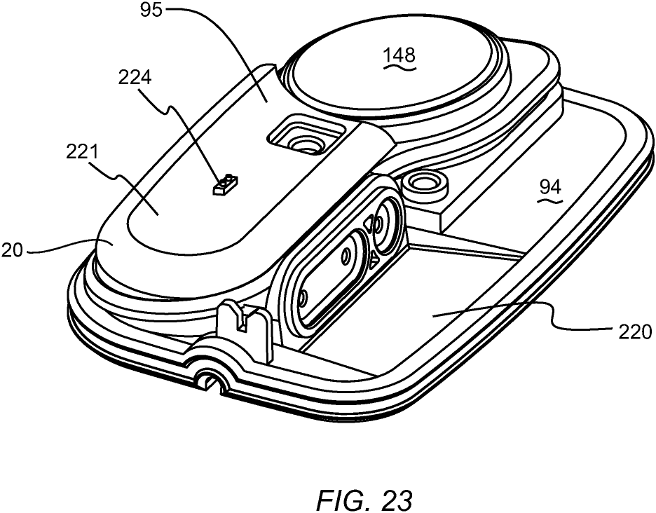

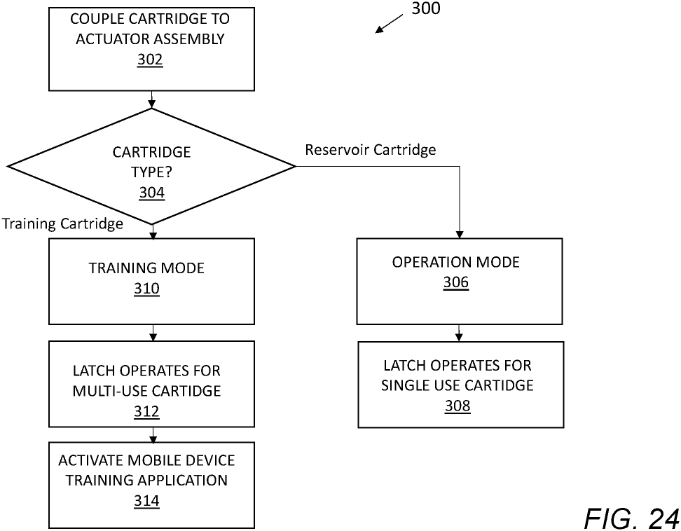

Referring to FIGS. 21-23, the medical pump training system 222 is shown that may include the actuator assembly 14ʹ having the actuator chassis 162 and the controller 88 secured to the actuator chassis 162. The medical pump training system 222 may also include the training cartridge 220 having a cartridge housing 221 which is configured to releasably couple to the actuator assembly 14ʹ and which includes the identifying feature 224 that is configured to be operatively coupled to the controller 88 of the actuator assembly 14ʹ and provide information to the controller 88 identifying the training cartridge 220 as a non-therapeutic cartridge. In some cases, the identifying feature 224 may be operatively coupled to the controller 88 by means of the reader 226 which may be operatively coupled to both the controller 88 and the identifying feature 224.

In some cases, a memory 91 of the controller 88 may include instructions, which may include machine readable instructions, to initiate a training program for a patient once the controller 88 identifies the training cartridge 220 by receiving the information from the identifying feature 224. As discussed above, suitable examples of identifying feature embodiments 224 may include an NFC tag. The identifying feature 224 may also include a 2D barcode disposed on the training cartridge 220 and the actuator assembly 14ʹ may further include a read camera 226 which is configured to read the 2D barcode. In some instances, the identifying feature 224 may be a resistive label and in such cases the actuator assembly 14ʹ may have corresponding electrical contacts that are configured to operatively couple to the resistive label such that the controller 88 will be configured to determine the resistance of the resistive label and identify the training cartridge 220.

For some embodiments, the identifying feature 224 may include a mechanical feature and the actuator assembly 14 further comprises a switch and the mechanical feature is configured to actuate the switch on the actuator assembly 14ʹ. In some cases, the identifying feature 224 may include a magnet disposed on the training cartridge 220 and the actuator assembly 14ʹ may have a corresponding a hall effect sensor which is configured to be operatively coupled to the magnet to enable the controller 88 which is operatively coupled to the hall effect sensor to identify the training cartridge 220. As noted above, it may be desirable for the training cartridge 220 to be reusable. As such, in some instances, the cartridge housing 221 may further include a reusable latch spring 166ʹ that is configured to releasably couple to a latch post 164 which is secured in fixed relation to the actuator assembly 14ʹ.

Some embodiments of a training cartridge 220 for a medical pump system 10 may include the cartridge housing 221 that is configured to couple to an actuator assembly 14ʹ of the medical pump system 10 and the identifying feature 224 disposed on the cartridge housing 221 that is configured to be operatively coupled to a controller 88 of the actuator assembly 14ʹ and provide information to the controller 88 identifying the training cartridge 220. For some such training cartridge embodiments 220, the cartridge housing 221 may further include the reusable latch spring 166ʹ that is configured to releasably couple to the latch post 164 of the actuator assembly 14ʹ.

Referring to FIG. 24, a method 300 for operating embodiments of the medical pump system 10 is shown. When describing the method 300, reference is made to elements of the actuator assembly 14ʹ described herein. At step 302, a cartridge is coupled to the actuator assembly 14, for example. In some cases, the cartridge being so coupled may be either a reservoir cartridge assembly embodiment 12 or a training cartridge 220. In doing so, power (voltage, current, etc.), data and control signals, and/ or therapeutic fluid 50 or other matter are capable of exchange between the actuator assembly 14ʹ and the cartridge. At decision diamond 304, a type of cartridge is determined. In some embodiments, the type of cartridge is determined by hardware, for example, one or more interfaces, input/output devices, or the like of the actuator assembly 14ʹ. In other embodiments, the type of cartridge is determined by software, for example, stored in a computer memory at the cartridge, the actuator assembly 14ʹ, or an external computer location, such as a remote computer, a cloud computing environment, and so on that communicates with the actuator assembly 14ʹ and/or cartridge to identify the type of cartridge. In other embodiments, the cartridge is identified by a combination of hardware and software.

If at decision diamond 304, a reservoir cartridge assembly 12 is identified as coupled to the actuator assembly 14ʹ, then the method 300 proceeds to step 306 where the medical pump system 10 is in an operation mode, for example, capable of use for delivery of a therapeutic fluid 50 to a patient end user. At step 308, the latch mechanism 16 as part of a coupling mechanism between the reservoir cartridge assembly 12 and the actuator assembly 14ʹ, for example, the latch spring 166 shown in FIGS. 1 and 8, operates to permit a single use of the reservoir cartridge assembly 12 and prohibits a subsequent coupling of the same reservoir cartridge assembly 12 to the actuator assembly 14ʹ. If at decision diamond 304, a training cartridge 220 is identified as coupled to the actuator assembly 14ʹ, then the method 300 proceeds to step 310, where the medical pump is in a training mode, for example, capable of training a user. At step 312, the latch 16 as part of a coupling mechanism between the training cartridge 220 and the actuator assembly 14, for example, the latch spring 166ʹ shown in FIG. 21, operates to permit multiple uses of the training cartridge 220.

At step 314, the medical pump system 10 can connect wirelessly, for example, using BLE or other wireless interface, to a remote mobile device, such as a smartphone 228 or other computer having display or other input/output devices for permitting a trainee or other user to communicate with the medical pump system 10. When the training mode is determined at step 310, at step 314 the remote mobile device 228 may include an application that is activated, or unlocked, for example, in response to a receipt of a signal comprising data from the controller 88ʹ of the training module 220. Returning to decision diamond 304, although a decision is made here whether the medical pump system 10 is in an operation mode or a training mode, some embodiments may include the ability to switch from the training mode to the operation mode based on pump feedback or other data driven by the training module 220.

In some cases, it may be useful to have the option to pump more than one fluid with the use of a single pump mechanism. Some embodiments of medical pump systems with multiple separate fluid reservoirs and including an appropriate reservoir selector assembly may be useful to eliminate a need to integrate multiple pump chambers or pump mechanisms when multiple fluids are to be delivered. The use of multiple pump mechanisms may be a burden to the user because of size and cost of multiple pumping mechanisms. Integrating the hardware into a single pump mechanism may also reduce or eliminate a cybersecurity risk of independent systems synchronized wirelessly. In such cases, pumping mechanisms such as the pump chamber assembly embodiment 32 discussed above may be used in conjunction with a multiple fluid reservoir version of the reservoir cartridge assembly 12 that has multiple separate liquid or fluid volumes that may contain the same or different fluids including liquids and gases. A variety of such medical pump system embodiments are contemplated that may include varying arrangements of the fluid reservoirs 18, liquid or fluid volumes 22, air volumes 24, associated pressure sensors 130, temperature sensors 131, vent conduits 111 and the like for the controlled delivery of a therapeutic fluid 50 or any other suitable fluid including suitable liquids and gases.

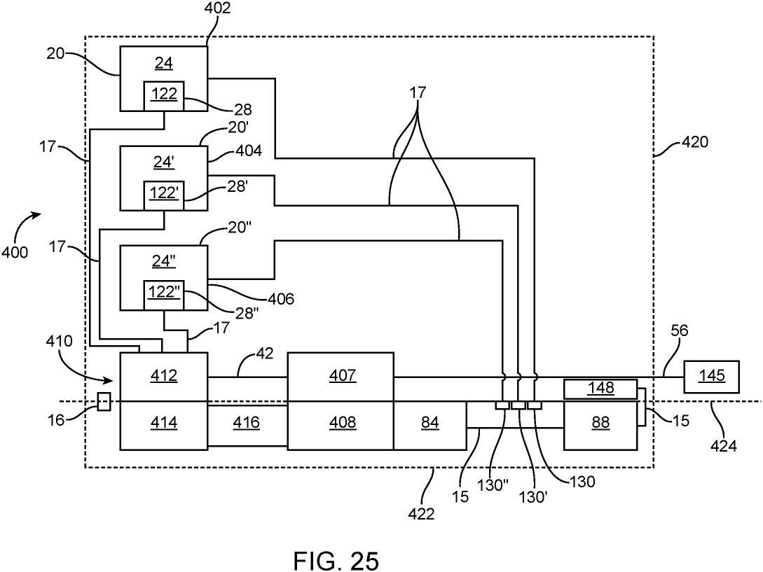

FIG. 25 shows a schematic representation of a medical pump system embodiment 400 that includes a first fluid reservoir 402, a second fluid reservoir 404 and a third fluid reservoir 406. Although the medical pump system embodiment 400 is shown with three fluid reservoirs 402, 404, 406, any suitable number of multiple fluid reservoirs may be used including 2, 3, 4, 5 or more fluid reservoirs in some cases. Each of the fluid reservoirs 402, 404, 406 may include separate respective fluid volumes 122, 122ʹ, 122ʺ separated from respective air volumes 24, 24ʹ, 24ʺ by the respective flexible membranes 28, 28ʹ, 28ʺ and surrounded by respective rigid outer containers 20, 20ʹ, 20ʺ. Regarding fluid volumes 122, 122ʹ, 122ʺ, any or all of these fluid or liquid volumes may contain liquids or suitable gases such as air, inert gases such as argon, helium, or the like. In the case of air, it may be drawn in rather than stored. The air may, in some cases, be drawn in through an appropriate media to establish sterility of the drawn in air that has passed through the appropriate media. Each of the fluid reservoirs 402, 404, 406 may also have features, dimensions and materials which are similar to or the same as those of the fluid reservoir embodiments 18 discussed above. The fluid volumes 122, 122ʹ, 122ʺ of each of the respective fluid reservoirs 402, 404, 406 may also be disposed in fluid communication with a reservoir selector assembly 410 which is configured to selectively establish fluid communication between a single one of the fluid volumes 122, 122ʹ, 122ʺ of one of the respective fluid reservoirs 402, 404, 406 and the inlet port 42 of a pump chamber assembly 407.

The reservoir selector assembly embodiments discussed and illustrated herein may include certain features and configurations, however, for certain medical pump system embodiments the reservoir selector assembly may include any suitable form that performs the desired fluid volume selection function. For example, certain reservoir selector assembly embodiments may include any suitable valve arrangement that is configured to selectively provide an exclusive fluid conduit from each of a plurality of fluid sources to a pump chamber of a medical pump system. In some instances, a valve arrangement of such reservoir selector assemblies may include indexable rotating fluid selector valves that may be actuated by an actuator which is coupled to a pump chamber actuator with a one way coupling or the like in order to selectively actuate the reservoir selector assembly independent of the pumping function of the pump chamber.

The pump chamber assembly 407 may be configured to controllably pump fluids from the inlet port 42 thereof to the outlet conduit 56 when actuated and may include a configuration similar to or the same as that of the pump chamber assembly 32 discussed above. A pump chamber actuator 408, that may include a cam such as cam shaft 70 or similar element, may be operatively coupled to the pump chamber assembly 407. A motive force actuator such as the motor 84 may be operatively coupled to the pump chamber actuator 408 which may also be operatively coupled to the controller 88. A power source 148 may be operatively coupled to the controller 88, the motor 84 or any other suitable components of the medical pump system 400. The optional transmission 160 may be operatively coupled between the motor 84 and the pump chamber actuator 408 in some instances.

The reservoir selector assembly 410 may include a selector valve assembly 412 and a selector valve actuator 414 that is operatively coupled to the selector valve assembly 412. The selector valve actuator 414 may also be operatively coupled to the pump chamber actuator 408 by a selector valve coupling 416 which may be configured to permit the motor 84 or any other suitable actuator to effectively actuate both the pump chamber actuator 408 and the selector valve actuator 414 in a selective manner. In some cases, the selector valve coupling 416 may include a one-way coupling that drives or otherwise engages in a first direction of rotation and slips in an opposite second direction of rotation. Such a one-way coupling of the valve coupling 416 may include one-way couplings as a spring clutch, clutch bearing, ratchet assembly or the like. The spring clutch allows the motor 84 to actuate the pump chamber actuator 408 and not the selector valve actuator 414 while turning in a first standard pumping direction with the valve coupling 416 slipping and to actuate and drive the selector valve actuator 414 when turning in a second opposite direction with the valve coupling 416 engaged and powering the selector valve actuator 414.

Respective pressure sensors 130, 130ʹ, 130ʺ may be disposed in operative communication with each of the fluid reservoirs 402, 404, 406 by means of fluid conduits 17 and also in operative communication with the controller 88 by appropriate conducting conduits 15. For the embodiment shown, the respective pressure sensors are disposed in fluid communication with the air volumes 24, 24ʹ, 24ʺ by means of fluid conduits 17. The pressure sensors 130, 130ʹ, 130ʺ may be used to monitor the pressure disposed within each of the respective rigid outer containers 20, 20ʹ, 20ʺ of each of the fluid reservoirs 402, 404, 406 with the processor 90 of the controller 88 performing any of the related functions such as fluid volume determination, alarm condition detection etc. For some medical pump system embodiments 400 shown, each fluid reservoir 402, 404, 406 may be individually vented such as by a small orifice (not shown) through the wall of the respective rigid outer shells 20 of each fluid reservoir 402, 404, 406 as well as any other suitable method discussed herein or the like.

For some embodiments, the fluid reservoirs 402, 404, 406, pump chamber assembly 407, and selector valve assembly 412 of the reservoir selector assembly 410 may be included in a reservoir cartridge assembly 420 of the medical pump system 400. For some embodiments, the controller 88, motor 84, power source 148, pump chamber actuator 408, selector valve actuator 414, pressure sensors 130, 130ʹ, 130ʺ and selector valve coupling 416 may be included in an actuator assembly 422 of the medical pump system 400. The schematic representations of the reservoir cartridge assembly 420 and actuator assembly 422 of the medical pump system 400 shown in FIG. 25 are separated from each other by dashed line 424 and may interface with each other in a manner similar to the interaction and interfacing of the reservoir cartridge assembly 12 and actuator assembly 14 discussed above including a latch mechanism 16.

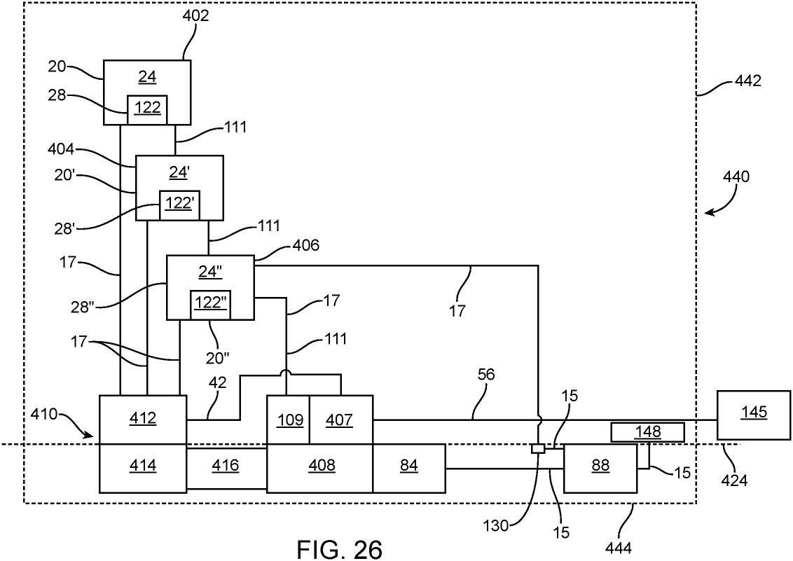

FIG. 26 illustrates a schematic representation of a medical pump system embodiment 440 which may be similar to the medical pump system embodiment 400 discussed above and include any of suitable features, dimensions or materials of medical pump system embodiment 400. An actuator assembly 444 of the medical pump system embodiment 440 may be configured to operate in conjunction with a reservoir cartridge assembly 442 which includes the vent valve assembly 109 operatively coupled to the pump chamber actuator 408. The vent valve assembly 109 may be coupled in fluid communication to the interior volume of the respective rigid outer containers 20, 20ʹ, 20ʺ of the three fluid reservoirs 402, 404, 406 by the common vent conduit pathway 111 as shown which extends and communicates with the respective interior air volumes 24, 24ʹ, 24ʺ of each of the respective rigid outer containers 20, 20ʹ, 20ʺ. For such embodiments, the pressure inside the interior air volumes 24, 24ʹ, 24ʺ as well as the respective fluid volumes 122, 122ʹ, 122ʺ of all three fluid reservoirs 402, 404, 406 will be equal and may be vented at the same time and in the same manner as discussed above with regard to the operation of the vent valve assembly 109.

Alternatively, separate vent conduit pathways 111 for each of the fluid reservoirs 402, 404, 406, which are not in fluid communication with each other, may establish separate fluid communication between each of the respective fluid reservoirs 402, 404, 406 and a vent valve assembly embodiment 109. Such a vent valve assembly embodiment 109 may be configured to vent each fluid reservoir 402, 404, 406 separately and independently.

Although the medical pump system embodiment 440 is shown with three fluid reservoirs 402, 404, 406, any suitable number of multiple fluid reservoirs may be used including 2, 3, 4, 5 or more fluid reservoirs in some cases. Each of the fluid reservoirs 402, 404, 406 may have the fluid volumes 122, 122ʹ, 122ʺ separated from the respective air volumes 24, 24ʹ, 24ʺ by the flexible membrane 28, 28ʹ, 28ʺ and surrounded by the respective rigid outer container 20, 20ʹ, 20ʺ. Fluid volumes 122, 122ʹ, 122ʺ, any or all of these fluid or liquid volumes may contain liquids or suitable gases such as air, inert gases such as argon, helium, or the like. Because the common vent conduit pathway 111 equalizes the pressure within the three fluid reservoirs 402, 404, 406, for the embodiment shown, a single pressure sensor 130 may be disposed in the actuator assembly 444 and coupled in fluid communication with the interior volume of any one of the fluid reservoirs 402, 404, 406, by a fluid conduit 17 and also operatively coupled to the controller 88 by the conducting conduit 15 in order to provide the desired pressure measurement information and functions.

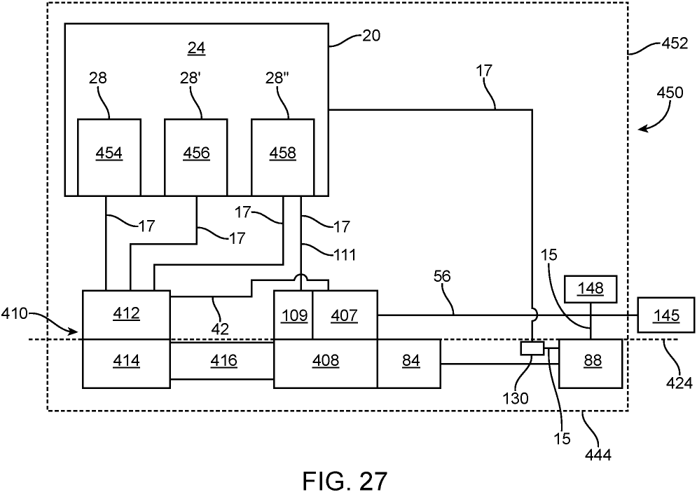

FIG. 27 shows a schematic representation of a medical pump system embodiment 450 which may be similar in some respects to the medical pump system embodiments 400, 440 discussed above. The actuator assembly 444 of the medical pump system embodiment 450 may be the same as or similar to that of the medical pump system 440. However, the reservoir cartridge assembly 452 of this embodiment may include the vent valve assembly 109 operatively coupled to the pump chamber actuator 408 and also coupled in fluid communication to the interior volume of the single rigid outer container 20 which is disposed about a first fluid volume 454, a second fluid volume 456, and a third fluid volume 458, each of the respective fluid volumes 454, 456, 458 being surrounded by a separate respective flexible membrane 28, 28ʹ, 28ʺ.

Each of the fluid volumes 454, 456, 458 may have the same or similar features, dimensions and materials as those of the fluid or liquid volume embodiments 22 or fluid volume embodiments 122 discussed above. Each of the fluid volumes 454, 456, 458 may be separated from the air volume 24 by the optional respective flexible membranes 28, 28ʹ, 28ʺ and surrounded by the rigid outer container 20. Although labeled as fluid volumes 454, 456, 458, any or all of these fluid volumes may contain liquids or suitable gases such as air, inert gases such as argon, helium, or the like. The vent valve assembly 109 is coupled in fluid communication to the interior volume of the rigid outer container 20 by the vent conduit pathway 111. For such embodiments, the pressure inside the fluid volumes 454, 456, 458 will be equal and the interior volume of the rigid outer container 20 may be vented in the same manner as discussed above with regard to the operation of the vent valve assembly 109. Although the medical pump system embodiment 450 is shown with three fluid volumes 454, 456, 458, any suitable number of multiple fluid volumes may be used including 2, 3, 4, 5 or more fluid volumes in some cases.

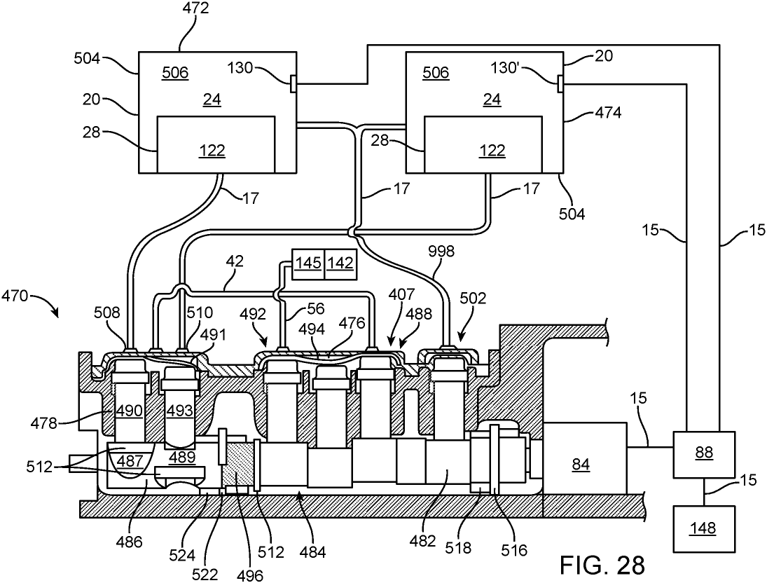

FIG. 28 is a schematic view in partial section of a medical pump system embodiment 470 which is configured for delivering two different fluids from two different fluid reservoirs. Although the medical pump system embodiment 470 is shown with two fluid reservoirs, any suitable number of multiple fluid reservoirs may be used including 2, 3, 4, 5 or more fluid reservoirs in some cases. In some instances, a plurality of different fluids to be delivered to a patient 142 from the medical pump system 470 may include medicaments and other medically useful fluids, such as therapeutic fluids 50 discussed above as well as any suitable gases such as air that might be useful to pump through the system 470. Some such medical pump system embodiments 470 may include wearable pump systems configured for wearing on the body of a patient 142.

In some instances, each of the plurality of therapeutic fluids 50, which may include different therapeutic fluids 50, may be delivered from a corresponding and separate fluid reservoir utilizing a common actuation mechanism such as the pump chamber assembly 407. Pump chamber assembly 407 may, in some instances, be similar to or the same as the pump chamber assembly 32 of the medical pump system 10 discussed above. The pump chamber assembly 407 may be used with a delivery output port or tubing such as the outlet conduit 56 and patient port 145 discussed above and shown in FIG. 28 in order to couple the output of the pump chamber assembly 407 to the patient 142. Such delivery output ports or tubing 56 may have an inner lumen disposed in fluid communication with an interior portion of a patient’s body 142, such as the subcutaneous delivery site 140, beneath the skin 143 of the patient’s body 142 as shown in FIG. 3 and discussed above. Each of the plurality of fluid reservoirs for such pump system embodiments 470 may have features, dimensions and/or materials which are the same as or similar to those of the fluid reservoir 18 discussed above.

Suitable examples of related fluid pump system embodiments and associated delivery output port or tubing embodiments may be found in patent applications U.S. Pat. Application Ser. No. 16/028,256, titled “Medical Pump with Flow Control”, filed Jul. 5, 2018, by P. DiPerna, U.S. Pat. Application Ser. No. 16/520,521, titled “Subcutaneous Access Hub with Multiple Cannula Ports”, filed Jul. 24, 2019, by P. DiPerna et al., and U.S. Pat. Application Ser. No. 15/122,132, titled “Fluid Delivery Pump”, filed Aug. 26, 2016, by P. DiPerna et al., each of which is incorporated by reference herein in its entirety.

Some medical pump system embodiments, such as the medical pump system embodiment 470 of FIG. 28, may be configured to selectively draw therapeutic fluid 50 from a particular fluid reservoir of a plurality of fluid reservoirs, such as a first fluid reservoir 472 or the second fluid reservoir 474. Such a configuration may allow an actuator including the pump chamber assembly 407 of such medical pump system embodiments 470 to move the therapeutic fluid 50 or any other suitable fluid through a common pumping mechanism and out a shared output port or tubing 56.

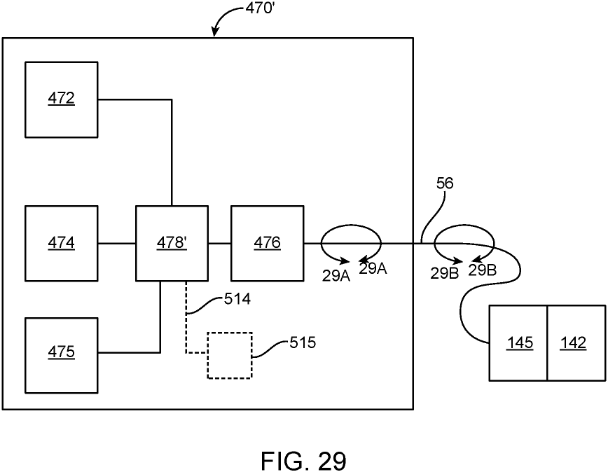

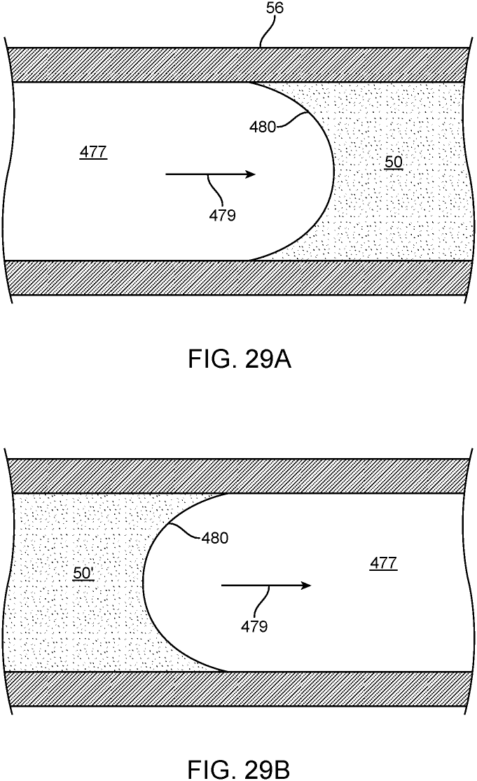

A pump chamber 476 of such medical pump system embodiments 470 may thus be filled with various different therapeutic fluids 50 at different times. In such cases, a residual volume of a previous or primary therapeutic fluid 50 compared to a newly introduced volume of a subsequent therapeutic fluid 50' (see FIGS. 29A and 29B discussed below) may be known and predictable by allowing the controller 88 to use an algorithm to adjust and compensate for the mixing of the previous therapeutic fluid 50 with the subsequent therapeutic fluid selection 50ʹ and the resulting reduced and diminishing concentration of the previous therapeutic fluid 50 over time during delivery of the subsequent therapeutic fluid 50ʹ.

In some cases, the pump chamber 476 of embodiments of the medical pump system 470 shown in FIG. 28 may be filled or otherwise supplied with a diluent drawn from one of the plurality of fluid reservoirs 472 or 474 to provide a rinse, thereby flushing a therapeutic fluid 50 from the pump chamber 476 of the pump mechanism 407. Such a rinsing procedure may be used to enable a clean introduction of a subsequent alternative therapeutic fluid 50ʹ without mixing of the subsequent therapeutic fluid 50ʹ with the previous therapeutic fluid 50. This may be particularly useful for medical pump systems 470 that include more than two fluid reservoirs 472, 474, including medical pump system embodiments 470 having three, four, five or more fluid reservoirs that are each operatively and selectively coupled to the pump mechanism 407 of the medical pump system 470.

Diluents suitable for such rinsing procedure embodiments may be formulated to have no biologically active components in some cases in order to avoid interaction with any of the previously delivered or subsequently delivered therapeutic fluids 50, 50ʹ. Such a configuration may serve to minimize or eliminate the mixing of two different sequential therapeutic fluids 50, 50ʹ within the pump chamber 476 and/or associated output port 56 or tubing and/or delivery port 145. In some cases, such a diluent fluid used for flushing or rinsing may include saline, dextrose or the like. For some embodiments, the internal features, dimensions and/or materials of the pump mechanism 407 used for medical pump system embodiments 470 having multiple fluid reservoirs, such as the medical pump system embodiment 470 shown in FIG. 28, may be the same as or similar to those pump chamber assembly embodiments 32 of medical pump system embodiments 10 having a single fluid reservoir 18, such as those which are discussed in in U.S. Pat. Application Ser. No. 17/111,396, titled “Rotary Microfluidic Medical Pump”, filed Dec. 3, 2020, by P. DiPerna et al., which is incorporated by reference herein in its entirety.

In addition to the use of liquid diluents such as saline, dextrose or the like, other non-liquid materials may be used to purge, rinse or otherwise effectively partition the pump chamber 476 and tubing 56 with respect to the delivery of differing sequential therapeutic fluids 50, 50ʹ. In particular, a small separation formed by the presence of a gas such as air or inert gases such as Argon, Helium or the like disposed between each of two different sequential therapeutic fluids 50, 50ʹ may be used to facilitate the use of a common pump chamber 476 and a common tubing 56 and associated subcutaneous or intravenous needle or cannula for sequential delivery of two or more different therapeutic fluids 50, 50ʹ. In such cases, the gap formed by the presence of the gas disposed within the pump chamber 476 and/or the inner lumen of tubing 56 or elsewhere in the system may be used to separate the two or more different sequential therapeutic fluids 50, 50ʹ prevent any significant mixing therebetween.

For some such embodiments, after a first therapeutic fluid 50 is delivered to the patient 142, a reservoir selector assembly 478ʹ which is configured to select from three different fluid reservoirs 472, 474 and 475 may be rotated or otherwise actuated to change the reservoir source from the first fluid reservoir 472 to a third fluid reservoir 475, of a medical pump system 470ʹ as shown in FIG. 29. FIG. 29 shows a schematic representation of a basic medical pump system embodiment 470ʹ that may be the same as or similar to the medical pump system embodiments 400, 440, 450, 470 and includes the third fluid reservoir 475 and a suitably adapted reservoir selector assembly 478ʹ. The third fluid reservoir 475 may have any of the same features, dimensions or materials as those of either the first fluid reservoir 472 or second fluid reservoir 474.

The interior volume of the third fluid reservoir 475 may be filled with air that may optionally be filtered in order to maintain the biocompatibility and/or sterility of the filtered air 477. In some cases, the filtered air 477 within the interior volume of the third fluid reservoir 475 may be filtered with a sterility filter having a pore size of about 0.22 µm or smaller to essentially achieve sterility of the filtered air 477 disposed therein. In the alternative to a third fluid reservoir 475, filtered air 477 may be introduced directly into a third selector valve assembly of the reservoir selector assembly 478ʹ without a third fluid reservoir 475 by placing a suitable sterility filter 515 over an inlet conduit 514 of the third selector valve as shown by the dashed lines in FIG. 29. The sterility filter 515 may include a filter with a pore size of about 0.22 µm or less as discussed above.

An illustration of the separation between sequential therapeutic fluids 50, 50ʹ and a bolus of a gas such as air during sequence of delivering a first therapeutic fluid 50 into the inner lumen of the outlet conduit 56 followed by a bolus of filtered air 477 and then a bolus of a second therapeutic fluid 50ʹ is shown in FIGS. 29A and 29B. FIG. 29A shows the separation and partitioning between the first therapeutic fluid 50 and the second therapeutic fluid 50ʹ by the filtered air 477 during delivery in the direction as indicated by arrows 479. A meniscus 480 may be formed at the boundary between the filtered air 477 and the trailing edge of the first therapeutic fluid 50 and the boundary between the leading edge of the second therapeutic fluid 50ʹ and the filtered air 477. The presence of such menisci may be useful in efficiently purging the first therapeutic fluid 50 from the outlet conduit 56 and other associated lumens and chambers, such as the pump chamber, prior to passage and delivery of the second therapeutic fluid 50ʹ. Surface tension of the therapeutic fluids 50, 50ʹ may also play a role in efficiently purging the first therapeutic fluid 50 from the pump chamber 476 and outlet conduit 56 as well as other conduit structures.

For some embodiments, a full single cycle of the pump chamber 476 may be configured to pump a volume of about 5 µl of air from the third fluid reservoir 475 into the inner lumen of the tubing 56 before the reservoir selector assembly 478ʹ is again actuated to change the reservoir source to the second fluid reservoir 474 which contains a second therapeutic fluid 50. In some cases, the pump chamber 476 first completes the pushing of the first therapeutic fluid 50 into the tubing 56, then pumps the small quantity or bolus of air from the third reservoir 475 (or optionally from the inlet conduit 514) into the outlet conduit tubing 56. The pump chamber 476 may then start to draw from the second fluid reservoir 474 and deliver the second therapeutic fluid 50ʹ into the tubing 56 and ultimately through the associated cannula and into the patient’s body 142.

Since the volume of the pump chamber 476, which may be about 5 µl per cycle, and the volume of the tubing 56 and associated needle/cannula, which may be about 10 µl in some cases, is known, such a configuration allows for delivery of the first therapeutic fluid 50 to the patient 142 with a subsequent pause in the delivery of the first therapeutic fluid 50 as the air bolus is delivered from the third fluid reservoir 475. During delivery of the air bolus, the first therapeutic fluid 50 is allowed to disperse from the injection site 140 prior to delivery of the second therapeutic fluid 50ʹ. Such an arrangement and method may provide a small, efficient, cost effective pump system embodiments 470ʹ as shown in FIG. 29 that can deliver a plurality of different therapeutic fluids 50, 50ʹ without any significant mixing therebetween.

Although small quantities of each sequentially delivered therapeutic fluids 50, 50ʹ may coat the inner lumen of the tubing 56 and pump chamber 406 to a certain extent, as discussed above and illustrated in FIGS. 29A and 29B, such sequentially delivered therapeutic fluids 50, 50ʹ tend to be pushed through the pump chamber 406 and tubing 56 by the meniscus 480 of the air/liquid interface. In addition, the surface tension characteristics of the inner lumen of the tubing 56 and pump chamber 406 tend to minimize these residual materials and the subsequent quantities of intermixed therapeutic fluids 50, 50ʹ may generally be insignificant in most circumstances.

Some method embodiments for use of the medical pump system embodiment 470 shown in FIG. 28 may include techniques to dispense a plurality of therapeutic fluids 50, 50ʹ or any other suitable fluid using the single pump chamber assembly 407 including the pump chamber 476 to both deliver therapeutic fluid 50 (as well as other non-therapeutic fluids such as diluents and the like) and select a fluid source from a plurality of fluid sources such as fluid reservoirs (for example, about 2 reservoirs to about 3, 4, 5 or more reservoirs in total) with a reservoir selector assembly such as the reservoir selector assembly embodiment 478 shown in FIG. 28.

The pumping process may include a rotation of one or more cams, such as cam shaft 482, which may be operatively coupled to the pump chamber assembly 407. The cam shaft 482 may be similar to or the same as cam shaft 70 discussed above in some cases. The cam shaft 482 may be so rotated by a motor, such as the motor 84 discussed above, to move the therapeutic fluid 50 through the pump chamber 476 and outlet conduit 56. The rotation of the motor 84 may be directed by a controller, such as the controller 88, which is operatively coupled to the motor 84 as well as a power source, such as power source 148 discussed above. The basic pumping operation of the pump chamber assembly 407 may be carried out by these components in the same or similar manner as is discussed above and illustrated in FIGS. 14A, 14B and 14C.

For medical pump system embodiments 470 that include a plurality of fluid reservoirs 472,474, a cam assembly 484 that includes the cam shaft 482 may be used as shown in the embodiment of FIG. 28. The cam assembly 484 may include a primary pump cam that is configured to sequentially position the valves and displacement diaphragm associated with the pump chamber assembly 407 for moving therapeutic fluid 50 therethrough. The cam assembly 484 also includes a cam segment 486 that is configured to sequentially and controllably position valves 508, 510 associated with the reservoir selector assembly 478 in order to select a source of therapeutic fluid 50 or any other suitable fluid from the plurality of fluid reservoirs 472, 474.

Again, for the medical pump system embodiment 470 shown in FIG. 28, the cam segment 486 is operatively coupled to the cam shaft 482 with a one way selective coupling. With such a coupling, if the primary pump cam shaft 482 is rotated by the rotational actuator (e.g. the gear motor 84) in a standard rotation direction, the pump chamber assembly mechanism 407 that may include the pump inlet valve 488, which may be the same as or similar to the inlet valve assembly 75 discussed above, pump outlet valve 492, which may be the same as or similar to the outlet valve assembly 79 discussed above, displacement diaphragm 494, which may be the same as or similar to the resilient displacement membrane 64 discussed above, and primary pump cam 482 will pump fluid from the selected fluid reservoir (either 472 or 474) and out of the outlet conduit 56 in a manner the same as or similar to that discussed above and shown in FIGS. 14A, 14B and 14C. In this standard pumping direction, the one way coupling will slip and allow the cam segment 486 to remain stationary.

If the gear motor 84 is reversed, the one way coupling which may include a selector valve coupling (such as the selector valve coupling 416 discussed above in the form of a spring clutch 496 or the like of the reservoir selector assembly 478) may engage the extension or subcomponent 486 of the cam assembly 484 (that may be referred to as the cam segment 486 of the cam assembly 484). Rotation of the cam segment 486 in the reverse rotation direction is configured to position the cam lobes, including a first cam lobe 487 and a second cam lobe 489, of the cam segment 486 and associated respective valves 508, 510 of the reservoir selector assembly 478 so as to close all of the valves to non-selected fluid reservoirs 472, 474 and open the valve of the selected fluid reservoir 472, 474 to make the contents and internal volume of the selected fluid reservoir available for flow through the pump mechanism 407.

For example, for the position of the cam segment 486 shown in FIG. 28, the first cam lobe 487 is fully extended against the first selector pushrod 490 so as to seal the resilient selector valve membrane 491 against the inlet port of the first selector valve assembly 508 so as to prevent fluid communication between the inlet port 42 of the pump chamber 476 and the first fluid reservoir 472. In this same position as shown, the second cam lobe 489 is in a retracted position allowing the resilient selector valve membrane 491 and second selector pushrod 493 to retract away from the inlet port of the second selector valve assembly thereby permitting fluid communication between the second fluid reservoir 474 and the inlet port 42 of the pump chamber 476.

In order to control the pressure within each of the fluid reservoirs 472, 474, during delivery of fluids by the pump chamber assembly 407, a vent conduit 498 (which may be the same as or similar to the vent conduit 111 discussed above) may be disposed in fluid communication between a vent valve assembly 502 (which may be the same as or similar to the vent valve assembly 109 discussed above) and the respective interior volumes of the fluid reservoirs 472, 474 as shown in FIG. 28. The respective interior volumes of the fluid reservoirs 472, 474 are in fluid communication with each other by virtue of the common vent conduit 498, similar to the arrangement shown for the medical pump system embodiment 440 discussed above, and, as such, are at the same pressure. However, any other suitable arrangement for the vent conduit 498 and associated fluid reservoirs 472, 474 may be used. The vent valve assembly 502 is configured to be actuated by rotation of the cam assembly 484 and selectively vent the fluid reservoirs 472, 474 in order to release negative pressure build up due to dispensing of therapeutic fluids 50 or the like therefrom as well as making volume determinations etc. as discussed above.

With further regard to the fluid reservoir selection process by the reservoir selector assembly 478, in some cases the reverse rotation of the cam segment 486 may be used to selectively open a single inlet valve from the first reservoir 472 while all other reservoir valves (such as the inlet valve associated and in fluid communication with the second fluid reservoir 474) remain shut off or closed. Thereafter, rotation of the gear motor 84 and cam shaft 482 in the standard direction now dispenses fluid from the selected fluid reservoir, through the pump chamber 476, and out the outlet conduit 56 to the patient’s body 142 through a single infusion set tubing or cannula 145 as the spring clutch 496 freewheels without actuating the cam segment 486.

As discussed briefly above, the respective pressure sensors 130 may be used to measure the pressure within one or more of the fluid reservoirs 472, 474 in order to monitor a variety of states of the medical pump system 470 including the amount of fluid dispensed from each fluid reservoir 472, 474, the amount of fluid remaining in each of the fluid reservoirs 472, 474, or detection of a blockage in the outlet conduit 56 or associated conduits etc. In some cases, a first pressure sensor 130 may be disposed in fluid communication with and configured to measure pressure within the interior volume of the first fluid reservoir 472 and the second pressure sensor 130ʹ may be disposed in fluid communication with and configured to measure the pressure within the second fluid reservoir 474 as shown in the embodiment of FIG. 28. This arrangement may be particularly useful for medical pump system embodiments 470 that do not have a common vent conduit 498 in fluid communication with both the fluid reservoirs 472, 474.

However, in some instances, a single pressure sensor 130 may be operatively coupled to both the first fluid reservoir 472 and the second fluid reservoir 474 and configured to measure pressure within either the first fluid reservoir 472 or the second fluid reservoir 474 as is illustrated in the medical pump system embodiment 440 shown in FIG. 26. For medical pump system embodiments 470 that include fluid reservoirs 472, 474, having compliant or soft sided flexible membranes 28 disposed about all or part of the fluid volume 122 of the fluid reservoir, the additional rigid outer container 20 which may take the form of a fluid tight shell may be disposed about the compliant membrane 28. The rigid outer container 20 may be configured to form a rigid chamber 504 having an interior volume 506 and the pressure sensor (or sensors) 130, 130ʹ may be disposed in fluid communication with the interior volume 506 of the rigid chamber 504.

Such a configuration allows for pressure measurement within both the rigid chamber 504 and within the fluid volume 122 of the compliant membrane 28 that contains the associated therapeutic fluid 50. For those embodiments wherein only a single pressure sensor 130 is used, a single rigid outer container 20 may be disposed about both the first fluid reservoir 472 and second fluid reservoir 474 with the single pressure sensor 130 being operatively coupled to the interior volume of the single rigid outer container 20 similar to the arrangement shown for the rigid outer container 20 and fluid volumes 454, 456, 458 of the medical pump system 450 shown in FIG. 27.

Additionally, some embodiments of the medical pump system 470 shown in FIG. 28 may include the controller 88 that is configured to process an algorithm that is calibrated based on the mixing characteristics of multiple therapeutic fluids 50, 50ʹ (or non-therapeutic fluids such as diluents) within the common pump chamber 476 so that switching between different fluids from different fluid reservoirs 472, 474, is completed in a predictive fashion, controlling the dispensing quantity of the previously delivered fluid and the timing to introduce the new desired fluid. The effect on relative concentrations of the two sequential fluids being dispensed during such a transition may also be controlled. In addition, for embodiments wherein use of a diluent is contemplated to simplify the changeover of fluids, such an algorithm may be involved or otherwise processed by the controller 88 in order to adjust for a lack of the subsequent new subsequent therapeutic fluid 50ʹ as the diluent volume is flushed out of the pump chamber 476 and output tubing 56 without any effect versus the previous active medication process.

For some embodiments, the volume of the pump chamber 476 may be about 5 µl to about 50 µl and may also be scaled based on a desired resolution of liquid output and acceptable mixing concentration of different fluids. The volume of the respective fluid volumes 122 and air volumes 24 of the fluid reservoirs 472, 474, may be independent of the volume of the pump mechanism embodiments 407 and may be sized accordingly to the user needs for a specific therapeutic fluid 50.

As discussed above, embodiments of the medical pump system 470 may be configured to selectively couple multiple fluid reservoirs 472, 474 or other sources of fluids, such as a source of a gas including filtered air, to the pump inlet valve 488 of the common pump chamber 476 using an array of fluidic valves of the reservoir selector assembly 478. This selector valve array of the reservoir selector assembly 478 may include at least one selector valve for each separate fluid reservoir 472, 474. The two fluid reservoirs 472, 474 and their respective associated first selector valve assembly 508 and a second selector valve assembly 510 are illustrated as an example in the embodiment of FIG. 28, but up to three, four or more fluid reservoirs are contemplated. In some cases, such a reservoir selector assembly 478 including the selector valve assembly 508, 510 may be controlled with the cam segment 486 that may be coaxially coupled with the primary pump cam shaft 482 and its rotational actuator such as the gear motor 84 with a one way coupling or the like.

The cam segment 486 may include one lobe, such as the respective first cam lobe 487 and second cam lobe 489, for each valve of the selector valve assembly 508, 510 of the reservoir selector assembly 478. Each lobe 487, 489 of the cam segment 486 may be configured or otherwise “timed” such that only one selector valve assembly can be open at a time while the other valves associated with non-selected fluid reservoirs 472, 474 are closed. In some cases, such as with the medical pump system embodiment 470 shown in FIG. 28 that includes two fluid reservoirs 472, 474, each lobe 487, 489 of the cam segment 486 may be configured to lift over a rotation of about 90 degrees. For example, the first cam lobe 487 of the first valve of the first selector valve assembly 508 of the reservoir selector assembly 478 may be timed so as to be open at 0 degrees, closed at 90 degrees, open at 180 degrees and closed at 270 degrees. The second cam lobe 489 of the second valve of the second selector valve assembly 510 of the reservoir selector assembly 478 may be timed in an opposite pattern, i.e., closed at 0 degrees, open at 90 degrees, closed at 180 degrees and open at 270 degrees.

For medical pump system embodiments 470 having more than two fluid reservoirs 472, 474, for example a medical pump system embodiment 470 having four fluid reservoirs including a reservoir selector assembly 478 having four respective selector valves and four respective cam lobes of the cam segment 486, each of the respective selector valves may be open at only one rotational position of the cam segment 486 rather than at two as discussed above. When any one of the four selector valve assemblies of such a reservoir selector assembly 478 is open, the respective lobes of the cam segment 486 of such an embodiment may be timed such that all three other selector valve assemblies are closed. In some cases, for such a cam segment embodiment 486, the diameter of the cam segment 486 may be at least about 4 mm in nominal diameter.

In some instances, the cam segment 486 may be rotationally coupled to the primary pump cam shaft 482 utilizing the selector valve coupling 416 in the form of the one way coupling such as the spring clutch 496 (sometimes referred to as a frictional clutch) of the reservoir selector assembly 478. This spring clutch 496 may be configured such that when the primary pump cam shaft 482 turns in a standard forward “dispense” or “pump” direction, the spring clutch 496 slips or freewheels and the cam segment 486 remains stationary (disengaged). When the primary pump cam shaft 482 turns in the opposite “select” direction (backwards), the spring clutch 496 engages to prevent slipping and the cam segment 486 is driven in the same opposite direction with the primary pump cam shaft 482. This reverse rotation and associated reverse rotation of the cam segment 486 may be used for opening a valve 508, 510 for the specific selected fluid reservoir 472, 474 to draw fluid from. For some embodiments, when the primary pump cam shaft 482 rotates in this backwards direction the pump mechanism 407 may be configured with the outlet valve 492 closed so there is no fluid transfer to the patient’s body 142 during the reservoir selection process and operation of the cam segment 486.

When the primary pump cam shaft 482 is turning in the standard forward dispense direction, there may be a small amount of friction at the spring clutch 496 that compels the cam segment 486 to follow the primary pump cam shaft 482 in the same standard rotational direction. To prevent this undesired motion of the cam segment 486, the cam segment 486 of the reservoir selector assembly 478 may have one or more ratchetting features 512 that prevent this forward motion while allowing the cam segment 486 to be driven in the backward direction as required. Such ratcheting features 512 may include a step or shelf structure on the cam segment 486 designed to engage with an associated valve piston/pushrod, eliminating the need for additional parts such as a pawl or catch.

The primary pump cam shaft 482 may include a first fiducial or rotary flag 516 as shown in FIG. 28 that is monitored by a first stationary optical detector 518 (or any other suitable type of detector), providing information to the controller 88 about the rotational position of the primary pump cam shaft 482. The cam segment 486 of the reservoir selector assembly 478 may also include a second fiducial flag 522 that is monitored by a second stationary optical detector 524 (or any other suitable type of detector), providing information to the pump controller 88 about the rotational position of the cam segment 486 which fluid is selected by the reservoir selector assembly 478. Such optical detectors 518, 524 may be replaced by other suitable detector configurations including a contact switch, capacitive sensor, IR sensor, photo reflective, or similar types of position detector mechanisms.

In addition, embodiments of the entire medical pump system 470 shown in FIG. 28 may be configured to operate with the motor 84 and cam shaft 482 in parallel with a spur gear or belt drive setup (not shown). Such a configuration may reduce an overall length of the pump mechanism 407. An alternative configuration such as orthogonal arrangement between the cam shaft 482 and motor 84 is possible with beveled gears or an optional flexible coupler (not shown). A rotary force to be coupled to the cam shaft 482 may be provided by a rotary DC or stepper motor. Alternatively a ratcheting mechanism could be designed to drive the cam shaft 482 in two rotational directions.

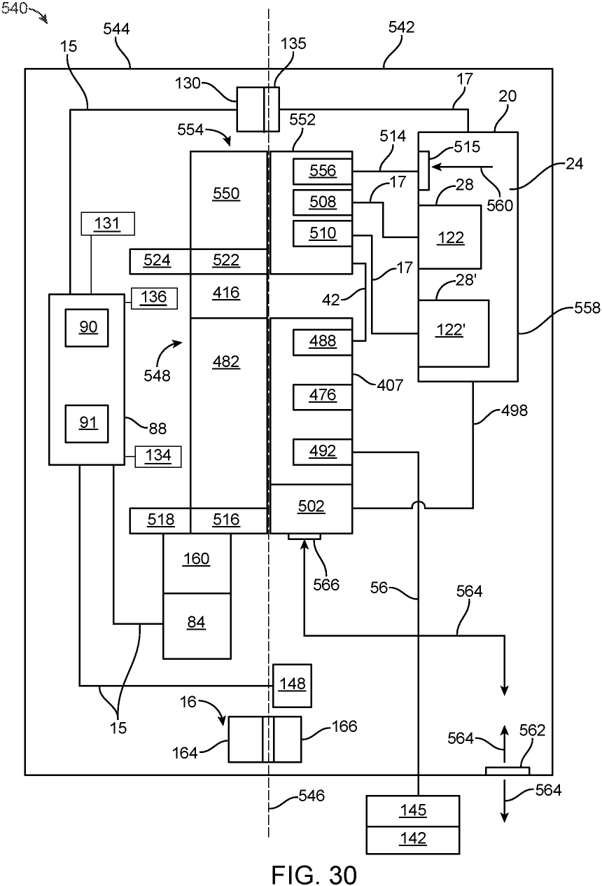

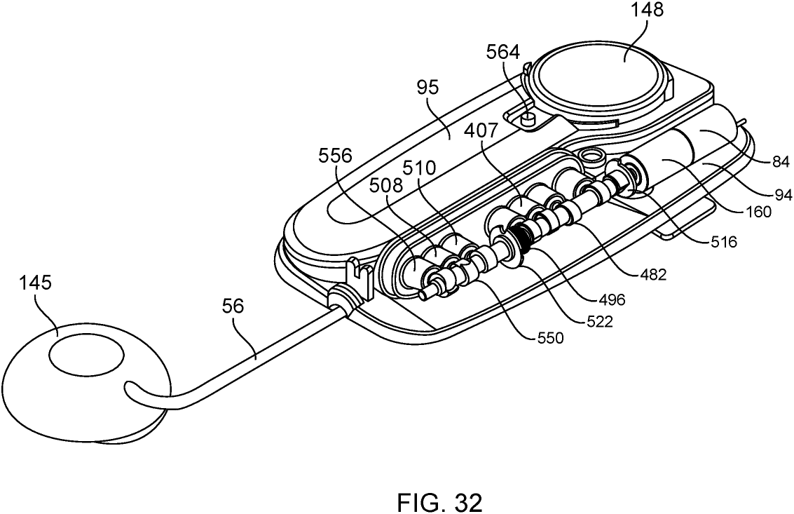

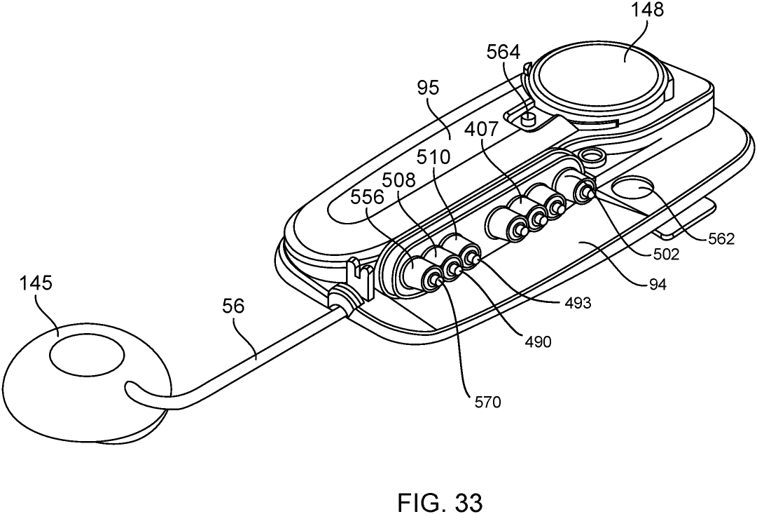

Referring to FIGS. 30-40, a medical pump system embodiment 540 is illustrated that includes a reservoir cartridge assembly 542 and an actuator assembly 544 configured to be operatively coupled together in a releasable fashion. The medical pump system 540 may include many of the same or similar features, dimensions and materials as those of the medical pump system embodiments 12, 400, 440, 450, and 470 discussed above. The medical pump system 540 includes a three input reservoir selector assembly 554 operatively coupled to a first fluid volume 122, a second fluid volume 122ʹ and the filtered air conduit 514 and sterility air filter 515. The filtered air conduit 514 and sterility filter 515 may be used for the introduction of filtered air 477 into the pump chamber 476 of the pump chamber assembly 407 and subsequently to the outlet conduit 56 without the need to store the filtered air 477 in its own separate reservoir. As discussed above, such introduction of filtered air 477 into the pump chamber 476 and outlet conduit 56 may be used to separate the delivery of sequential therapeutic fluids 50, 50ʹ through the pump chamber 476, outlet conduit 56, the port 145 and eventually to the patient 142.

FIG. 30 is a schematic representation of the medical pump system embodiment 540 with the functional and physical interface between the reservoir cartridge assembly 542 and actuator assembly 544 represented by the dashed line 546. The medical pump system embodiment 540 is configured for delivering two different fluids from the two different fluid volumes 122, 122ʹ of a fluid reservoir assembly 558 which includes the fluid volumes 122, 122ʹ which are surrounded by respective flexible membranes 28, 28ʹ. The flexible membranes 28, 28ʹ are enclosed within the interior volume of the rigid outer container 20. The medical pump system 540 is also configured to deliver filtered air 477 into the pump chamber 476 and out the outlet conduit 56. The filtered air 477 is drawn from the inlet conduit 514 which is coupled to the air filter 515. Although the medical pump system embodiment 540 is shown with two fluid volumes 122, 122ʹ and a filtered air inlet conduit 514, any suitable number of multiple fluid volumes or purified or filtered gas sources may be used including 3, 4, 5 or more fluid volumes and/or purified gas sources in some cases.

In some instances, the plurality of different fluids to be delivered by the medical pump system 540 may include medicaments and other medically useful fluids, such as therapeutic fluids 50 discussed above. Some such medical pump system embodiments 540 may include wearable pump systems configured for wearing on the body of a patient 142. In some instances, each of the plurality of therapeutic fluids 50 may be delivered from a corresponding and separate fluid volume of the fluid reservoir assembly 558 utilizing a common actuation mechanism such as the pump chamber assembly 407 and cam assembly 548. The pump chamber assembly 407 discussed above may, in some instances, be similar to or the same as the pump chamber assembly 32 of the medical pump system 10 discussed above.

Such a pump chamber assembly 407 and a cam assembly 548 may be used in conjunction with the delivery output port or tubing such as the outlet conduit 56 and patient port 145 discussed above and shown in FIG. 28. Such delivery output ports or tubing 56 may have an inner lumen disposed in fluid communication with an interior portion of a patient’s body 142, such as the subcutaneous delivery site 140, beneath the skin 143 of the patient’s body 142 as shown in FIG. 3. Each of the plurality of fluid volumes 122, 122ʹ of a fluid reservoir assembly 558 for such pump system embodiments 540 may have features, dimensions and/or materials which are the same as or similar to those of the fluid reservoir embodiments 18, 402, 404, 472, 474 discussed above. In addition, medical pump system embodiments 540 may include user interface devices, methods and protocols which are the same as or similar to those of the medical pump system embodiment 10 discussed above. Thus, medical pump system embodiments 540 may include a control button 134, priming button 136, pressure sensor 130, temperature sensor 131 and the associated controller 88 that is configured to be operatively coupled to these buttons 134, 136 and sensors 130, 131 as discussed above with regard to the medical pump system embodiment 10. Medical pump system embodiments 540 may also include reservoir filling options that are suitable for such multiple fluid designs. In particular, the fluid volumes and/or reservoirs of the multiple fluid medical pump system embodiments 400, 440, 450, 470, 540 discussed herein may be filled by any suitable means such as through a fill port such as the fill port 175 discussed above or they may be prefilled during assembly and manufacture or some appropriate combination thereof. In some cases, one or more of the multiple fluid volumes a particular medical pump system embodiment discussed herein may be prefilled while one or more other fluid volumes of the same medical pump system embodiment may be filled through a fill port such as fill port 175 discussed above.

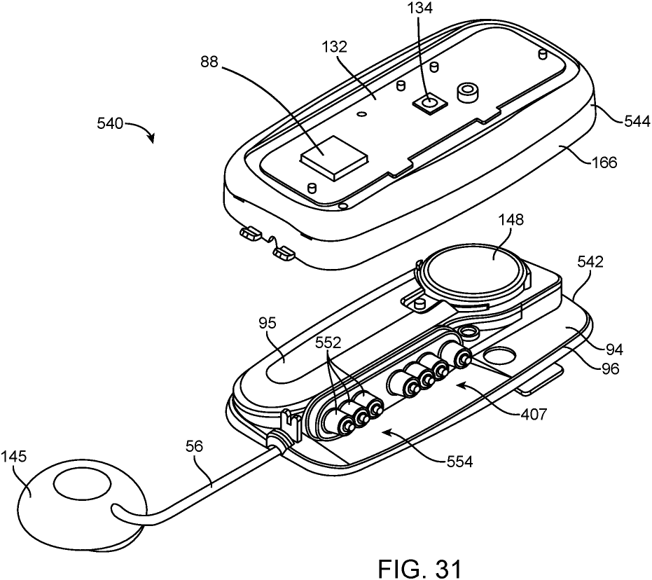

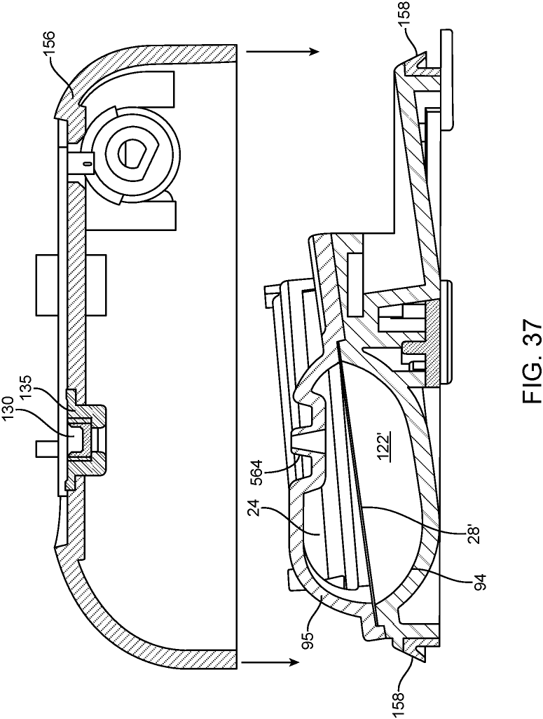

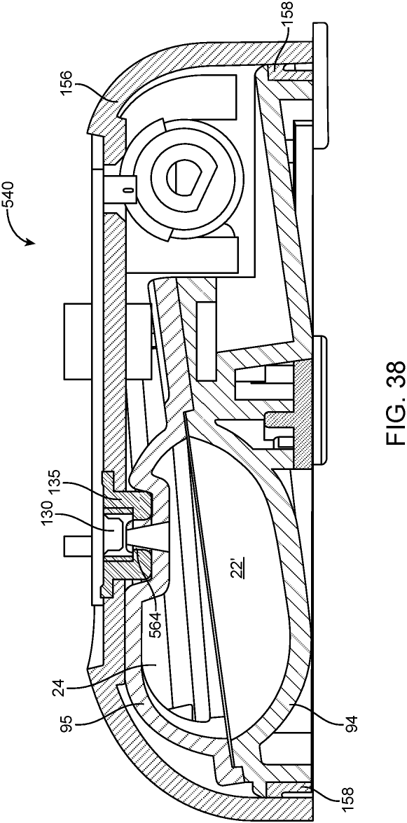

The reservoir cartridge assembly 542 includes a reservoir base 94 having an outer perimeter 96 which may be configured to seal to an outer shell 156 of the actuator assembly 544 as shown in FIG. 31 with an outer shell seal 158 as shown in FIGS. 37 and 38 in a manner discussed above with regard to the medical pump system embodiment 10. The reservoir cartridge assembly 542 and actuator assembly 544 may also be releasably coupled together by a latch mechanism 16 including a latch post 164 and latch spring 166 as shown in FIG. 30 and discussed above with regard to the medical pump system 10 except that the latch post 164 of the medical pump system 540 may include a seal in order to maintain a seal between the outer perimeter 96 of the reservoir base 94 and the outer shell 156 of the actuator assembly. This seal may be useful in order to assure that all vent air that enters or leaves the enclosure formed by the reservoir base 94 and outer shell 156 passes through an air filter 562 as shown in FIG. 30. The air filter 562 may be a hydrophobic high efficiency air filter that allows vent air into and out of the enclosure of the assembled medical pump system 540 as shown by arrows 564 while preventing liquids such as liquid water from entering the enclosure.

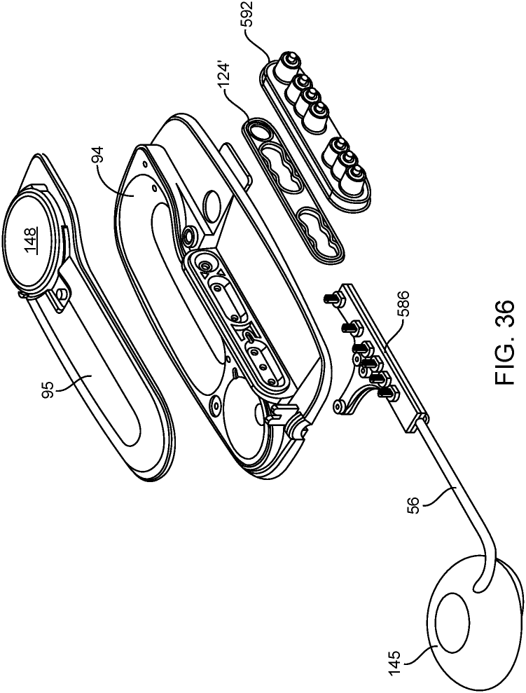

The reservoir cartridge assembly 542 includes the fluid reservoir assembly 558 which has an enclosure formed by the reservoir base 94 and fluid reservoir cover 95 as shown in FIG. 36 and which is represented by the rigid outer container 20 in the schematic depiction of FIG. 30. Within the rigid outer container 20 formed by the reservoir base 94 and fluid reservoir cover 95 are flexible membranes 28, 28ʹ disposed over and respectively sealing fluid volumes 122, 122ʹ. The fluid reservoir cover 95 also has the sterility air filter 515 disposed thereon and over an aperture in the fluid reservoir cover 95 which is disposed in operative and fluid communication with the inlet conduit 514. The inlet conduit 514 is also in fluid communication with a third selector valve assembly 556 of the reservoir selector assembly 554. The third selector valve assembly 556 is configured to select a source fluid for the pump chamber assembly 407. For some embodiments, such a configuration may assure that any sterilized air 477 that is introduced into the pump chamber 476 of the pump chamber assembly 407 must first pass through the hydrophobic air filter 562 on the reservoir base and then through the sterility air filter 515 from within the air volume 24. This provides sterile water free air to the pump chamber 476 for purging, rinsing or other suitable purposes.

In order to monitor the pressure within the air volume 24 of the fluid reservoir assembly 558, the pressure sensor 130 is disposed on the actuator assembly 544 and disposed in operative and fluid communication with the air volume 24 of the fluid reservoir assembly 558. The fluid communication is indicated generally in FIG. 30 by the fluid conduit 17. For the medical pump system embodiment 540 as shown in FIGS. 37 and 38 the fluid communication represented by the fluid conduit 17 in FIG. 30 includes an elastomeric pressure conduit boot 135 disposed on the actuator assembly 544 and a mating nipple 564 disposed on the fluid reservoir cover 95. FIG. 37 shows these mating structures 135, 564 disposed in a separated configuration and in a mated and sealed configuration in FIG. 38. Such a mated and sealed configuration provides a fluid tight conduit between the pressure sensor 130 and the air volume 24. The pressure sensor 130 is also disposed in operative communication with the controller 88 with the conducting conduit 15. The controller 88 is disposed on the actuator assembly 544 and may also include the processor and memory 91. An electrical power source 148 may also be disposed in operative communication with the controller 88 with a conducting conduit 15. For the embodiment shown, the electrical power source 148, which may include a battery, is disposed on the reservoir cartridge assembly 542.

The reservoir cartridge assembly 542 also includes the selector valve assembly 552 which is operatively and releasably coupled to an actuator in the form of the cam segment 550 which is disposed on the actuator assembly 544. The selector valve assembly 552 includes the first selector valve assembly 508, second selector valve assembly 510 and third selector valve assembly 556. The selector valve assembly 552 has an outlet port disposed in fluid communication with the inlet port 42 of the pump chamber assembly 407. The pump chamber assembly 407 includes the pump inlet valve 488, pump outlet valve 492 and pump chamber 476. The pump outlet valve 492 may be disposed in fluid communication with the outlet conduit 56 which, in turn, is disposed in fluid communication with the port 145 and patient 142. A vent valve assembly 502 is also included on the pump chamber assembly 407. The vent valve assembly 502 is disposed in fluid communication with the air volume 24 of the fluid reservoir assembly 558 and also includes a vent inlet port 566 which is in fluid communication with the enclosure formed between the reservoir cartridge assembly 542 and actuator assembly 544. The selector valve assembly 552 and the pump chamber assembly 407 may, in some cases, include a valve housing 592 that houses and guides the various pushrods of the respective valve assemblies as well as other structures.

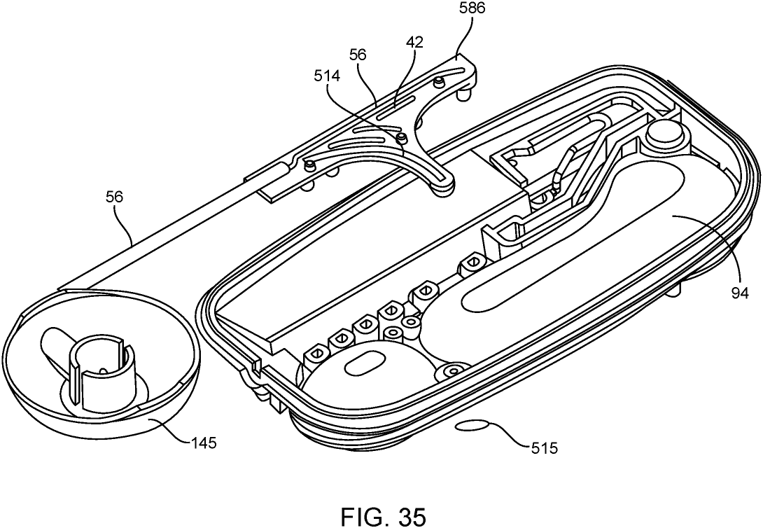

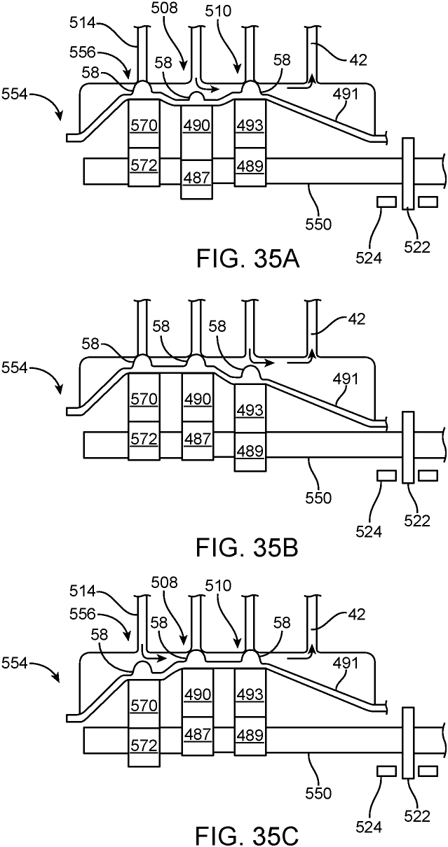

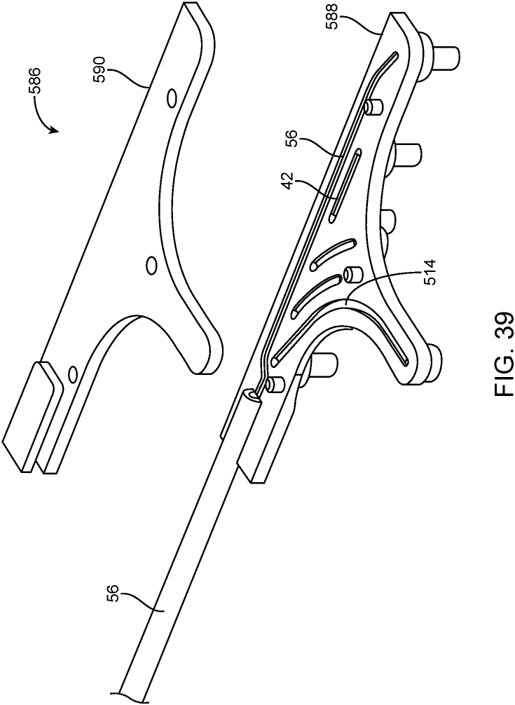

The actuator assembly 544 further includes the cam assembly 548 which includes the cam shaft 482 coupled to the cam segment 550 by a selective coupling such as a one way coupling like the selector valve coupling 416. The cam assembly 548 is operatively coupled to a motive actuator in the form of the motor 84 by an optional transmission 160. The cam assembly is configured to releasably and operatively couple to the pump chamber assembly 407 and selector valve assembly 552 in order to controllably operate their functions. For some embodiments of the medical pump system 540, a flow manifold 586 may be used to fully or partially form the various fluid conduits 17 shown in FIG. 30 and allow for more efficient manufacturing of the medical pump system embodiments 540. Referring to FIGS. 35 and 39, the flow manifold 586 may be made in a first clam shell type half 588 and a second clam shell type half 590 in order to simplify the manufacture of the flow manifold 586. For some embodiments, the first half 588 may include various channels including a section of the outlet conduit 56, the inlet conduit 42, the filtered air inlet conduit 514 and optionally other conduits and the second half 590 may serve primarily as a fluid tight cover to the channels formed in the surface of the first half 588.

The cam assembly 548 includes the cam shaft 482 and may be used as discussed above and shown in the embodiment of FIG. 28. The cam assembly 548 includes the cam shaft 482 with cam lobes which are configured and timed to sequentially translate the inlet pushrod, outlet pushrod and vent pushrod of the respective inlet valve 488, outlet valve 492 and displacement diaphragm 494 of the pump chamber 476 in a sequence that moves therapeutic fluid 50 or any other suitable fluid therethrough. For the medical pump system embodiment 540 shown in FIG. 30, if the primary pump cam shaft 482 is rotated by the rotational actuator (e.g. the gear motor 84) in a standard rotation direction, the pump chamber assembly 407 (that may include the pump inlet valve 488, which may be the same as or similar to the inlet valve assembly 75 discussed above, pump outlet valve 492, which may be the same as or similar to the outlet valve assembly 79 discussed above, displacement diaphragm 494, which may be the same as or similar to the resilient displacement membrane 64 discussed above, and primary pump cam 482) will pump fluid from the selected fluid volume (either 122 or 122ʹ) or filtered air 477 from the inlet conduit 514 and out of the outlet conduit 56. The primary pump cam 482 may actuate the pump chamber assembly 407 to pump fluid from the selected fluid volume (either 122 or 122ʹ) or filtered air 477 from the inlet conduit 514 out of the outlet conduit 56 in a manner the same as or similar to that discussed above and shown in FIGS. 14A, 14B and 14C.

The cam assembly 548 also includes the cam segment 550. The cam segment 550 includes cam lobes which are configured to sequentially and controllably position the pushrods of the respective selector valves 508, 510, 556 associated with the selector valve assembly 552 in a sequence that is configured to controllably select a single source of therapeutic fluid 50 or any other suitable fluid for supplying the pump chamber assembly 407. For the embodiments shown, the reservoir selector assembly 554 includes both the selector valve assembly 552 and an actuator such as the cam segment 550. This selector valve assembly 552 of the reservoir selector assembly 554 may include at least one selector valve for each separate fluid volume 122, 122ʹ as well as one selector valve for the filtered air inlet conduit 514. The two fluid volumes 122, 122ʹ and their respective associated first selector valve 508 and a second selector valve 510 are illustrated as an example in the embodiment of FIG. 30 as well as the third selector valve 556 which is disposed in fluid and operative communication with the filtered air inlet conduit 514. In some cases, such a selector valve assembly 552 including selector valves 508, 510, 556 may be controlled with the cam segment 550 that may be coaxially coupled with the primary pump cam shaft 482 and the rotational actuator of the cam shaft 482 such as the motor 84 through a one way coupling.

During operation of the reservoir selector assembly 554, if the motor 84 and cam shaft 482 are operated in a reversed rotational direction opposite to that of the pumping rotational direction, a selector valve coupling, such as the selector valve coupling 416 in the form of the spring clutch 496 or other type of one way coupling, engages the cam segment 550 and rotates the cam segment 486 in the reverse direction to position the cam lobes of the cam segment 550 and associated valve assemblies 508, 510, 556 of the selector valve assembly 552 so as to close all of the valves associated with non-selected fluid volumes 122, 122ʹ or filtered air inlet conduit 514. With the valves associated with the non-selected fluid sources closed, the cam lobes are also set to open the selector valve of the selected fluid volume 122, 122ʹ or filtered air inlet conduit 514 to make the contents and internal volume of the selected fluid volume or filtered air inlet conduit 514 available for flow through the pump mechanism 407.

In some cases, when the motor 84 is operated in the reversed non-pumping valve selection direction and the cam shaft 482 is operated in this same non-pumping valve selection direction, pumping fluid through the pump chamber 476 in a backward direction away from the patient 142 may be avoided by liming the rotational translation of the cam shaft 482 in this reverse direction. For some embodiments, the controller 88 may be configured to limit rotating translation of the cam shaft 482 in the reverse valve selection direction to about 100 degrees or less. Avoiding pumping of fluid in a reverse direction may also be facilitated by positioning the end point of the reverse rotation to a known rotational position of the cam shaft 482 where it will not cause undesired fluid flow such as the typical stop point of the cam shaft 482 just after the pump outlet valve 492 and vent valve 502 have opened. Reversing the cam shaft 482 from this point by about 100 degrees of rotation or less may typically re-close the pump outlet valve 492 and vent valve 502 and possibly re-open the pump inlet valve 488, but stops before any fluid disposed in the pump chamber 476 is displaced back to the reservoir selector assembly 554. If rotational displacement of the cam segment 550 of more than about 100 degrees to about 120 degrees is required for a desired setting of the reservoir selector assembly 554, this may be carried out in two separate steps of about 100 degrees reverse rotation or less. That is, the cam shaft 482 would be rotated in the standard pumping direction to the typical end point discussed above, then reversed until the cam segment 550 was rotated backwards by about 80 degrees to about 100 degrees thereby resetting the selection of the reservoir selector assembly 554 in a first step. The cam shaft 482 may then be rotated in the forward direction back to the desired end point discussed above whereupon the rotational direction of the cam shaft 482 may yet again be reversed to the non-pumping valve selection direction so as to engage the cam segment 550 and rotate the cam segment in the valve selection direction again by about 80 degrees to about 100 degrees giving a cumulative reverse rotational displacement of the cam segment 550 of about 160 degrees to about 200 degrees. Since the valve selection states of the reservoir selector assembly 554 may be about 90 degrees apart in some instances, this reverse rotational displacement would be sufficient for toggling through two different valve selection settings. This process could be repeated as many times as necessary to achieve the desired valve selection state of the reservoir selector assembly 554 without pumping a large amount of fluid through the pump chamber 476.

For some medical pump system embodiments 540 that include three fluid sources to select from, the cam lobes of the cam segment 550 may be configured to scroll through discrete selection states for each fluid source sequentially in a repeating fashion. For example, some cam segments 550 may be configured to switch from selecting the first fluid volume 122, then the second fluid volume 122ʹ, then the filtered air 477 from the inlet conduit 514 and then back to the first fluid volume 122 and so on. However, for some embodiments, such as the embodiment shown in FIGS. 31-40, it may be more efficient from an operating standpoint to interleave the selection of the filtered air 477 fluid source between each selection of the first fluid volume 122 and the second fluid volume 122ʹ. As such, the sequence for the configuration shown in FIGS. 31-40 may be to select the first fluid volume 122, then the filtered air 477 from the inlet conduit 514, then the second fluid volume 122ʹ, back to the filtered air 477 from the inlet conduit 514 and then back to the first fluid volume 122 again and so on. Such an arrangement might be more efficient if it is known that there will be an air purge between the delivery of each different therapeutic fluid from each of the respective fluid volumes 122, 122ʹ. Of course this sequence may begin at any desired starting state, i.e., it does not have to begin with the first fluid volume 122 selected.

FIGS. 35A-35C illustrate schematically three different fluid source selection states of the reservoir selector assembly embodiment 554 illustrated in FIGS. 30-40. Referring to FIG. 35A, the reservoir selector assembly embodiment 554 is shown with the first fluid volume 122 selected for pumping through the inlet conduit 42 into the pump chamber 476. In FIG. 35A, the third cam lobe 572 of the cam segment 550 is fully extended against the third selector pushrod 570 so as to seal the resilient selector valve membrane 491 against the inlet port 514 of the filtered air 477 of the third selector valve assembly 556 so as to prevent fluid communication between the inlet port 42 of the pump chamber 476 and the inlet port 514. For some embodiments, the selector valve membrane 491 may include dimples 58 which are disposed towards the fluid source ports and positioned to seal against those respective ports when pressed against them. The dimples 58 shown in FIGS. 35A-35C are sufficiently large to seal the respective inlet ports and still allow for a gap to be disposed between the nominal upper surface of the selector valve membrane 491 and the selector valve assembly housing. Such gaps may be useful for allowing the fluids to flow past a closed selector valve assembly.

Referring again to FIG. 35A, the second cam lobe 489 of the cam segment 550 is fully extended against the second selector pushrod 493 so as to seal the respective dimple 58 of the resilient selector valve membrane 491 against the inlet port of the second selector valve assembly 510 so as to prevent fluid communication between the inlet port 42 of the pump chamber 476 and the second fluid volume 122ʹ. In this same cam segment 550 position as shown, the first cam lobe 487 is in a retracted position allowing the resilient selector valve membrane 491 and first selector pushrod 490 to retract away from the inlet port of the first selector valve assembly 508 thereby permitting fluid communication between the first fluid volume 122 and the inlet port 42 of the pump chamber 476. The retraction force applied to the first selector pushrod 490 (and the other selector pushrods 493 and 570) may be supplied by the resilient selector valve membrane 491 which may be configured and positioned to apply a resilient preload on the pushrods 490, 493, 570.

Referring to FIG. 35B, the reservoir selector assembly embodiment 554 is shown with the second fluid volume 122 selected for pumping through the inlet conduit 42 into the pump chamber 476. In FIG. 35B, the third cam lobe 572 of the cam segment 550 is fully extended against the third selector pushrod 570 so as to seal the resilient selector valve membrane 491 against the inlet port 514 of the filtered air 477 of the third selector valve assembly 556 so as to prevent fluid communication between the inlet port 42 of the pump chamber 476 and the inlet port 514. The first cam lobe 487 is fully extended against the first selector pushrod 490 so as to seal the resilient selector valve membrane 491 against the inlet port of the first selector valve assembly 508 so as to prevent fluid communication between the inlet port 42 of the pump chamber 476 and the first fluid volume 122. In this same cam segment 550 position as shown, the second cam lobe 489 is in a retracted position allowing the resilient selector valve membrane 491 and second selector pushrod 493 to retract away from the inlet port of the second selector valve assembly 510 thereby permitting fluid communication between the second fluid volume 122ʹ and the inlet port 42 of the pump chamber 476.

Referring to FIG. 35C, the reservoir selector assembly embodiment 554 is shown with the filtered air 477 fluid source from the inlet conduit 514 selected for pumping through the inlet conduit 42 into the pump chamber 476. In FIG. 35C, the second cam lobe 489 is fully extended against the second selector pushrod 493 so as to seal the resilient selector valve membrane 491 against the inlet port of the second fluid volume 122ʹ of the second selector valve assembly 510 so as to prevent fluid communication between the inlet port 42 of the pump chamber 476 and the second fluid volume 122ʹ. The first cam lobe 487 is fully extended against the first selector pushrod 490 so as to seal the resilient selector valve membrane 491 against the inlet port of the first selector valve assembly 508 so as to prevent fluid communication between the inlet port 42 of the pump chamber 476 and the first fluid volume 122. In this same cam segment 550 position as shown, the third cam lobe 572 is in a retracted position allowing the resilient selector valve membrane 491 and third selector pushrod 570 to retract away from the inlet port of the third selector valve assembly 556 thereby permitting fluid communication between the filtered air 477 from the inlet conduit 514 and the inlet port 42 of the pump chamber 476.

For some embodiments of the cam segment, the cam lobes 487, 489, 572 may provide a total lift when in the extended position of about 0.4 mm to about 1 mm. In addition, for some embodiments, the cam lobes 487, 489, 572 and associated pushrods 490, 493, 570 may have a pre-tensioning preload applied to them by the selector valve membrane 491 of about 0.1 mm to about 0.5 mm when in the retracted position away from the fluid source inlets.

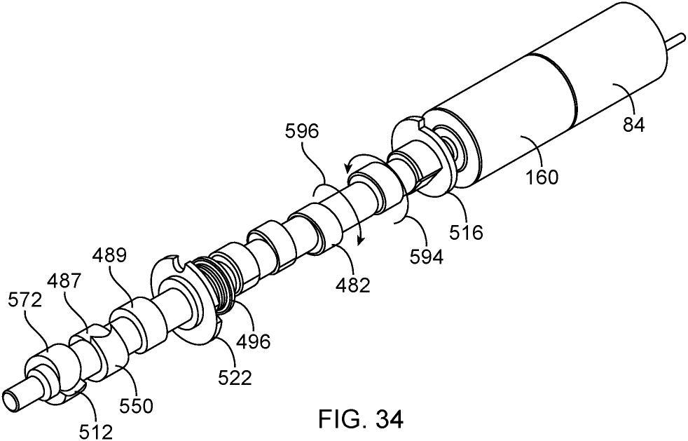

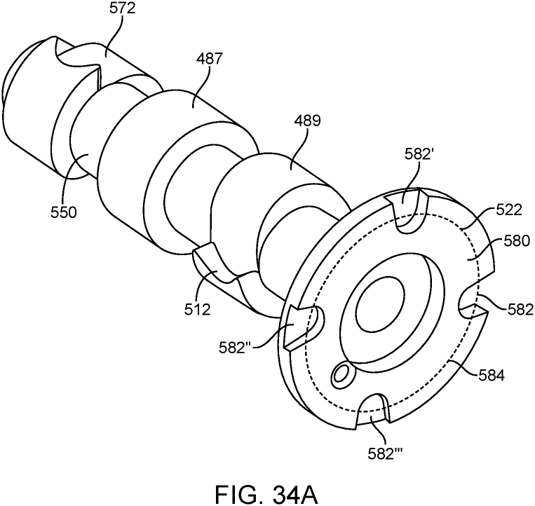

The rotational position of the cam segment 550 relative to the selector valve assembly 552 while disposed within each of the fluid source selection states of FIGS. 35A, 35B and 35C (as well as others) may be detected by the second optical detector 524 which is operatively coupled to the second fiducial flag 522 which is disposed in fixed relation to the cam segment 550. Referring to FIG. 34A, embodiments of the second fiducial flag 522 may include a disc or plate 580 having a continuous nominal transverse thickness with parallel sides. For analog embodiments of the optical detector 524, the plate 580 of the second fiducial flag 522 may be made from a translucent material that has indicator points 582, 582ʹ, 582ʺ, 582ʹʺ disposed about a constant radius ring 584 of the plate 580. The translucent material may include any rigid translucent materials such a polymers including plastics such as Delrin®, ABS plastic, PVC or the like. Each distinct indicator point 582 may consist of a zone of constant thickness that differs from the nominal transverse thickness of the plate 580, including optionally a zone of zero thickness (as is shown for indicator point 582) .

The second optical detector 524 may be configured to transmit optical energy through the constant radius ring 584 of the plate 580 that transects the indicator points as the cam segment 550 rotates and measure the attenuation of the optical energy due to absorption of the optical energy by the varying thicknesses. For such a second fiducial flag 522 that has four different indicator points 582, 582ʹ, 582ʺ, 582ʹʺ distributed at different angular positions, every 90 degrees for example, with each of those positions corresponding to a specific selection state, then the controller 88 which is in operative communication with the optical detector 524, can know which position the cam segment 550 is in and which fluid selection state the reservoir selector assembly 554 is in. A similar arrangement may optionally be used for the first fiducial flag 516 and first optical detector 518 of the cam shaft 482 in some cases.

In order to control the pressure within the rigid outer container 20 and about each of the fluid volumes 122, 122ʹ, the vent conduit 498, which may be the same as or similar to the vent conduit 111 discussed above, may be disposed in fluid communication between a vent valve assembly 502, which may be the same as or similar to the vent valve assembly 109 discussed above, and the respective interior air volume 24 of the rigid outer container 20 as shown in FIG. 30. The respective fluid volumes 122, 122ʹ are disposed within the same rigid outer container 20 and, as such, are at the same pressure. However, any other suitable arrangement for the vent conduit 498 and associated fluid volumes 122, 122ʹ may be used. The vent valve assembly 502 is configured to be actuated by rotation of the cam assembly 548 in order to selectively vent the interior volume 24 of the rigid outer container 20 in order to release negative pressure build up due to dispensing of therapeutic fluids 50 or the like from the fluid volumes 122, 122ʹ or filtered air 477 which is taken from within the interior volume 24 of the rigid outer container 20, passed through the sterility filter 515 and then drawn through the inlet conduit 514 as indicated by arrow 560. In conjunction with predetermined venting protocols, the pressure sensor 130 may be used to make volume determinations, alarm conditions, etc. as discussed above.

Some embodiments of the medical pump system 540 may include the reservoir cartridge assembly 542 having a first fluid source in the form of the first fluid volume 122, a second fluid source in the form of the second fluid volume 122ʹ, and the reservoir selector assembly 554. The reservoir selector assembly 554 includes the first selector valve assembly 508 disposed in fluid communication with the first fluid volume 122 and the second selector valve assembly 510 disposed in fluid communication with the second fluid volume 122ʹ. The reservoir cartridge assembly 542 may also include the pump chamber assembly 407 including the pump chamber 476 having an interior volume, the inlet port 42 in fluid communication with the reservoir selector assembly 554 and an outlet port 56 which is disposed in fluid communication with the interior volume of the pump chamber 476 and with the outlet conduit 56.

The actuator assembly 544 of this medical pump system embodiment 540 may be configured to be operatively and releasably coupled to the reservoir cartridge assembly 542. The actuator assembly 544 may include the cam assembly 548 having the cam shaft 482 which is configured to be operatively coupled to the pump chamber assembly 407, and the cam segment 550 which is configured to be operatively coupled to the reservoir selector assembly 554. The motor 84 of the actuator assembly 544 may be operatively coupled to the cam assembly 548 with the controller 88 also operatively coupled to the motor 84. For some embodiments, when the reservoir cartridge assembly 542 and actuator assembly 544 are operatively coupled together, the pump chamber assembly 407 may be actuated by the cam assembly 548 and fluid may be pumped through the pump chamber assembly in the same manner as is discussed above and illustrated in FIGS. 14A, 14B and 14C. Venting of the air volume 24 may also be carried out similarly to the methods discussed above with regard to the same embodiments.

In some cases, the reservoir selector assembly 554 may further include the third selector valve assembly 556 which is disposed in fluid communication with a third fluid source which may include a source of filtered air such as the inlet conduit 514 and sterility filter 515. Also, for some embodiments, the third fluid source may include a third fluid volume (not shown) which may have the same features, dimensions and materials as those of the first fluid volume 122 and second fluid volume 122ʹ. The first fluid source 122 and the second fluid source 122ʹ are disposed within the interior volume of the rigid outer container 20. In some instances, the interior volume of the outer rigid container 20 may include the air volume 24. In such cases, the first fluid volume 122 may be separated from the air volume 24 by the first flexible membrane 28 disposed about the first fluid volume 122 and the second fluid volume 122ʹ may be separated from the air volume 24 by the second flexible membrane 28ʹ disposed about the second fluid volume. For some embodiments, the reservoir selector assembly may be actuated and function to select a particular fluid source to the exclusion of all others in the medical pump system embodiment in a manner which is the same as or similar to that shown in FIGS. 35A, 35B and 35C and discussed above.

For some embodiments, the pump chamber assembly may further include the vent valve assembly 502 which is operatively coupled in fluid communication to the air volume 24 of the interior volume rigid outer container 20. For such embodiments, the third selector valve assembly 556 may be disposed in fluid communication with the inlet conduit 514 in fluid communication with the sterility filter 515 with the sterility filter 515 being disposed within the air volume 24 of the rigid outer container 20. In such cases, filtered air that is pumped through the pump chamber 476 may first enter the sealed enclosure (formed by the reservoir cartridge assembly and actuator assembly when they are operatively coupled together) through the air filter 562 which may include a hydrophobic air filter. The air within the sealed enclosure may be essentially vented through the hydrophobic filter 562 which may be disposed and sealed over an aperture of the sealed enclosure. The aperture is typically in fluid communication with the environment disposed about the medical pump system 540. Once the air enters the sealed enclosure between the reservoir cartridge assembly 542 and the actuator assembly 544, the air may then pass through the vent valve assembly 502 (when the vent valve assembly is in an open state) and into the air volume 24 of the rigid outer container 20. Once in the air volume 24 of the rigid outer container 20, the air may then pass through the filter 515 (which may optionally include the sterility filter) and into the inlet conduit 514 and ultimately into the third selector valve assembly 556 and inlet port 42 of the pump chamber 476.

Some embodiments of this medical pump system may also include the pressure sensor 130 which is operatively coupled to the interior volume of the rigid outer container 20 and configured to detect a pressure within the interior volume 24 of the rigid outer container 20 and also within the first fluid volume 122 and the second fluid volume 122ʹ which are all the same. In some cases, the pressure sensor 130 is disposed on the actuator cartridge 544.

Regarding the cam assembly 548, the selector valve coupling 416 operatively couples the cam segment 550 to the cam shaft 482. As discussed above, in some instances, the selector valve coupling 416 includes the spring clutch which is configured to slip when the cam shaft 482 is rotated in the pumping direction, as indicated by arrow 594 in FIG. 34, and engage the cam segment 550 when the cam shaft 482 is rotated in the opposite non-pumping valve selection direction, as indicated by arrow 596 in FIG. 34. The cam segment 55 may also include the first cam lobe 487 and the second cam lobe 489, each of which includes the ratcheting feature 512 which is configured to stop rotation of the cam segment 550 in the valve selecting non-pumping direction 596. In some cases, the ratcheting feature 512 of each of the first cam lobe 487 and second cam lobe 489 includes a step structure 512 on the respective cam lobes 487, 489 which is arranged to contact and abut portions of a respective selector valve assembly 508, 510 of the reservoir selector assembly 554 as shown in FIGS. 34 and 34A.

For some embodiments, the cam shaft 482 of the cam assembly 548 includes the first fiducial flag 516 which is operatively coupled to the first optical reader 518. The first optical reader 518 is in operative communication with the controller 88. The first optical reader 518 is configured to detect the rotational position of the first fiducial flag 516 and a corresponding rotational position of the cam shaft 482 as shown in FIGS. 35A-35C. In addition, the cam segment 550 of the cam assembly 548 may include the second fiducial flag 522 which is operatively coupled to the second optical reader 524 which is in operative communication with the controller 88. The second optical reader 524 is configured to detect the rotational position of the second fiducial flag 522 and the corresponding rotational position of the cam segment 550. The rotational position of the cam segment 550 may be used to determine the open/closed state of the first selector valve assembly 508, second selector valve assembly 510 and third selector valve assembly 556 and thus determine which fluid source of the first, second and third fluid sources is being made available for pumping through the pump chamber 476 and into the outlet conduit 56 to the patient 142.

In use, some embodiments of a method of delivering multiple fluids to a patient from the medical pump system 540, or any other suitable medical pump system embodiment discussed herein, may include actuating the cam shaft 482 of the cam assembly 548 by rotation of the cam shaft 482 in a pumping direction 594 and pumping a first fluid from the first fluid source which may include the first fluid volume 122. The first fluid is pumped through the first selector valve assembly 508 of the reservoir selector assembly 554 and the pump chamber assembly 407 of the medical pump system 540 into the outlet conduit 56 and to the patient 142 while the second selector valve assembly 510 and third selector valve assembly 556 of the reservoir selector assembly 554 are closed.

Thereafter, the method may include reversing the rotation of the cam shaft 482 of the cam assembly 548 from the pumping direction 594 to the valve selection direction 596, ceasing pumping of the first fluid from the first fluid volume 122 and rotating the cam segment 550 of the cam assembly 548 in the valve selection direction 596 to close the first selector valve assembly 508, close the third selector valve assembly 556 and open the second selector valve assembly 510. The open/closed state of the selector valve assemblies may be detected and/or confirmed by the rotational position of the second fiducial flag 522 and corresponding rotational position of the cam segment 550. The method embodiment may also include subsequently reversing the rotation of the cam shaft 482 from the valve selection direction 596 to the pumping direction 594 and pumping the second fluid from the second fluid volume 122ʹ through the second selector valve assembly 510 and pump chamber assembly 407 of the medical pump system 540 into the outlet conduit 56 and to the patient 142 while the first selector valve assembly 508 and the third selector valve assembly 556 are closed.

After pumping the second fluid from the second fluid volume 122ʹ, the rotation of the cam shaft 482 of the cam assembly 548 may again be reversed from the pumping direction 594 to the valve selection direction 596. Pumping of the second fluid from the second fluid volume 122ʹ is ceased and the cam segment 550 of the cam assembly 548 rotated in the valve selection direction 596 so as to close the first selector valve assembly 508, close the second selector valve assembly 510 and open the third selector valve assembly 556. Finally, the rotation of the cam shaft 482 may again be subsequently reversed from the valve selection direction 596 to the pumping direction 594. A third fluid, such as filtered air 477, is then pumped from the third fluid source which may include the inlet conduit 514 coupled to the filter 515. The pumped third fluid may be carried out through the third selector valve assembly 556 and pump chamber assembly 407 of the medical pump system 540 into the outlet conduit 56 and to the patient 142 while the first selector valve assembly 508 and second selector valve assembly 510 are closed. In addition, for some embodiments, the selector valve assemblies 508, 510, 556 may be actuated and function to select a particular fluid source to the exclusion of all others in the medical pump system embodiment in a manner which is the same as or similar to that shown in FIGS. 35A, 35B and 35C and discussed above.

For some such method embodiments, pumping the first fluid may include pumping a first therapeutic liquid 50 through the pump chamber 476 and outlet conduit 56, pumping the second fluid may include pumping a bolus of filtered air 477 through the pump chamber 476 and outlet conduit 56 after pumping the first therapeutic liquid 50. Pumping the third fluid may include pumping a second therapeutic liquid 50ʹ through the pump chamber 476 and outlet conduit 56 after pumping the bolus of filtered air 477 therethrough. In some cases, pumping the bolus of filtered air 477 may include forming a meniscus 480, as shown in FIGS. 29A and 29B, at the boundary between the bolus of filtered air 477 and the first therapeutic liquid 50 and forming a meniscus 480 at the boundary between the bolus of filtered air 477 and the second therapeutic liquid 50ʹ. The pumping direction is indicated by arrows 479 in FIGS. 29A and 29B. Such method embodiments may include substantially purging the first therapeutic liquid 50 from the pump chamber 476 and outlet conduit 56 prior to passage of the second therapeutic liquid 50ʹ through the pump chamber 476 and outlet conduit 56 as well as the port 145 and associated hollow cannula 147. For some such embodiments, pumping the bolus of filtered air 477 through the pump chamber 476 and outlet conduit 56 may include pumping a bolus of sterilized air 477 through the pump chamber 476 and outlet conduit 56. The bolus of sterilized air may be formed by passing the air through the filter 515 which may include a sterilizing filter having a pore size of about 0.22 µm or less.

In some instances, the method embodiment may also include detecting the pressure within the interior volume 24 of the rigid outer container 20 during the pumping and selector valve selection processes. The rigid outer container 20 is disposed and sealed about the first fluid volume 122, second fluid volume 122ʹ and third fluid source which may include the inlet conduit 514 and filter 515. The pressure sensor 130 may be operatively coupled in fluid communication with the interior volume of the rigid outer container 20 and operatively coupled to the controller 88 of the medical pump system 540. As discussed above, the rotational position of the cam shaft 482 may be detected during the method with the first fiducial flag 516 which is secured to the cam shaft 482 and which is operatively coupled to the first optical reader 518 which is in operative communication with the controller 88. The rotational position of the cam segment 550 may also be detected and determined with the second fiducial flag 522 which is secured to the cam segment 550 and which is operatively coupled to the second optical reader 524 which is disposed in operative communication with the controller 88.

Embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation and use of such terms and expressions do not exclude any equivalents of the features shown and described or portions thereof, and various modifications are possible. The term “a” or “an” can refer to one of or a plurality of the elements it modifies (e.g., “a reagent” can mean one or more reagents) unless it is contextually clear either one of the elements or more than one of the elements is described. Thus, it should be understood that although embodiments have been specifically disclosed by representative embodiments and optional features, modification and variation of the concepts herein disclosed may be resorted to by those skilled in the art, and such modifications and variations are considered within the scope of this disclosure.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A medical pump system, comprising:
a reservoir cartridge assembly, comprising:
a first fluid source,
a second fluid source,
a reservoir selector assembly including a first selector valve assembly disposed in fluid communication with the first fluid source and second selector valve assembly disposed in fluid communication with the second fluid source,
a pump chamber assembly including:
a pump chamber having an interior volume,
an inlet port in fluid communication with the reservoir selector assembly, and
an outlet port in fluid communication with the interior volume of the pump chamber and with an outlet conduit; and
an actuator assembly that is configured to be operatively and releasably coupled to the reservoir cartridge assembly, comprising:
a pump chamber actuator,
a motor operatively coupled to the pump chamber actuator, and
a controller operatively coupled to the motor.

2. The medical pump system of claim 1 wherein the pump chamber actuator comprises a cam assembly including a cam shaft which is configured to be operatively coupled to the pump chamber assembly, and a cam segment which is configured to be operatively coupled to the reservoir selector assembly.

3. The medical pump system of claim 1 wherein the reservoir cartridge assembly further comprises a third fluid source and the reservoir selector assembly further comprises a third selector valve assembly disposed in fluid communication with the third fluid source.

4. The medical pump system of claim 3 wherein the third fluid source comprises a third fluid volume.

5. The medical pump system of claim 3 wherein the third fluid source comprises a source of filtered air.

6. The medical pump system of claim 5 wherein the source of filtered air comprises an inlet conduit in fluid communication with a sterility filter.

7. The medical pump system of claim 5 wherein the first fluid source and the second fluid source are disposed within an interior volume of a rigid outer container.

8. The medical pump system of claim 7 wherein the interior volume of the outer rigid container includes an air volume and the first fluid source includes a first fluid volume which is separated from the air volume by a first flexible membrane disposed about the first fluid volume and the second fluid source includes a second fluid volume which is separated from the air volume by a second flexible membrane disposed about the second fluid volume.

9. The medical pump system of claim 8 wherein the pump chamber assembly further comprises a vent valve assembly which is operatively coupled in fluid communication to the air volume of the interior volume of the rigid outer container.

10. The medical pump system of claim 9 wherein the third selector valve assembly is disposed in fluid communication with the inlet conduit of the third fluid source, the inlet conduit being in fluid communication with a sterility filter which is disposed within the air volume of the rigid outer container.

11. The medical pump system of claim 1 wherein the reservoir cartridge assembly and actuator assembly form a sealed enclosure when operatively coupled together.

12. The medical pump system of claim 11 wherein the enclosure of operatively coupled reservoir cartridge assembly and actuator assembly is vented through a hydrophobic filter disposed and sealed over an aperture of the enclosure that is in communication with the environment disposed about the medical pump system.

13. The medical pump system of claim 7 further comprising a pressure sensor which is operatively coupled to an interior volume of the rigid outer container and configured to detect a pressure within the interior volume of the rigid outer container and within the first fluid volume and the second fluid volume.

14. The medical pump system of claim 13 wherein the pressure sensor is disposed on the actuator assembly.

15. The medical pump system of claim 2 further comprising a selector valve coupling that operatively couples the cam segment to the cam shaft.

16. The medical pump system of claim 15 wherein the selector valve coupling comprises a one way coupling which is configured to slip when the cam shaft is rotated in a pumping direction and engage the cam segment when the cam shaft is rotated in an opposite valve selection direction.

17. The medical pump system of claim 16 wherein the one way coupling comprises a spring clutch.

18. The medical pump system of claim 16 wherein the cam segment further comprises a first cam lobe and a second cam lobe, each of which comprises a ratcheting feature configured to stop rotation of the cam segment in the opposite non-pumping direction.

19. The medical pump system of claim 18 wherein the ratcheting feature of each of the first cam lobe and second cam lobe comprises a step structure on the respective cam lobes which is arranged to contact and abut a respective selector valve assembly of the reservoir selector assembly.

20. The medical pump system of claim 2 wherein the cam shaft of the cam assembly comprises a first fiducial flag which is operatively coupled to a first optical reader which is in operative communication with the controller, the first optical reader configured to detect the rotational position of the first fiducial flag and a corresponding rotational position of the cam shaft.

21. The medical pump system of claim 20 wherein the cam segment of the cam assembly comprises a second fiducial flag which is operatively coupled to a second optical reader which is in operative communication with the controller, the second optical reader configured to detect the rotational position of the second fiducial flag and corresponding rotational position of the cam segment.

22. A method of delivering multiple fluids to a patient from a medical pump system, comprising:
actuating a pump chamber assembly with a cam shaft of a cam assembly by rotating the cam shaft in a pumping direction and pumping a first fluid from a first fluid source through a first valve assembly of a reservoir selector assembly and through a pump chamber assembly of the medical pump system into an outlet conduit and to the patient,
reversing the rotation of the cam shaft from the pumping direction to a valve selection direction and ceasing pumping of the first fluid from the first fluid source by the pump chamber assembly,
rotating a cam segment of the cam assembly in the valve selection direction with a one way coupling disposed between and operatively coupling the cam shaft to the cam segment to close the first valve assembly and open a second valve assembly of the reservoir selector assembly, and
reversing the rotation of the cam shaft from the valve selection direction to the pumping direction and pumping a second fluid from the second fluid source through the second valve assembly and pump chamber assembly of the medical pump system into the outlet conduit and to the patient.

23. A method of delivering multiple fluids to a patient from a medical pump system, comprising:
actuating a pump chamber assembly with a cam shaft of a cam assembly by rotating the cam shaft in a pumping direction and pumping a first fluid from a first fluid source through a first valve assembly of a reservoir selector assembly and through a pump chamber assembly of the medical pump system into an outlet conduit and to the patient while a second valve assembly and third valve assembly of the reservoir selector assembly are closed,
reversing the rotation of the cam shaft of the cam assembly from the pumping direction to a valve selection direction, ceasing pumping of the first fluid from the first fluid source and actuating the reservoir selector assembly by rotating a cam segment of the cam assembly in a valve selection direction with a one way coupling disposed between and operatively coupling the cam shaft to the cam segment to close the first valve assembly, close the third valve assembly and open the second valve assembly,
reversing the rotation of the cam shaft from the valve selection direction to the pumping direction and pumping a second fluid from a second fluid source through the second valve assembly and pump chamber assembly of the medical pump system into the outlet conduit and to the patient while the first valve assembly and the third valve assembly are closed,
reversing the rotation of the cam shaft from the pumping direction to the valve selection direction and ceasing pumping of the second fluid from the second fluid source and rotating the cam segment of the cam assembly in the valve selection direction and closing the first valve assembly, closing the second valve assembly and opening the third valve assembly, and
reversing the rotation of the cam shaft from the valve selection direction to the pumping direction and pumping a third fluid from a third fluid source through the third valve assembly and pump chamber assembly of the medical pump system into the outlet conduit and to the patient while the first valve assembly and second valve assembly are closed.

24. The method of claim 23 wherein pumping the first fluid comprises pumping a first therapeutic liquid through the pump chamber and outlet conduit, pumping the second fluid comprises pumping a bolus of filtered air through the pump chamber and outlet conduit, and pumping the third fluid comprises pumping a second therapeutic liquid through the pump chamber and outlet conduit.

25. The method of claim 24 further comprising forming a meniscus at a boundary between the bolus of filtered air and the first therapeutic liquid and forming a meniscus at a boundary between the bolus of filtered air and the second therapeutic liquid.

26. The method of claim 25 further comprising substantially purging the first therapeutic liquid from the pump chamber and outlet conduit by passage of the menisci and surface tension of the first therapeutic fluid prior to passage of the second therapeutic liquid through the pump chamber and outlet conduit.

27. The method of claim 24 wherein pumping a bolus of filtered air through the pump chamber and outlet conduit comprises pumping a bolus of sterilized air through the pump chamber and outlet conduit.

28. The method of claim 23 further comprising detecting a pressure within an interior volume of a rigid outer container that is disposed and sealed about the first fluid source, second fluid source and third fluid source with a pressure sensor that is operatively coupled in fluid communication with the interior volume of the rigid outer container and operatively coupled to a controller of the medical pump system.

29. The method of claim 23 further comprising detecting the rotational position of the cam shaft with a first fiducial flag which is secured to the cam shaft and which is operatively coupled to a first optical reader.

30. The method of claim 23 further comprising detecting the rotational position of the cam segment with a second fiducial flag which is secured to the cam segment and which is operatively coupled to a second optical reader.

31. A method of delivering multiple fluids to a patient from a medical pump system, comprising:
pumping a first liquid from a first fluid source of the medical pump system into an outlet conduit and to the patient,
ceasing pumping of the first liquid,
pumping a gas from a second fluid source into the outlet conduit and to the patient,
ceasing pumping of the gas from the second fluid source, and
pumping a third liquid from a third fluid source into the outlet conduit and to the patient.

32. The method of claim 31 wherein pumping the first liquid comprises pumping a first therapeutic liquid into the outlet conduit and to the patient, pumping the gas comprises pumping a bolus of filtered air into the outlet conduit and to the patient, and pumping the third liquid comprises pumping a second therapeutic liquid into the outlet conduit and to the patient.

33. The method of claim 32 further comprising forming a meniscus at a boundary between the bolus of filtered air and the first therapeutic liquid and forming a meniscus at a boundary between the bolus of filtered air and the second therapeutic liquid.

34. The method of claim 33 further comprising substantially purging the first therapeutic liquid from the outlet conduit by passage of the menisci and acting surface tension of the first therapeutic liquid prior to passage of the second therapeutic liquid through the outlet conduit.

35. The method of claim 32 wherein pumping a bolus of filtered air into the outlet conduit comprises pumping a bolus of sterilized air through into the outlet conduit and to the patient.

36. A medical pump system, comprising:
a reservoir cartridge assembly, comprising:
a first fluid reservoir,
a second fluid reservoir,
a reservoir selector assembly in fluid communication with the first fluid reservoir and the second fluid reservoir,
a pump chamber assembly including:
a pump chamber having an interior volume which is at least partially bounded by a pump housing,
an inlet port in fluid communication with the reservoir selector assembly,
a resilient inlet membrane which is disposed adjacent the inlet port, which is spaced from the inlet port when in a relaxed state, and which is sufficiently distendable towards the inlet port to seal the inlet port in a compressed state,
an outlet port in fluid communication with the interior volume and with an outlet conduit,
a resilient outlet membrane which is disposed adjacent the outlet port, which is spaced from the outlet port when in a relaxed state, and which is sufficiently distendable towards the outlet port to seal the outlet port in a compressed state,
a displacement chamber disposed within the interior volume,
a resilient displacement membrane which is disposed adjacent the displacement chamber, which forms at least a portion of a boundary of the displacement chamber, which is sufficiently inwardly distendable from a relaxed state to reduce the volume of the displacement chamber when in a compressed state, and which is sufficiently resilient to rebound and increase the volume of the displacement chamber when released from the compressed state,
an actuator assembly that is configured to be operatively and releasably coupled to the reservoir cartridge assembly, comprising:
a cam assembly including
a cam shaft comprising
an inlet cam lobe which is operatively coupled to the resilient inlet membrane,
an outlet cam lobe which is operatively coupled to the resilient outlet membrane, and
a displacement cam lobe which is operatively coupled to the displacement membrane, and
a cam segment which is operatively coupled to the reservoir selector assembly,
a motor operatively coupled to the cam assembly, and
a controller operatively coupled to the motor.

37. The medical pump system of claim 36 further comprising a selector valve coupling that operatively couples the cam segment to the cam shaft.

38. The medical pump system of claim 37 wherein the selector valve coupling comprises a spring clutch which is configured to slip when the cam shaft is rotated in a pumping direction and engage the cam segment when the cam shaft is rotated in an opposite non-pumping direction.

39. The medical pump system of claim 38 wherein the cam segment further comprises a first cam lobe and a second cam lobe, each of which comprises a ratcheting feature configured to limit rotation of the cam segment in the opposite non-pumping direction.

40. The medical pump system of claim 39 wherein the ratcheting feature of each of the first cam lobe and second cam lobe comprises a shelf on the respective cam lobes.

41. The medical pump system of claim 36 further comprising a first pressure sensor which is operatively coupled to first fluid reservoir and configured to detect a pressure within the first fluid reservoir and a second pressure sensor which is operatively coupled to the second fluid reservoir and configured to detect a pressure within the second fluid reservoir.

42. The medical pump system of claim 41 wherein the first pressure sensor and the second pressure sensor are disposed on the actuator cartridge.

43. A reservoir cartridge assembly which is configured to be operatively and releasably coupled to an actuator assembly of a medical pump system, comprising:
a reservoir base;
a first fluid reservoir disposed on the reservoir base,
a second fluid reservoir disposed on the reservoir base,
a reservoir selector assembly including a selector valve assembly,
a pump chamber assembly secured in fixed relation to the reservoir base including:
a pump chamber having an interior volume which is at least partially bounded by a pump housing,
an inlet port in fluid communication with the selector valve assembly of the reservoir selector assembly,
a resilient inlet membrane which is disposed adjacent the inlet port, which is spaced from the inlet port when in a relaxed state, and which is sufficiently distendable towards the inlet port to seal the inlet port in a compressed state,
an outlet port in fluid communication with the interior volume and with an outlet conduit,
a resilient outlet membrane which is disposed adjacent the outlet port, which is spaced from the outlet port when in a relaxed state, and which is sufficiently distendable towards the outlet port to seal the outlet port in a compressed state,
a displacement chamber disposed within the interior volume,
a resilient displacement membrane which is disposed adjacent the displacement chamber, which forms at least a portion of a boundary of the displacement chamber, which is sufficiently inwardly distendable from a relaxed state to reduce the volume of the displacement chamber when in a compressed state, and which is sufficiently resilient to increase the volume of the displacement chamber when released from the compressed state.

44. The reservoir cartridge assembly of claim 43 further comprising a vent valve assembly operatively coupled in fluid communication with the first fluid reservoir and the second fluid reservoir.

45. The reservoir cartridge assembly of claim 43 further comprising a power source.

46. The reservoir cartridge assembly of claim 45 wherein the power source comprises a battery.

47. An actuator assembly which is configured to be operatively and releasably coupled to a reservoir cartridge assembly of a medical pump system, comprising:
an actuator chassis;
a controller disposed on the actuator chassis;
a cam assembly which is disposed on the actuator chassis and which includes an inlet cam lobe which is configured to be operatively coupled to a resilient inlet membrane, an outlet cam lobe which is configured to be operatively coupled to a resilient outlet membrane, a displacement cam lobe which is configured to be operatively coupled to a displacement membrane, a vent cam lobe which is configured to be operatively coupled to a vent membrane and a cam segment which is configured to be operatively coupled to a selector valve assembly of a reservoir selector assembly, and
a motor which is operatively coupled to the cam assembly and the controller.

48. The actuator assembly of claim 47 further comprising a pressure sensor which is operatively coupled to the controller and configured to be operatively coupled in fluid communication with a fluid reservoir of a reservoir cartridge assembly.

49. A medical pump system, comprising:
a reservoir cartridge assembly, comprising:
a first fluid reservoir,
a second fluid reservoir,
a source of filtered air,
a reservoir selector assembly in selective fluid communication with the first fluid reservoir, the second fluid reservoir and the source of filtered air, and
a pump chamber assembly in fluid communication with the reservoir selector assembly; and
an actuator assembly that is configured to be operatively coupled to the reservoir cartridge assembly, comprising:
a cam assembly which is operatively coupled to the pump chamber assembly and a cam segment which is operatively coupled to the reservoir selector assembly,
a motor operatively coupled to the cam assembly, and
a controller operatively coupled to the motor.

50. A reservoir cartridge assembly which is configured to be operatively coupled to an actuator assembly of a medical pump system, comprising:
a first fluid reservoir;
a second fluid reservoir;
a source of filtered air;
a reservoir selector assembly in selective fluid communication with the first fluid reservoir, the second fluid reservoir and source of filtered air, the reservoir selector assembly further including a selector valve assembly; and
a pump chamber assembly in fluid communication with the reservoir selector assembly.
